(19) United States
(12) Patent Application Publication
(10) Pub. No.: US 2009/0281054 A1
(43) Pub. Date: Nov. 12, 2009

(54) COMPOSITIONS AND METHODS COMPRISING CAPURAMYCIN ANALOGUES

(76) Inventors: Venkata Reddy, Montgomery Village, MD (US); Marina Nikolaevna Protopopova, Silver Spring, MD (US); Elena Bogatcheva, Bethesda, MD (US)

Correspondence Address: KING & SPALDING, 1180 PEACHTREE STREET , NE, ATLANTA, GA, 30309-3521, US

(21) Appl. No.: 12/331,929

(22) Filed: Dec. 10, 2008

Related U.S. Application Data
(60) Provisional application No. 61/050,889, filed on May 6, 2008.
(60) Provisional application No. 61/107,899, filed on Oct. 23, 2008.

(51) Int. Cl. A61K 31/7068; C07H 19/04; A61P 31/06

(52) U.S. Cl. 514/50; 536/28.53

(57) ABSTRACT

Methods and compositions for treating disease caused by infectious agents, particularly tuberculosis are provided. In particular, methods and compositions comprising substituted derivatives of capuramycin analogs for the treatment of infectious diseases are provided. Also provided are capuramycin analogue formulations comprising PEGylated compounds, including a PEGylated vitamin E derivative, liposomes and nanoparticle carriers. The invention provides methods and compositions comprising a capuramycin analogue and capuramycin analogues in combination with one or more other active agents.

### CROSS REFERENCE TO RELATED APPLICATIONS

This application claims the benefit of co-pending U.S. Provisional Application No. 61/050,889, filed May 6, 2008, and U.S. Provisional Application No. 61/107,899, filed Oct. 23, 2008, both incorporated herein by reference in their entirety.

### FIELD OF INVENTION

The present invention relates to methods and compositions for treating infectious disease and disease caused by microorganisms, including infections caused by mycobacterial agents, such as tuberculosis. In particular, the invention relates to methods and compositions comprising capuramycin and capuramycin analogues of Formula I, Ia, lb, I-A, II, IIa or IIb, formulations of capuramycin or capuramycin analogues and methods and compositions comprising the capuramycin analogues in combination with another active agent.

### BACKGROUND OF THE INVENTION

Mycobacterial infections often manifest as diseases such as tuberculosis. Human infections caused by mycobacteria have been widespread since ancient times, and tuberculosis remains a leading cause of death today. Although the incidence of the disease declined in parallel with advancing standards of living since the mid-nineteenth century, mycobacterial diseases still constitute a leading cause of morbidity and mortality in countries with limited medical resources. Additionally, mycobacterial diseases can cause overwhelming, disseminated disease in immunocompromised patients. In spite of the efforts of numerous health organizations worldwide, the eradication of mycobacterial diseases has never been achieved, nor is eradication imminent. Nearly one third of the world's population is infected with mycobacterium tuberculosis complex, commonly referred to as tuberculosis (TB), with approximately 8 million new cases, and two to three million deaths attributable to TB yearly. Tuberculosis (TB) is the cause of the largest number of human deaths attributable to a single etiologic agent (see Dye et al., J. Am. Med. Association, 282, 677-686, (1999); and 2000 WHO/OMS Press Release).

After decades of decline, TB is now on the rise. In the United States, up to 10 million individuals are believed to be infected. Almost 28,000 new cases were reported in 1990, constituting a 9.4 percent increase over 1989. A sixteen percent increase in TB cases was observed from 1985 to 1990. In 2004 mortality and morbidity statistics included 14.6 million chronic active cases, 8.9 million new cases and 1.6 million deaths, mostly in developing countries. Overcrowded living conditions and shared air spaces are especially conducive to the spread of TB, contributing to the increase in instances that have been observed among prison inmates, and among the homeless in larger U.S. cities. Approximately half of all patients with “Acquired Immune Deficiency Syndrome” (AIDS) will acquire a mycobacterial infection, with TB being an especially devastating complication. AIDS patients are at higher risks of developing clinical TB, and anti-TB treatment seems to be less effective than in non-AIDS patients. Consequently, the infection often progresses to a fatal disseminated disease.

Mycobacteria other than M. tuberculosis are increasingly found in opportunistic infections that plague the AIDS patient. Organisms from the M. avium-intracellulare complex (MAC), especially serotypes four and eight, account for 68% of the mycobacterial isolates from AIDS patients. Enormous numbers of MAC are found (up to 10¹⁰ acid-fast bacilli per gram of tissue), and consequently, the prognosis for the infected AIDS patient is poor.

The World Health Organization (WHO) continues to encourage the battle against TB, recommending prevention initiatives such as the “Expanded Program on Immunization” (EPI), and therapeutic compliance initiatives such as “Directly Observed Treatment Short-Course” (DOTS). For the eradication of TB, diagnosis, treatment, and prevention are equally important. Rapid detection of active TB patients will lead to early treatment by which about 90% cure is expected. Therefore, early diagnosis is critical for the battle against TB. In addition, therapeutic compliance will ensure not only elimination of infection, but also reduction in the emergence of drug-resistance strains.

The emergence of drug-resistant M. tuberculosis is an extremely disturbing phenomenon. The rate of new TB cases proven resistant to at least one standard drug increased from 10 percent in the early 1980's to 23 percent in 1991. Compliance with therapeutic regimens, therefore, is also a crucial component in efforts to eliminate TB and prevent the emergence of drug resistant strains. Equally important is the development of new therapeutic agents that are effective as vaccines, and as treatments, for disease caused by drug resistant strains of mycobacteria.

Multidrug-resistant tuberculosis (MDR TB) is a form of tuberculosis that is resistant to two or more of the primary drugs used for the treatment of tuberculosis. Resistance to one or several forms of treatment occurs when bacteria develop the ability to withstand antibiotic attack and relay that ability to their progeny. Since an entire strain of bacteria inherit this capacity to resist the effects of various treatments, resistance can spread from one person to another.

The World Health Organization estimates that up to 50 million persons worldwide may be infected with drug resistant strains of tuberculosis. Also, 300,000 new cases of MDR-TB are diagnosed around the world each year and 79 percent of the MDR-TB cases now show resistance to three or more drugs routinely used to treat tuberculosis.

In 2003, the Centre for Disease control (CDC) reported that 7.7 percent of tuberculosis cases in the U.S. were resistant to isoniazid, a first line drug used to treat Tuberculosis. The CDC also reported that 1.3 percent of tuberculosis cases in the U.S. were resistant to both isoniazid and rifampin. Rifampin is the drug most commonly used with isoniazid.

Clearly, the possibility of drug resistant strains of tuberculosis that develop during or before treatment are a major concern to health organizations and heath care practitioners. Drugs used in the treatment of tuberculosis include, but are not limited to, Ethambutol, Pyrazinamide, Streptomycin, Isoniazid, Moxifloxacin and Rifampin. The exact course and duration of treatment can be tailored to a specific individual, however several strategies are well known to those skilled in the art.

Although over 37 species of mycobacteria have been identified, more than 95% of all human infections are caused by six species of mycobacteria: M. tuberculosis, M. avium intracellulare, M. kansasii, M. fortuitum, M. chelonae, and M. leprae. The most prevalent mycobacterial disease in humans is tuberculosis (TB) which is predominantly caused by mycobacterial species comprising M. tuberculosis, M. bovis, or M. africanum (Merck Manual 1992). Infection is typically initiated by the inhalation of infectious particles which are able to reach the terminal pathways in lungs. Following engulfment by alveolar macrophages, the bacilli are able to replicate freely, with eventual destruction of the phagocytic cells. A cascade effect ensues wherein destruction of the phagocytic cells causes additional macrophages and lymphocytes to migrate to the site of infection, where they too are ultimately eliminated. The disease is further disseminated during the initial stages by the infected macrophages which travel to local lymph nodes, as well as into the blood stream and other tissues such as the bone marrow, spleen, kidneys, bone and central nervous system. (See Murray et al. Medical Microbiology, The C.V. Mosby Company 219-230 (1990)).

There is still no clear understanding of the factors which contribute to the virulence of mycobacteria. Many investigators have implicated lipids of the cell wall and bacterial surface as contributors to colony morphology and virulence. Evidence suggests that C-mycosides, on the surface of certain mycobacterial cells, are important in facilitating survival of the organism within macrophages. Trehalose 6,6′ dimycolate, a cord factor, has been implicated for other mycobacteria.

The interrelationship of colony morphology and virulence is particularly pronounced in M. avium. M. avium bacilli occur in several distinct colony forms. Bacilli which grow as transparent, or rough, colonies on conventional laboratory media are multiplicable within macrophages in tissue culture, are virulent when injected into susceptible mice, and are resistant to antibiotics. Rough or transparent bacilli, which are maintained on laboratory culture media, often spontaneously assume an opaque R colony morphology, at which time they are not multiplicable in macrophages, are avirulent in mice, and are highly susceptible to antibiotics. The differences in colony morphology between the transparent, rough and opaque strains of M. avium are almost certainly due to the presence of a glycolipid coating on the surface of transparent and rough organisms which acts as a protective capsule. This capsule, or coating, is composed primarily of C-mycosides which apparently shield the virulent M. avium organisms from lysosomal enzymes and antibiotics. By contrast, the non-virulent opaque forms of M. avium have very little C-mycoside on their surface. Both the resistance to antibiotics and the resistance to killing by macrophages have been attributed to the glycolipid barrier on the surface of M. avium.

Diagnosis of mycobacterial infection is confirmed by the isolation and identification of the pathogen, although conventional diagnosis is based on sputum smears, chest X-ray examination (CXR), and clinical symptoms. Isolation of mycobacteria on a medium takes as long as four to eight weeks. Species identification takes a further two weeks. There are several other techniques for detecting mycobacteria such as the polymerase chain reaction (PCR), mycobacterium tuberculosis direct test, or amplified mycobacterium tuberculosis direct test (MTD), and detection assays that utilize radioactive labels.

One diagnostic test that is widely used for detecting infections caused by M. tuberculosis is the tuberculin skin test. Although numerous versions of the skin test are available, typically one of two preparations of tuberculin antigens are used: old tuberculin (OT), or purified protein derivative (PPD). The antigen preparation is either injected into the skin intradermally, or is topically applied and is then invasively transported into the skin with the use of a multiprong inoculator (Tine test). Several problems exist with the skin test diagnosis method. For example, the Tine test is not generally recommended because the amount of antigen injected into the intradermal layer cannot be accurately controlled. (See Murray et al. Medical Microbiology, The C.V. Mosby Company 219-230 (1990)).

Although the tuberculin skin tests are widely used, they typically require two to three days to generate results, and many times, the results are inaccurate since false positives are sometimes seen in subjects who have been exposed to mycobacteria, but are healthy. In addition, instances of mis-diagnosis are frequent since a positive result is observed not only in active TB patients, but also in persons vaccinated with Bacille Calmette-Guerin (BCG), and those who had been infected with mycobacteria, but have not developed the disease. It is hard therefore, to distinguish active TB patients from the others, such as household TB contacts, by the tuberculin skin test. Additionally, the tuberculin test often produces a cross-reaction in those individuals who were infected with mycobacteria other than M. tuberculosis (MOTT). Therefore, diagnosis using the skin tests currently available is frequently subject to error and inaccuracies.

The standard treatment for tuberculosis caused by drug-sensitive organisms is a six-month regimen consisting of four drugs given for two months, followed by two drugs given for four months. The two most important drugs, given throughout the six-month course of therapy, are isoniazid and rifampin. Although the regimen is relatively simple, its administration is quite complicated. Daily ingestion of eight or nine pills is often required during the first phase of therapy; a daunting and confusing prospect. Even severely ill patients are often symptom free within a few weeks, and nearly all appear to be cured within a few months. If the treatment is not continued to completion, however, the patient may experience a relapse, and the relapse rate for patients who do not continue treatment to completion is high. A variety of forms of patient-centered care are used to promote adherence with therapy. The most effective way of ensuring that patients are taking their medication is to use directly observed therapy, which involves having a member of the health care team observe the patient take each dose of each drug. Directly observed therapy can be provided in the clinic, the patient's residence, or any mutually agreed upon site. Nearly all patients who have tuberculosis caused by drug-sensitive organisms, and who complete therapy will be cured, and the risk of relapse is very low (“Ending Neglect: The Elimination of Tuberculosis in the United States” ed. L. Geiter Committee on the Elimination of Tuberculosis in the United States Division of Health Promotion and Disease Prevention, Institute of Medicine. Unpublished.)

Although the FDA approved a medication that combines the three main drugs (isoniazid, rifampin, and pyrazinamide) used to treat tuberculosis into one pill. Thereby reducing the number of pills a patient has to take each day and making it impossible for the patient to take only one of the three medications, a common path to the development of MDR-TB, there is still a need in the art to treat tuberculosis, especially in those cases wherein the tuberculosis strain is drug resistant.

What is needed are effective therapeutic regimens that include improved vaccination and treatment protocols. Currently available therapeutics are no longer consistently effective as a result of the problems with treatment compliance, and these compliance problems contribute to the development of drug resistant mycobacterial strains.

Mycobacterial infections, such as those causing tuberculosis, once thought to be declining in occurrence, have rebounded, and again constitute a serious health threat. Tuberculosis (TB) is the cause of the largest number of human deaths attributed to a single etiologic agent with two to three million people infected with tuberculosis dying each year. Areas where humans are crowded together or living in substandard housing, are increasingly found to have persons affected with mycobacteria. Individuals who are immunocompromised are at great risk of being infected with mycobacteria and dying from such infection. In addition, the emergence of drug-resistant strains of mycobacteria has led to treatment problems of such infected persons

Many people who are infected with mycobacteria are poor, or live in areas with inadequate healthcare facilities. As a result of various obstacles (economical, education levels, etc.), many of these individuals are unable to comply with the prescribed therapeutic regimens. Ultimately, persistent non-compliance by these and other individuals results in the prevalence of disease. This noncompliance is frequently compounded by the emergence of drug-resistant strains of mycobacteria. Effective compositions and vaccines that target various strains of mycobacteria are necessary to bring the increasing number of tuberculosis cases under control.

Chemotherapy is a standard treatment for tuberculosis. Some current chemotherapy treatments require the use of three or four drugs, in combination, administered daily for two months, or administered biweekly for four to twelve months. Table 1 lists several treatment schedules for standard tuberculosis drug regimens.

TABLE 1
Treatment Schedules for Standard TB Drug Regimens.

| | INDUCTION | | | | |
|---|---|---|---|---|---|
| STANDARD | PHASE | | | CONTINUATION | |
| DRUG | Dosing | | | PHASE | |
| REGIMEN | Schedule | DURATION | DRUG | Dosing Schedule | DURATION |
| Isoniazid | Daily, DOT | 8 weeks | Isoniazid | 2/week, DOT | 16 weeks |
| Rifampicin | Daily, DOT | 8 weeks | Rifampicin | 2/week, DOT | 16 weeks |
| Pyrazinamide | Daily, DOT | 8 weeks | | | |
| Ethambutol or | Daily, DOT | 8 weeks | | | |
| Streptomycin | | | | | |

Decades of misuse of existing antibiotics and poor compliance with prolong and complex therapeutic regimens has led to mutations of the mycobacterium tuberculosis and has created an epidemic of drug resistance that threatens tuberculosis control world wide. The vast majority of currently prescribed drugs, including the front line drugs, such as isoniazid, rifampin, pyrazinamide, ethambutol and streptomycin were developed from the 1950s to the 1970s.

Consequently, the treatments of drug-resistant M. tuberculosis strains, and latent tuberculosis infections, require new anti-tuberculosis drugs that provide highly effective treatments, and shortened and simplified tuberculosis chemotherapies.

U.S. Patent Application Publication No. 2006/0148904, U.S. Patent Application Publication No. 2006/0020041, and U.S. Pat. No. 6,951,961 all to Protopopova et al., describe substituted ethylene diamine compounds of the general structure

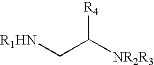

### Substituted Ethylene Diamine

which are prepared by a modular approach using primary and secondary amines as building blocks, and coupling the amine moieties with an ethylene linker building block. The groups R₁, R₂, and R₃ of the ethylene diamine compounds are independently H, alkyl; aryl; alkenyl; alkynyl; aralkyl; aralkenyl; aralkynyl; cycloalkyl; cycloalkenyl; heteroalkyl; heteroaryl; halide; alkoxy; aryloxy; alkylthio; arylthio; silyl; siloxy; a disulfide group; a urea group; amino; and the like; and R₄ is H, alkyl or aryl, but R₄ can also constitute alkenyl, alkynyl, aralkyl, aralkenyl, aralkynyl, cycloalkyl, cycloalkenyl, and the like

Capuramycin, a naturally occurring nucleoside antibiotic produced by Streptomyces griseus, inhibits bacterial translocase I, an enzyme essential in peptidoglycan biosynthesis of all bacteria. However, capuramycin has a narrow spectrum of activity and works best on mycobacteria. U.S. Pat. Nos. 6,472,384 and 6,844,173 both to Inukai et al., and U.S. Pat. No. 7,157,442 to Hotoda et al., all of which are incorporated herein by reference in their entirety, describe analogs and derivatives of natural capuramycin. In particular, esters, ether and N-alkylcarbamoyl derivatives were prepared and evaluated for activity against various mycobacterial agents. Several derivatives were found that exhibited potent antibacterial activity against Mycobacterium tuberculosis, Mycobacterium smegmatis and Mycobacterium avium in vitro. Capuramycin and the capuramycin derivatives have limited water solubility, which translates to lower bioavailability and limits the potential of these potent compounds. The limited bioavailability of the compounds results in lower efficacy in vivo and presents a challenge for the development of improved drugs for the treatment of tuberculosis and other mycobacterial infections using capuramycin ad derivatives of capuramycin. Therefore, there is a need to develop improved capuramycin derivatives with better aqueous solubility and bioavailability. There is also a need to develop improved compositions and formulations of capuramycin and capuramycin derivatives that exhibit better bioavailability.

Clearly effective therapeutic regimens that include improved vaccination and treatment protocols are needed. A therapeutic vaccine that would prevent the onset of tuberculosis, and therefore eliminate the need for therapy is desirable. Although currently available therapeutics such as ethambutol are effective, the emergence of drug resistant strains has necessitated new formulations and compositions that are more versatile than ethambutol. Currently available therapeutics are no longer consistently effective as a result of the problems with treatment compliance, lending to the development of drug resistant mycobacterial strains. What is needed are new anti-tubercular drugs that provide highly effective treatment, and shorten or simplify tuberculosis chemotherapy.

### SUMMARY OF THE INVENTION

Provided herein are new amino acid and modified derivatives of capuramycin (CM) (i.e. PEGylated) analogues of Formulae Ia, Ib, IIa and IIb. The present invention also provides insoluble particulate formulations, such as micellular, liposomal and nanoparticle formulations comprising capuramycin and capuramycin analogues of Formulae I, Ia, Ib, I-A, II, IIa or IIb, and a formulation comprising the compounds in combination with a PEGylated vitamin E derivative. The inventive formulations exhibit improved solubility, bioavailability and efficacy in vivo for the treatment and prevention of a disease caused by microorganisms.

Also provided herein are methods for the treatment of an infectious disease, including a mycobacterial disease, comprising administering to a patient in need of treatment an effective amount of an amino acid or PEG derivative of capuramycin or a capuramycin analogue of Formulae Ia, IIa, Ib or IIb or a formulation comprising a capuramycin analogue of Formulae I, Ia, Ib, I-A, II, IIa or IIb in combination with a PEGylated compound, including a PEGylated vitamin E derivative, or a formulation of the compounds in combination with an insoluble particulate carrier. Insoluble particulate carriers include micelles, liposomes and polymer nanoparticle carriers. In addition, provided herein are methods and compositions comprising capuramycin or a capuramycin derivative of Formulae I, Ia, Ib, I-A, II, IIa or IIb in combination with another active agent including, but not limited to, an anti mycobacterial agent such as ethambutol (EMB), rifampicin (RIF), isoniazid (INH), pyrazinamide, moxifloxacin (MOXI), streptomycin (SM), clarithromycin (CLA), amikacin (AMK), or the ethylenediamine compound SQ109 (see U.S. Pat. No. 6,951,961) and the like, for the treatment of an infectious disease, including a mycobacterial disease. Compositions comprising certain capuramycin analogues in combination with other anti-tuberculosis active agents were found to have synergistic efficacy in the treatment of mycobacterial infections, including M. smegmatis, M. tuberculosis, M. abscessus and M. avium.

In one embodiment, the invention provides an amino acid derivative of capuramycin or a capuramycin analogue of Formula Ia, or a pharmaceutically acceptable ether or ester thereof:

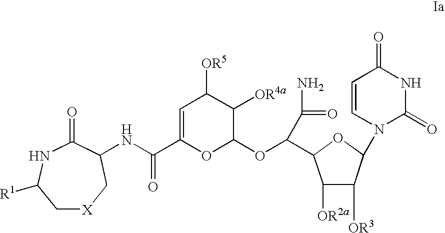

wherein R¹ is H or methyl; X is CH₂ or S; R^{4a} is H, a protecting group for a hydroxy group, or a residue of an amino acid; and R⁵, R^{2a} and R³ are independently H, methyl, alkanoyl, a protecting group for a hydroxy group, or a residue of an amino acid; wherein at least one of R⁵, R^{2a}, R³ or R^{4a} is a residue of an amino acid.

In other embodiments, the invention provides the amino acid derivatives of capuramycin analogues shown in Table 2 below.

In another aspect of the invention, PEGylated derivatives of capuramycin or capuramycin analogues of Formula Ib, or a pharmaceutically acceptable ether or ester thereof are provided:

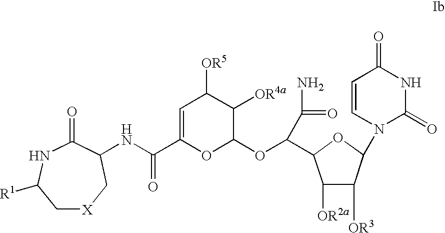

wherein R¹ is H or methyl; X is CH₂ or S; R^{4a} is H, a protecting group for a hydroxy group, or a group comprising a PEG residue; and R⁵, R^{2a} and R³ are independently H, methyl, alkanoyl, a protecting group for a hydroxy group, or a group that comprises a PEG residue; where at least one of R⁵, R^{4a}, R^{2a} or R³ is a group that comprises a PEG residue.

In a particular embodiment, the PEGylated derivative has the structure

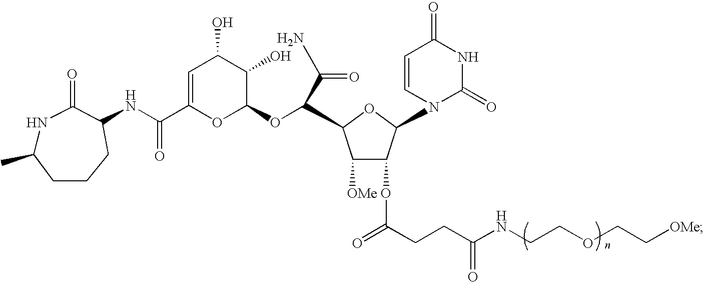

wherein n is 1-1000.

In another aspect of the invention, formulations comprising a capuramycin analogue of formula I-A or II, or pharmaceutically acceptable esters, ethers or N-alkylcarbamoyl derivatives thereof, and a hydrophobic organic compound that is derivatized with one or more a PEG residues are provided:

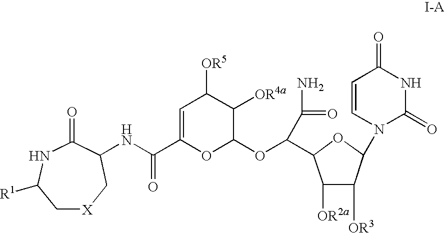

wherein R¹ is a hydrogen atom or a methyl group, R^{2a}, R³ or R⁵ are independently H, a protecting group for a hydroxy group, a methyl group, or an alkanoyl group; R^{4a} is H, or a protecting group for a hydroxy group, and X is a methylene group or a sulfur atom, or a pharmaceutically acceptable salt or prodrug thereof;

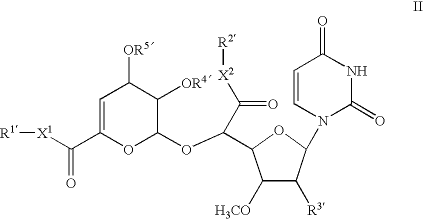

wherein X¹ represents an oxygen atom, a sulfur atom or —N(R⁶);

X² represents an oxygen atom, a sulfur atom or a —N(R⁷);

R^{1′} and R^{2′} are independently hydrogen; a methyl group; an optionally substituted aryl or heteroaryl group; an optionally substituted heterocyclic group; optionally substituted alkyl; an alkyl group which is substituted with one to three optionally substituted C₆-C₁₀ aryl groups, which may be the same or different; an alkyl group which is substituted with one to three optionally substituted heterocyclic groups, which may be the same or different; an optionally substituted alkenyl group; an optionally substituted alkynyl group; or a group of formula (a),

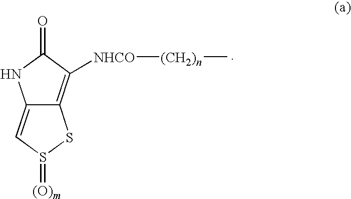

in which n is an integer from 1 to 20 and m is 0, 1 or 2; wherein each heterocyclic group has 1-4 nitrogen, sulfur or oxygen atoms;

R^{3′}, R^{4′} and R^{5′} are independently H, OH, alkanoyl, —O-alkanoyl, or a protecting group for a hydroxy group;

R⁶ is a hydrogen atom, a C₁-C₃ alkyl group, or R⁶, together with R^{1′} and the nitrogen atom to which they are attached, forms a 3- to 7-membered cyclic amine which may have a ring oxygen or sulfur atom; and

R⁷ is a hydrogen atom, a C₁-C₃ alkyl group, or R⁷, together with R^{2′} and the nitrogen atom to which they are attached, forms a 3- to 7-membered cyclic amine which may have a ring oxygen or sulfur atom;

wherein the aryl, heteroaryl, alkyl, alkenyl, or alkynyl groups are optionally substituted with acyl, alkyl, aryl, heterocyclic, halogen, hydroxyl, alkoxy, thiol, alkylthio, amino, alkyl or dialkylamino, nitro, cyano, carboxyl, carbamoyl, alkylenedioxy, aralkyloxy, CF₃ or —OCF₃.

In a preferred embodiment, the PEGylated compound comprises D-alpha-tocopheryl poly(ethylene glycol) 1000 succinate.

In another embodiment of the invention, formulations comprising a capuramycin analogue of formula I-A or II shown above, or pharmaceutically acceptable esters, ethers or N-alkylcarbamoyl derivatives thereof, in combination with a pharmaceutically acceptable carrier that is in the form of insoluble particulates are provided.

In one embodiment, the insoluble particulates comprise micelles. In another embodiment, the micelles comprise D-alpha-tocopheryl poly(ethylene glycol) 1000 succinate.

In still another embodiment, the insoluble particulates comprise liposomes.

In another embodiment, the insoluble particulates comprise biocompatible nanoparticle polymers.

In another embodiment, the formulations comprise the capuramycin analogues shown in Table 3 below.

In another aspect of the invention, methods for the treatment of a mycobacterial disease comprising administering to a host with a mycobacterial disease an amino acid derivative of formula Ia, or a PEGylated derivative of formula Ib are provided. In another embodiment, the invention provides methods for the treatment of a mycobacterial disease comprising administering a formulation comprising a capuramycin analogue of formulae I-A or II and a PEGylated compound. In still another embodiment, the invention provides a method for the treatment of a mycobacterial disease in a host, comprising administering to the host a formulation comprising a compound of formula I-A or II in combination with a pharmaceutically acceptable carrier that is in the form of insoluble particulates.

In another embodiment, the invention provides a method for the treatment of a mycobacterial disease in a host, comprising administering to the host with a mycobacterial disease a capuramycin analogue of formulae I-A or II, or pharmaceutically acceptable esters, ethers or N-alkylcarbamoyl derivatives thereof, in combination with another antimycobacterial active agent.

These and other objects, features and advantages of the present invention will become apparent after a review of the following detailed description of the disclosed embodiments and the appended claims.

### BRIEF DESCRIPTION OF THE FIGURES

FIG. 1 shows the intracellular activities of capuramycin analogues SQ641, SQ922 and SQ997 compared to the anti tubercular drug isoniazid (INH) after 4 days.

FIG. 2 shows the in vivo efficacy of capuramycin analogues SQ641, SQ922 and SQ997 in a mouse model after three weeks of therapy.

FIG. 3 shows micelles of TPGS and SQ641 at 200× magnification in a light microscope.

FIG. 4 shows macrophages unexposed or exposed to small or large micelles of TPGS and SQ641 at 200× magnification.

FIG. 5 shows the intracellular activity of various capuramycin analogue amino acid derivatives.

FIG. 6 shows the intracellular activity SQ641 in D-Tocopheryl polyethylene glycol 1000 succinate.

FIG. 7 shows the activity of SQ641 in combination with the MDR1 blocker cyclosporine (CsA).

FIG. 8 shows the activity of SQ641 in combination with the MDR1 blocker verapamil (VE).

FIG. 9 shows the activity of SQ641 in combination with the MRP1 blocker probenecid (PB).

FIG. 10 shows the activity of SQ641 in combination with the MRP1 blocker gemfibrozil (GF).

### DETAILED DESCRIPTION

The present invention may be understood more readily by reference to the following detailed description of the specific embodiments included herein. However, although the present invention has been described with reference to specific details of certain embodiments thereof, it is not intended that such details should be regarded as limitations upon the scope of the invention. The entire text of the references mentioned herein are hereby incorporated in their entireties by reference including U.S. Patent Application Publication No. 2006/0148904, U.S. Patent Application Publication No. 2006/0020041, U.S. Pat. No. 6,951,961 and U.S. Provisional Patent Application Ser. No. 60/381,220 filed May 17, 2002, all to Protopopova et al., U.S. Pat. Nos. 6,472,384 and 6,844,173 to Inukai et al., and U.S. Pat. No. 7,157,442 to Hotoda et al.

Provided herein are new capuramycin (CM) analogues and derivatives, novel formulations and compositions comprising capuramycin and capuramycin derivatives of Formulae I, Ia, Ib, I-A, II, IIa or IIb that exhibit improved solubility, bioavailability and efficacy in vivo for the treatment and prevention of a disease caused by microorganisms including, but not limited to, mycobacterial infections. Also provided herein are methods for the treatment of an infectious disease, including a mycobacterial disease, comprising administering an effective amount of the compounds of Formulae I, Ia, Ib, I-A, II, IIa or IIb in a liquid or particulate formulation to a patient in need of treatment. In addition, provided herein are methods and compositions comprising capuramycin or a capuramycin derivative of Formula I, Ia, Ib, I-A, II, IIa or IIb in combination with one or more additional active agents including, but not limited to, an anti mycobacterial agent, such as ethambutol (EMB), rifampicin (RIF), isoniazid (INH), pyrazinamide, moxifloxacin (MOXI), streptomycin (SM), clarithromycin (CLA), amikacin (AMK), or the ethylenediamine compound SQ109, and the like, for the treatment of an infectious disease, including a mycobacterial disease. Certain capuramycin analogues in combination with other anti-tuberculosis active agents were found to have synergistic efficacy in the treatment of certain mycobacterial infections, including M. smegmatis, M. tuberculosis, M. abscessus and M. avium.

In spite of the potent antibiotic properties of capuramycin and capuramycin analogues against Mycobacteria, the compounds are have limited aqueous solubility and are not absorbed well when delivered orally. Furthermore, capuramycin and capuramycin analogues do not typically concentrate in macrophages, which harbor Mycobacterium in infected animals and are an important target for treating the disease. It has been discovered that the limited bioavailability of these compounds is in part due to the activation of P-glycoprotein (P-gp) drug efflux from macrophages, resulting in low concentrations of the compounds in macrophages. The present invention provides derivatives of capuramycin and capuramycin analogues with improved aqueous solubility and bioavailability, and inventive formulations of capuramycin and capuramycin analogues that exhibit improved bioavailability and improved in vivo efficacy.

### Amino Acid Derivatives of Capuramycin

One approach to decrease the P-gp mediated efflux of the compounds of the invention and induce uptake of capuramycin and capuramycin analogues is to prepare amino acid derivatives of the active compounds. Amino acid transporters are expressed in almost all living cells and are responsible for the adsorption of amino acids; it has been postulated that prodrugs of drugs coupled to amino acid residues may improve the adsorption of drugs and avoid the P-gp mediated efflux of the drugs. For example, a valine conjugate was used to overcome the P-gp mediated efflux of quinidine (Hu et al., 2004, IDrugs, 7(8), 736-42). In addition, amino acid conjugates of compounds with low aqueous solubility are often more soluble than the parent compounds and are capable of forming salts.

The inventors herein have discovered that certain amino acid derivatives of capuramycin analogues of the invention decrease the interaction of the compounds with P-gp and exhibit increased intracellular activity. For example, the undecanoic acid amino acid derivative of capuramycin analogue SQ641 was found to have increased intracellular activity against M. tuberculosis (MTB) compared with underivatized SQ641. Though amino acid conjugation for improved drug delivery has been devised for certain drugs, identifying the appropriate amino acid for a specific compound requires significant experimentation. Furthermore, determining the relevant chemical parameters, and defining the optimal chemistry requires significant experimentation.

In one embodiment, amino acid derivatives of Formulae Ia or IIa are provided that have improved aqueous solubility compared to the parent compound. The amino acid(s) in any of the embodiments of the invention described herein may be naturally occurring or synthetic amino acids. The amino acids may be in the D or L stereoisomeric form or may exist as a D, L mixture. For example the 20 naturally occurring α-amino acids in the L-configuration are encompassed by the invention as well as α-amino acids in the D-configuration. Synthetic amino acids in either stereoisomeric form are also encompassed.

In one embodiment, an amino acid derivative of the compound of Formula Ia, or a pharmaceutically acceptable ether or ester thereof, is provided

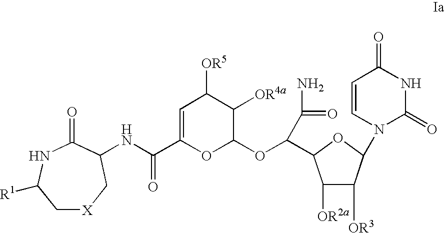

wherein R¹ is H or methyl; X is CH₂ or S; R^{4a} is H, a protecting group for a hydroxy group, or a residue of an amino acid; and R⁵, R^{2a} and R³ are independently H, methyl, alkanoyl, a protecting group for a hydroxy group, or a residue of an amino acid; wherein at least one of R⁵, R^{2a}, R³ or R^{4a} is the residue of an amino acid. In another embodiment, R⁵, R^{2a} or R³ are independently H, methyl, alkanoyl, or a residue of an amino acid which forms an ester bond with the hydroxy group of the compound. In still another embodiment, R^{4a} or R⁵ is an ester of amino acid residue.

In another embodiment, an amino acid derivative of Formula IIa, or a pharmaceutically acceptable ether or ester thereof, is provided

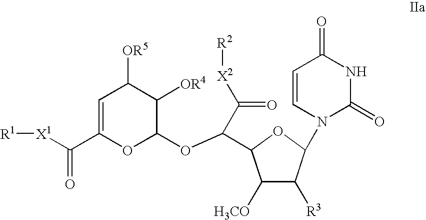

wherein X¹ represents an oxygen atom, a sulfur atom or a group of formula —N(R⁶)— (in which R⁶ is a hydrogen atom, a C₁-C₃ alkyl group, or R⁶, together with R¹ and the nitrogen atom to which they are attached, forms a 3- to 7-membered cyclic amine which may have a ring oxygen or sulfur atom);

X² represents an oxygen atom, a sulfur atom or a group of formula —N(R⁷)— (in which R⁷ is a hydrogen atom, a C₁-C₃ alkyl group, or R⁷, together with R² and the nitrogen atom to which they are attached, forms a 3- to 7-membered cyclic amine which may have a ring oxygen or sulfur atom);

R¹ and R² are independently hydrogen; an optionally substituted C₆-C₁₀ aryl or heteroaryl group; an optionally substituted heterocyclic group; optionally substituted alkyl, an alkyl group which is substituted with one to three optionally substituted C₆-C₁₀ aryl groups, which may be the same or different; an alkyl group which is substituted with one to three optionally substituted heterocyclic groups, which may be the same or different; optionally substituted alkenyl; optionally substituted alkynyl; a residue or an amino acid; or a group of formula (a), in which n is an integer from 1 to 20 and m is 0, 1 or 2; wherein each heterocyclic group has 1-4 nitrogen, sulfur or oxygen atoms; and wherein aryl, heteroaryl, alkyl, alkenyl, or alkynyl are optionally substituted with acyl, alkyl, aryl, heterocyclic, halogen, hydroxyl, alkoxy, thiol, alkylthio, amino, alkyl or dialkylamino, nitro, cyano, carboxyl, carbamoyl, alkylenedioxy, aralkyloxy, CF₃ or —OCF₃; and

R³, R⁴ and R⁵ are independently H, OH, —O-alkanoyl, alkanoyl, a protecting group for a hydroxy group, or a residue of an amino acid; wherein at least one of R³, R⁴ or R⁵ is a residue of an amino acid.

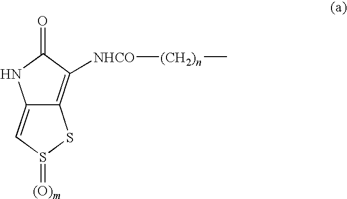

In another embodiment, R³ and/or R⁴ and/or R⁵ are independently the residue of an amino acid which is bonded to the compound by an ester bond to the carboxyl group. In another embodiment, R³ and/or R⁴ and/or R⁵ are independently the residue of a naturally occurring amino acid in the D- or L-configuration, which forms an ester bond with the compound. In another embodiment, R³ is a C₁-C₁₂ —O-alkanoyl group.

In still another embodiment, R³ and/or R⁴ and/or R⁵ are the residue of a C₂-C₂₄ aminoalkanoic acid which forms an ester with the compound. In some embodiments, the amino acid residue comprises an α-amino acid, including a D- or L-α-amino acid. In other embodiments, the amino acid residue comprises alkyl amino acids, including but not limited to, aminoalkanoic acids, where the alkyl group includes but is not limited to C₁-C₂₀ alkyl groups, such as aminooctanoic acid, aminononanoic acid, aminopentanoic acid, aminoundecanoic acid, and the like. In another embodiment, R³ and/or R⁴ and/or R⁵ are a heterocyclic amino acid such as isonipecotic acid and the like. In another embodiment, R³ is the residue of an aromatic or heteroaromatic amino acid including, but not limited to, aminobenzoic acid, 4-aminophenylacetic acid, anthranilic acid, 3-amino-2-naphthoic acid, nicotinic acid, isonicotinic acid and the like.

Scheme 1 below shows a non-limiting example of the synthesis of amino acid derivatives of capuramycin analogues. Starting from the capuramycin analogue SQ997, the diol functionality is protected as the ketal using acetone dimethylacetal under acidic conditions. The protected compound is then activated with dicycohexylcarbodiimide (DCC) and reacted with a Boc-protected amino acid in the presence of 4-dimethylaminopyridine (DMAP) as catalyst. The product is deprotected to form the desired amino acid derivative.

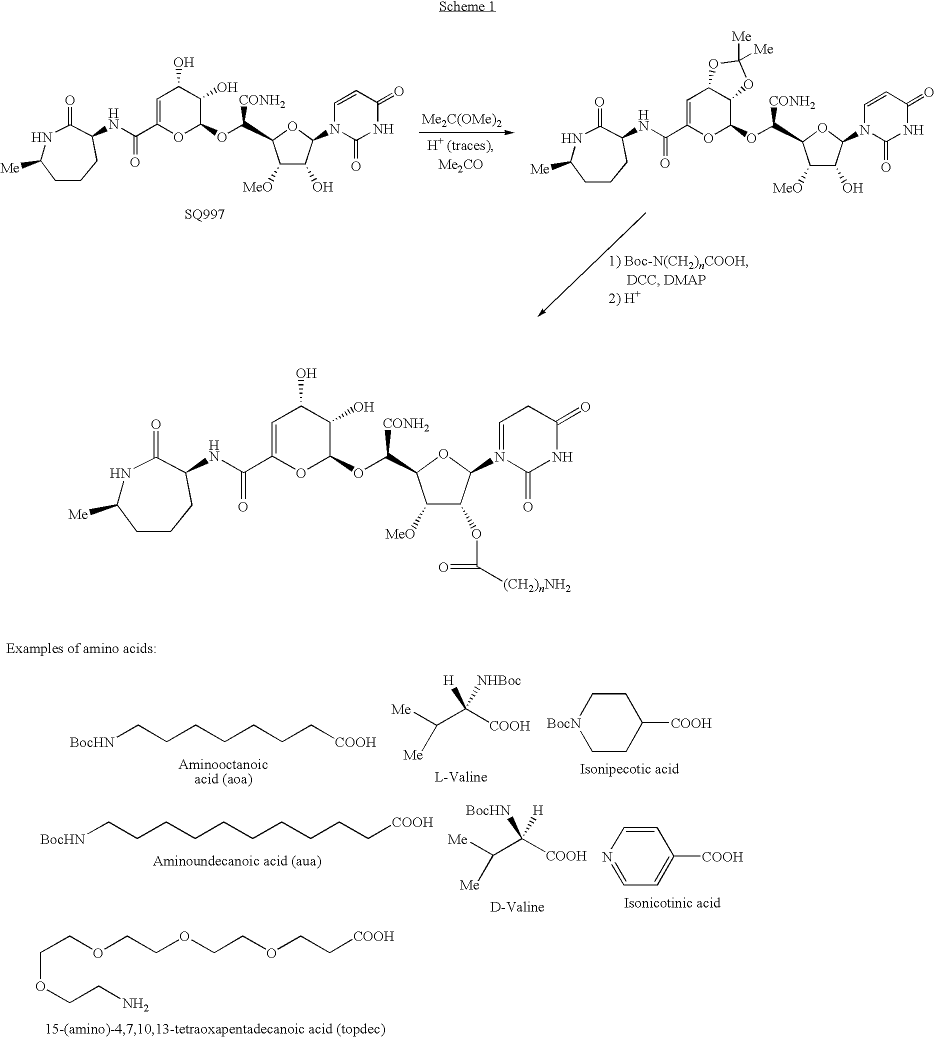

In some embodiments of the invention, capuramycin or a capuramycin analogue may be derivatized with two or more amino acid groups by reacting two or more hydroxyl groups or other nucleophilic sites on the molecule with an activated amino acid derivative. The two or more amino acids may be the same or different. For example, derivatives with two different amino acid groups may be formed by reacting a capuramycin analogue with a first activated amino acid group and then reacting the mono-amino acid intermediate with a second activated amino acid group. The first product may be isolated and purified, if desired, before reacting the intermediate with a second activated amino acid.

It will be apparent to those skilled in the art that alternative reagents and conditions may be used for the synthesis of capuramycin analogue amino acid derivatives. Many reagents are known in the art for the preparation of esters by the reaction of an activated carbonyl derivative with a hydroxy group. For example, it will be apparent that protected acyl halide derivatives of amino acids or other activated carbonyl reagents may be used in place of a protected amino acid with DCC and DMAP.

Alternate coupling reagents known in the art may be used to effect the ester bond formation. For example, a variety of peptide coupling reagents well known in the art are used to activate carboxyl groups in-situ to react with amino groups of protected amino acids to form peptide bonds. Examples of carboxyl activating groups include, but are not limited to, carbodiimide reagents, phosphonium reagents such as benzotriazolyloxy-tris-(dimethylamino)phosphonium hexafluorophosphate (BOP) and the like, uronium or carbonium reagents such as O-(benzotriazol-1-yl)-N,N,N′,N′-tetramethyluronium hexafluorophosphate (HBTU), benzotriazol-1-yl-oxy-tripyrrolidinophosphonium hexafluorophosphate (PyBOP) and the like; 1-ethoxycarbonyl-2-ethoxy-1,2-dihydroqunoline (EEDQ); 1-methyl-2-chloropyridinium iodide (Muikaiyama's reagent) and the like.

In other embodiments, the ester may be formed by trans-esterification of another ester group, including active esters such as a p-nitrophenyl ester, a pentafluorophenylester, an N-hydroxysuccinimidyl ester, a 1-hydroxybenzotriazolyl ester, and the like. An acyl azide derivative of an amino acid may also be used to form the ester bond.

In another embodiment, the ester may also be formed by reaction of a hydroxy group with a symmetric or mixed anhydride comprising the desired amino acid. Catalysts such as 4-dimethylaminopyridine (DMAP) and the like may be used to facilitate the ester formation.

In another embodiment, the ester bond may be formed under Mitsunobu reaction conditions by treating a capuramycin analogue and a protected amino acid with diethylazodicarboxylate (DEAD) and triphenylphosphine (Mitsunobu, O.; Yamada, Y. Bull Chem. Soc. Japan 1967, 40, 2380-2382). Alternate reagents such as polymer-bound phosphines and polymer-bound DEAD may be used in the Mitsunobu reaction.

In another embodiment, amino acid derivatives of capuramycin analogues may be formed by reacting an activated amino acid with one or both hydroxy groups on the dihydropyran ring or another nucleophilic site on the capuramycin analogue.

It will be apparent to one of skill in the art that different protecting groups may be used for the preparation of amino acid derivatives of capuramycin analogues as long as they are compatible with the reaction conditions and provide the necessary stability. A wide variety of functional protecting groups are taught by Greene et al. Protective Groups in Organic Synthesis, John Wiley and Sons, Third Edition, 1999.

Scheme 2 below shows the preparation of amino acid derivatives of SQ641, a capuramycin analogue in which the hydroxy groups on the nucleoside sugar ring are blocked. The amino acid derivatives are formed by reacting the free hydroxyl groups on the dihydropyran ring.

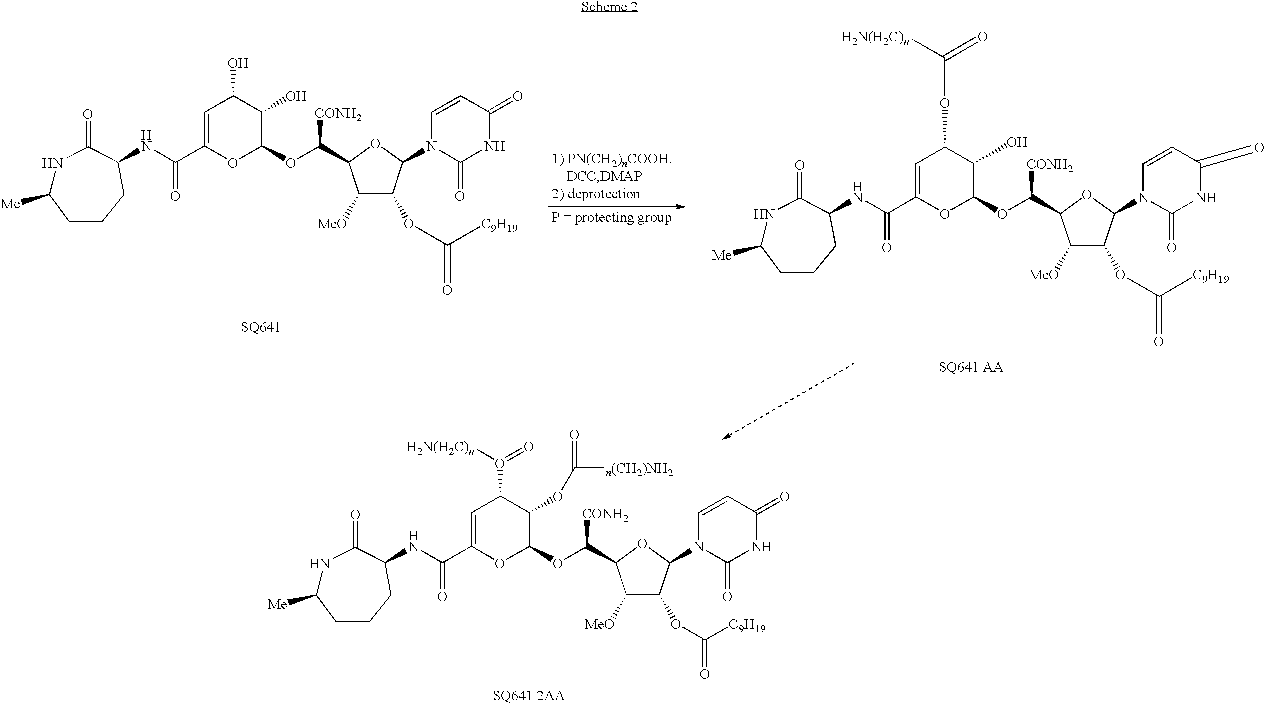

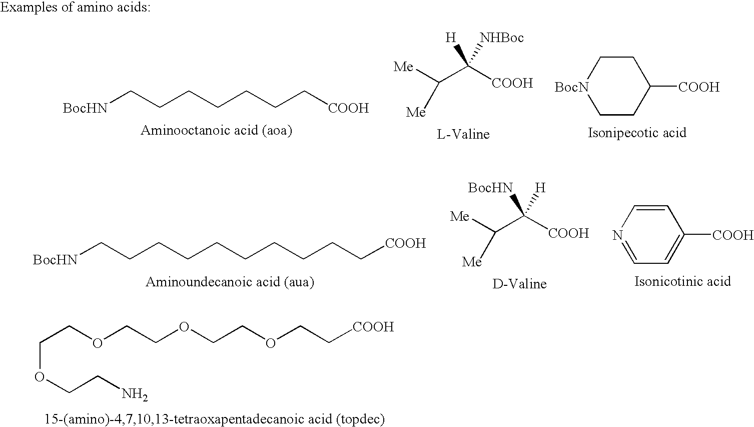

In specific embodiments, the specific amino acid derivatives in Table 2 below are provided.

TABLE 2

| Compound | Structure |
|---|---|
| SQ997-aoa | 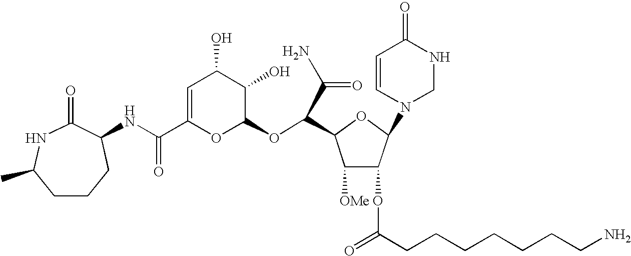 |
| SQ 997-aua | 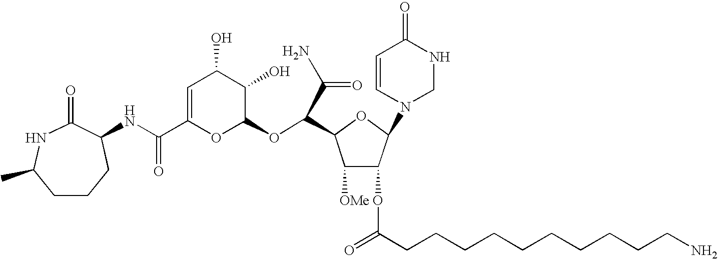 |
| SQ 997- isonipecotic acid | 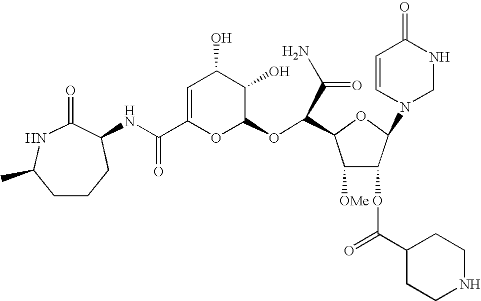 |
| SQ 997- isonicotinic acid | 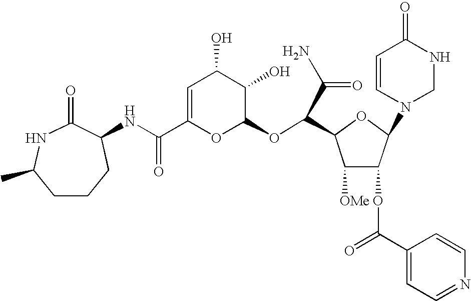 |
| SQ 997-L-Val | 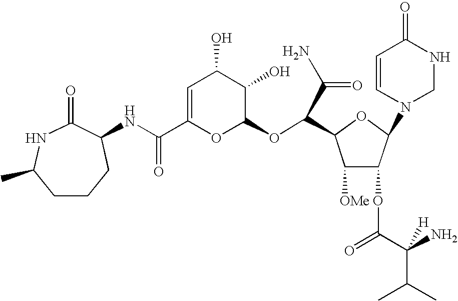 |
| SQ 997-D-Val | 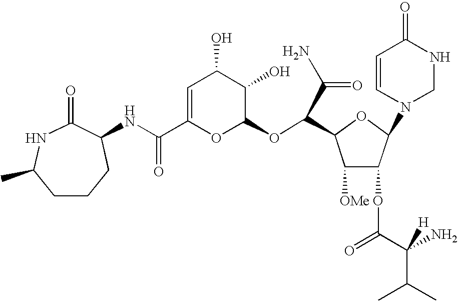 |
| SQ641-aua | 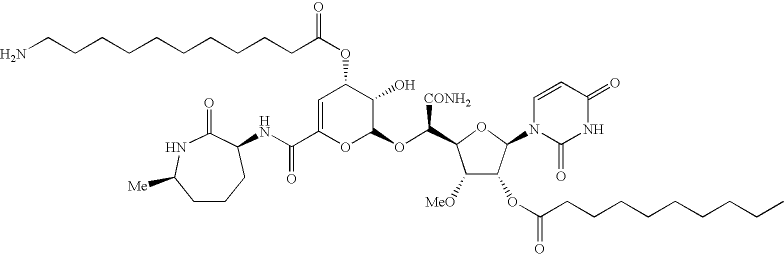 |
| SQ641-topdec | 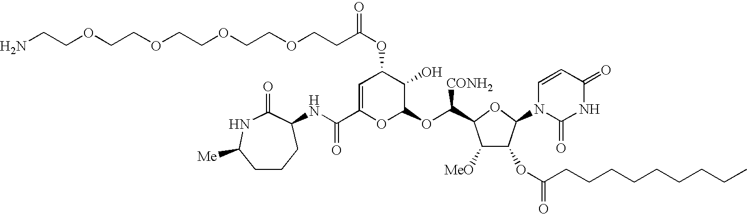 |
| SQ641-2topdec | 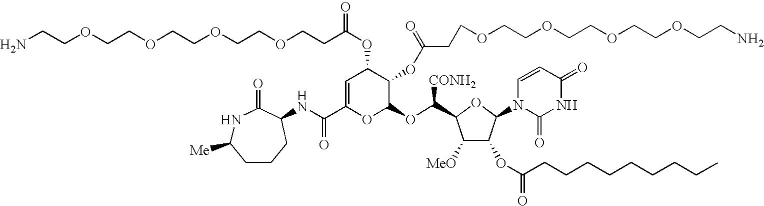 |

Polymer Conjugates of Capuramycin Analogues

In another embodiment, novel polymer conjugates of capuramycin or capuramycin analogues are provided which exhibit improved aqueous solubility and bioavailability. The polymer conjugates of the invention are prepared by derivatizing the compounds of Formulae I, I-A or II with desired polymer components. Surprisingly, it has been shown that polymer conjugates of capuramycin analogues exhibit reduced P-gp mediated drug efflux and improved intracellular activity compared to the parent drugs. Capuramycin analogues have not been previously conjugated to polymers and it was not known or predicted that polymer-conjugated capuramycin analogues would offer improved bioavailability and intracellular efficacy compared to the parent drug. Furthermore, the surprising impact of polymer conjugates of the capuramycin analogues on the P-gp mediated drug efflux was not predictable.

The polymer portion of the polymer conjugate may comprise, for example, polyethylene glycol (“PEG”), polypropylene glycol (“PPG”), polyoxyethylated glycerol (“POG”) and other polyoxyethylated polyols, polyvinyl alcohol (“PVA) and other polyalkylene oxides, polyoxyethylated sorbitol, polyoxyethylated glucose or other biologically compatible polymers. In other embodiments, the polymer is a polyethylene/polypropylene copolymer including, but not limited to, a poloxamer. In another embodiment, the polymer is a dextran polymer. The polymer can be a homopolymer, a random or block copolymer, a terpolymer based on the monomers listed above, straight chain or branched, substituted or unsubstituted. The polymeric portion can be of any length or molecular weight but these characteristics can affect the biological properties. Polymer average molecular weights particularly useful for decreasing clearance rates in pharmaceutical applications are in the range of 500 to 35,000 Daltons. Thus, one skilled in the art can vary the length of the polymer to optimize or confer the desired biological activity.

In one preferred embodiment, the polymer is a PEG polymer. In another embodiment, the polymer is a block co-polymer of polyethylene glycol and polypropylene glycol known as poloxamers or by the trade name Pluronic® (BASF, Germany). The covalent attachment of PEG to a drug can “mask” the agent from the host's immune system, reducing immunogenicity and antigenicity, increase the hydrodynamic size (size in solution) of the agent which prolongs its circulatory time by reducing renal clearance. PEGylation can also provide water solubility to hydrophobic compounds. Decreased clearance can lead to increased efficiency over the non-PEGylated material. See, for example, Conforti et al., Pharm. Research Commun. vol. 19, pg. 287 (1987) and Katre et al., Proc. Natl. Acad. Sci. U.S.A. vol. 84, pg. 1487 (1987).

In addition, PEGylation can decrease protein aggregation (Suzuki et al., Biochem. Biophys. Acta vol. 788, pg. 248 (1984)), alter protein immunogenicity (Abuchowski et al., J. Biol. Chem. vol. 252 pg. 3582 (1977)), and increase protein solubility as described, for example, in PCT Publication No. WO 92/16221, which is hereby incorporated by reference in its entirety.

The clinical value of PEGylation is now well established. ADAGEN® (PEG-bovine adenosine deaminase) manufactured by Enzon Pharmaceuticals, Inc. (Bridgewater, N.J.), for the treatment of X-linked severe combined immunogenicity, was the first PEGylated protein approved by FDA in March 1990, to enter the market. Various other PEGylated pharmaceuticals have been approved and since the initial approval of ADAGEN®. For example, PEGASYS® (Hoffman-LaRoche, Basel Switzerland) and PEG-Intron® (Schering-Plough, Kenilworth, N.J.) are approved PEGylated alpha-interferons used to treat hepatitis C. Oncaspar® is PEGylated L-asparaginase used for the treatment of acute lymphoblastic leukemia and Doxil® is a PEGylated liposome formulation containing doxorucin for the treatment of cancer.

The PEGylation of active agents is well known and there are many commercially available PEG reagents with functional groups that may be used to covalently attach PEG groups of varying size to compounds. Any known method of covalently attaching a PEG group to the compounds of Formula I, I-A or II may be used in the present invention. For example, PEG polymers with terminal reactive groups are available that may be utilized to form a covalent bond with a reactive site, such as a hydroxy or amino group, of the compounds of Formula I, I-A or II.

In addition, PEG polymers may be synthetically modified to form reactive species that can be used to form PEGylated compounds. U.S. Pat. No. 4,179,37 to Davis, which is incorporated by reference in its entirety, describes the PEGylation of peptides and polypeptides by modifying a terminal end of a PEG polymer and reacting the modified polymer with a peptide or polypeptide. U.S. Pat. No. 5,122,614 to Zalipsky et al., which is incorporated by reference in its entirety, describes that PEG molecules activated with an oxycarbonyl-N-dicarboximide functional group that can be attached under aqueous, basic conditions by a urethane linkage to the amine group of a polypeptide. Activated PEG-N-succinimide carbonate is said to form stable, hydrolysis-resistant urethane linkages with amine groups. European Patent No. EP 473 084, which is incorporated by reference herein in its entirety, describes PEG reagents comprising an aryl ring with two PEG chains and a carboxylate group, that may be used to PEGylate compounds. U.S. Pat. No. 6,552,170 to Thompson et al., which is incorporated herein by reference in its entirety, describes biologically active molecules containing a thiol group that are conjugated with PEG groups. Of course other PEG derivatives with different reactive moieties may be used to form a covalent bond with the compounds.

In some embodiments, groups comprising PEG residues include linkers with functional groups that may be activated and reacted to nucleophilic sites on the molecule. For example, in some embodiments, groups that comprise PEG residues include a terminal carboxyl group that may be activated to form an ester with a hydroxy group on capuramycin or capuramycin analogues of the invention. In another embodiment, the groups comprising a PEG residue comprise a linker group that comprises a reactive functional group or that can be activated to react with a nucleophilic site on the molecule, including a carboxyl group or the like. In one embodiment, the linker group comprises a dicarboxylic acid or a derivative of a dicarboxylic acid. Typically, one end of the liner is connected to a PEG residue and the other end of the linker is reacted with a nucleophilic site on capuramycin or the capuramycin analogues to form the PEGylated derivatives. In another embodiment the linker moiety comprises succcinic acid, glutaric acid, methylglutaric acid, malonic acid, adipic acid, or maleic acid, or derivatives of dicarboxylic acids, such as amides, esters and the like. In other embodiments, the linker is a bifunctional linker that comprises a carboxyl group or a derivative of a carboxyl group and another functional group, such as an amino group, a hydroxy group, a halogen, and the like. In another embodiment, the linker comprises an amino acid, including naturally occurring and synthetic amino acids. In still other embodiments, the linker comprises carbonate or urethane groups that bond the PEG residues to the linker or capuramycin or the capuramycin analogues to the linker.

In one embodiment, a hydroxy group of a compound of Formula I, I-A or II is reacted with a suitable activated polymer reagent, including a PEG reagent that contains a terminal carboxylate group to form a polymer conjugate. In another embodiment, a PEGylated derivative of Formula Ib, or a pharmaceutically acceptable ether or ester thereof, is provided

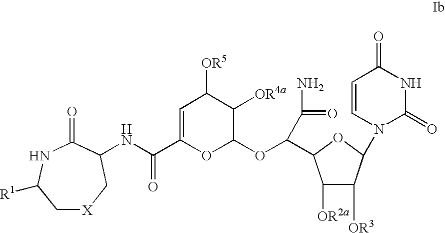

wherein R¹ is H or methyl; X is CH₂ or S; R^{4a} is H, a protecting group for a hydroxy group, or a group comprising a PEG residue; and R⁵, R^{2a} and R³ are independently H, methyl, alkanoyl, a protecting group for a hydroxy group, or a group that comprises a PEG residue; where at least one of R⁵, R^{4a}, R^{2a} or R³ is a group that comprises a PEG residue. In a preferred embodiment, groups comprising the PEG residues form an ester bond with one or more hydroxy groups of the compound. In a particular embodiment, R³ is a group that comprises PEG residue. In another embodiment, R⁵ is a group that comprises a PEG residue. In another embodiment, R^{2a} is a group that comprises a PEG residue. In still another embodiment, R^{2a} and/or R³ and/or R^{4a} and/or R⁵ are groups comprising a PEG residue. In one embodiment, the PEG residues are terminated by an alkyl group, such as methyl or ethyl. In another embodiment, the PEG residues are terminated by a hydroxy group.

In particular embodiments, PEGylated compounds of the formula Ib-1

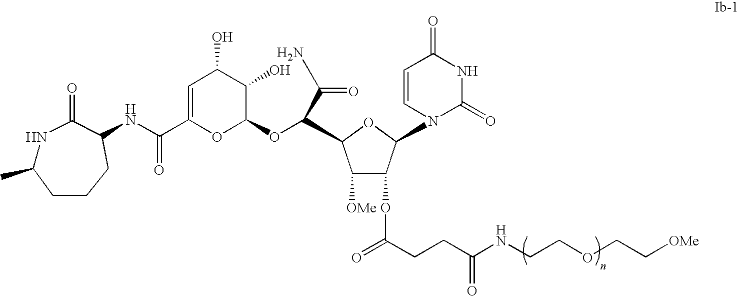

are provided, wherein n is 1-1000. In another embodiment, the PEGylated compound has a molecular weight of about 3 kDa or about 5 kDa. The PEGylated compounds of Formula Ib or IIb exhibit decreased affinity for P-gp mediated drug efflux and improved efficacy against mycobacterial agents in vitro.

In still another embodiment, a PEGylated derivative of Formula IIb, or a pharmaceutically acceptable ether or ester thereof, is provided.

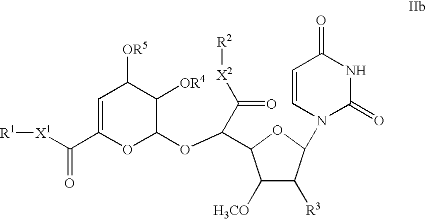

wherein X¹ represents an oxygen atom, a sulfur atom or a group of formula —N(R⁶)— (in which R⁶ is a hydrogen atom, a C₁-C₃ alkyl group, or R⁶, together with R¹ and the nitrogen atom to which they are attached, forms a 3- to 7-membered cyclic amine which may have a ring oxygen or sulfur atom);

X² represents an oxygen atom, a sulfur atom or a group of formula —N(R⁷)— (in which R⁷ is a hydrogen atom, a C₁-C₃ alkyl group, or R⁷, together with R² and the nitrogen atom to which they are attached, forms a 3- to 7-membered cyclic amine which may have a ring oxygen or sulfur atom);

R¹ and R² are independently hydrogen; an optionally substituted C₆-C₁₀ aryl or heteroaryl group; an optionally substituted heterocyclic group; optionally substituted alkyl, an alkyl group which is substituted with one to three optionally substituted C₆-C₁₀ aryl groups, which may be the same or different; an alkyl group which is substituted with one to three optionally substituted heterocyclic groups which may be the same or different; optionally substituted alkenyl; optionally substituted alkynyl; a group that comprises a PEG residue; or a group of formula (a), in which n is an integer from 1 to 20 and m is 0, 1 or 2; wherein each heterocyclic group has 1-4 nitrogen, sulfur or oxygen atoms; and wherein aryl, heteroaryl, alkyl, alkenyl, or alkynyl are optionally substituted with acyl, alkyl, aryl, heterocyclic, halogen, hydroxyl, alkoxy, thiol, alkylthio, amino, alkyl or dialkylamino, nitro, cyano, carboxyl, carbamoyl, alkylenedioxy, aralkyloxy, CF₃ or —OCF₃; and

R³, R⁴ and R⁵ are independently H, OH, —O-alkanoyl, alkanoyl, a protecting group for a hydroxy group, or a group comprising a PEG residue; wherein at least one of R³, R⁴ or R⁵ is a group that comprises a PEG residue. In a preferred embodiment, the group comprising a PEG residue forms an ester bond with one or more hydroxy groups of the compound.

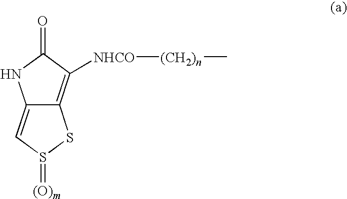

In another embodiment, capuramycin or a capuramycin analogue of formulae Ib or IIb are derivatized with a multiarmed PEG derivative. Multi-armed PEG reagents are known in the art and described in U.S. Pat. No. 5,932,462 to Harris et al., and U.S. Pat. No. 6,113,906 to Greenwald et al. which are hereby incorporated by reference in their entirety. In some embodiments, capuramycin or a capuramycin analogue of the invention is derivatized with a multi-armed PEG reagent that comprises a β-alanine liner moiety.

### Formulations

Novel liquid or solid particulate formulations of capuramycin and capuramycin analogues are provided herein which exhibit surprisingly improved solubility and/or bioavailability compared with standard formulations in aqueous carriers or solvents such as dimethylsulfoxide (DMSO). Although capuramycin analogues exhibit exquisite efficacy in vitro, it is a significant challenge to develop formulations that have sufficient solubility and bioavailability. The inventive formulations of the present invention achieve greatly improved bioavailability and solubility. The inventive capuramycin formulations include formulations with PEG-containing compounds, such as D-alpha-tocopheryl poly(ethylene glycol) 1000 succinate (TPGS 1000); formulations comprising insoluble particulate material, such as micelles and liposomes; and formulations comprising nanoparticle carriers.

In one embodiment of the present invention, novel formulations comprising a compound of Formula I are provided.

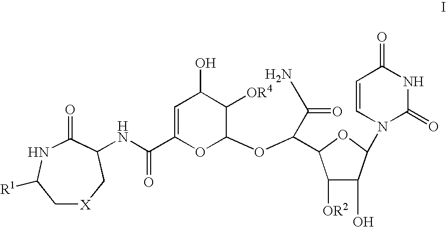

wherein R¹ is a methyl group, R² is a methyl group, R⁴ is hydrogen, and X is a methylene group; or

R¹ is a methyl group, R² is a hydrogen atom, R⁴ is hydrogen, and X is a methylene group; or

R¹ is a hydrogen atom, R² is a hydrogen atom, R⁴ is hydrogen, and X is a methylene group; or

R¹ is a methyl group, R² is a methyl group, R⁴ is hydrogen, and X is a sulfur atom, or a pharmaceutically acceptable salt or prodrug thereof.

In another embodiment, novel formulations comprising the compounds of Formulae Ia, Ib, IIa or IIb described above are provided.

In another embodiment, the present invention provides novel formulations comprising a compound of formula I-A, or a pharmaceutically acceptable ester, ether or N-alkylcarbamoyl derivative thereof:

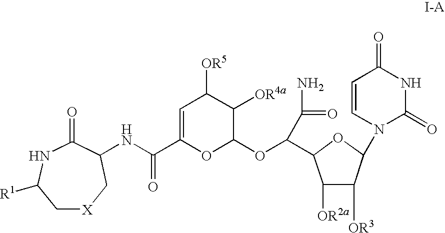

wherein R¹ is a hydrogen atom or a methyl group, R^{2a}, R³ or R⁵ are independently H, a protecting group for a hydroxy group, a methyl group, or an alkanoyl group; R^{4a} is a H, or a protecting group for a hydroxy group, and X is a methylene group or a sulfur atom, or a pharmaceutically acceptable salt or prodrug thereof.

In another embodiment, the present invention provides novel formulations comprising a compound of formula II, or a pharmaceutically acceptable ester, ether or N-alkylcarbamoyl derivative thereof:

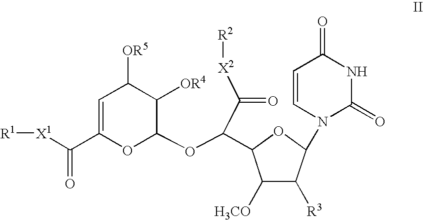

wherein X¹ represents an oxygen atom, a sulfur atom or a group of formula —N(R⁶)— (in which R⁶ is a hydrogen atom, a C₁-C₃ alkyl group, or R⁶, together with R¹ and the nitrogen atom to which they are attached, forms a 3- to 7-membered cyclic amine which may have a ring oxygen or sulfur atom);

X² represents an oxygen atom, a sulfur atom or a group of formula —N(R⁷)— (in which R⁷ is a hydrogen atom, a C₁-C₃ alkyl group, or R⁷, together with R² and the nitrogen atom to which they are attached, forms a 3- to 7-membered cyclic amine which may have a ring oxygen or sulfur atom);

R¹ and R² are independently hydrogen; optionally substituted aryl or heteroaryl group; an optionally substituted heterocyclic group; optionally substituted alkyl; an alkyl group which is substituted with one to three optionally substituted C₆-C₁₀ aryl groups, which may be the same or different; an alkyl group which is substituted with one to three optionally substituted heterocyclic groups which may be the same or different; an optionally substituted alkenyl group; an optionally substituted alkynyl group; or a group of formula (a), in which n is an integer from 1 to 20 and m is 0, 1 or 2; wherein each heterocyclic group has 1-4 nitrogen, sulfur or oxygen atoms; and

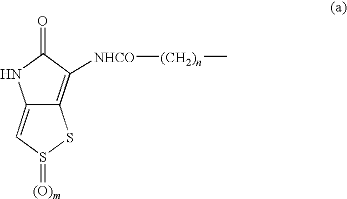

R³, R⁴ and R⁵ are independently H, OH, alkanoyl, —O-alkanoyl, or a protecting group for a hydroxy group;

wherein the aryl, heteroaryl, alkyl, alkenyl, or alkynyl groups are optionally substituted with acyl, alkyl, aryl, heterocyclic, halogen, hydroxyl, alkoxy, thiol, alkylthio, amino, alkyl or dialkylamino, nitro, cyano, carboxyl, carbamoyl, alkylenedioxy, aralkyloxy, CF₃ or —OCF₃.

All terms used herein are intended to have their ordinary meaning unless otherwise provided.

All ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Where numeral number or range is modified by the term “about,” it will be understood to embrace somewhat larger or smaller values than the indicated value to account for experimental errors inherent in the measurement and variability between different methodologies for measuring the value, as will be apparent to one skilled in the art.

The term “lower alkyl” refers to a C₁-C₆ alkyl group such as the methyl, ethyl, propyl, isopropyl, butyl, isobutyl, tert-butyl, pentyl and hexyl group.

The term “residue of an amino acid” refers to an amino acid group bonded to the compound by a bond to any suitable position of the amino acid. For example, an amino acid may be bonded to the compound by a bond to the oxygen of the carboxyl group of the amino acid or a bond to the carbonyl carbon of the amino acid or by a bond to the amino group of the amino acid.

In the above formulae, the protecting group of “protecting group for a hydroxy group” and “protected hydroxy group” and the like can be removed by a chemical procedure such as hydrogenolysis, hydrolysis, electrolysis or photolysis (hereinafter referred to as a general protecting group) or can be removed by biological method such as hydrolysis in vivo (with the proviso that it is not an ester residue group such as an acyl group). “The protecting group which can be removed by biological method such as hydrolysis in vivo” can be cleaved by biologically method such as hydrolysis in the human body to give a corresponding free acid or a salt thereof. Whether a compound has a protecting group removed in vivo is determined by detection of a corresponding parent compound or a pharmaceutically acceptable salt thereof in the body fluid of a rat or mouse to which it is administered by intravenous injection.

A general protecting group is selected from the group consisting of: a tetrahydropyranyl or a tetrahydrothiopyranyl group, such as tetrahydropyran-2-yl, 3-bromotetrahydropyran-2-yl, 4-methoxytetrahydropyran-4-yl, tetrahydrothiopyran-2-yl and 4-methoxytetrahydrothiopyran-4-yl;

a tetrahydrofuranyl or tetrahydrothiofuranyl group, such as tetrahydrofuran-2-yl and tetrahydrothiofuran-2-yl;

a tri(lower alkyl)silyl group such as the trimethylsilyl, triethylsilyl, isopropyldimethylsilyl, tert-butyldimethylsilyl, diisopropylmethylsilyl, di(tert-butyl)methylsilyl and triisopropylsilyl group;

a silyl group substituted with one or two aryl groups and two or one lower alkyl groups, such as diphenylmethylsilyl, diphenylbutylsilyl, diphenylisopropylsilyl, and diisopropylphenylsilyl;

a lower alkoxymethyl group (hereinafter an alkoxy moiety represents a group selected from the group consisting of C₁-C₆ alkoxy group such as methoxy, ethoxy, propoxy, isopropoxy, butoxy, isobutoxy, tert-butoxy, pentyloxy and hexyloxy), such as methoxymethyl, 1,1-dimethyl-1-methoxymethyl, ethoxymethyl, propoxymethyl, isopropoxymethyl, butoxymethyl and tert-butoxymethyl;

a lower alkoxy-lower alkoxylmethyl group, such as the 2-methoxyethoxymethyl group;

a halogeno-lower-alkoxymethyl group, such as the 2,2,2-trichloroethoxymethyl and bis(2-chloroethoxy)methyl group;

a substituted ethyl group, for example an ethyl group substituted with a lower alkoxy group such as the 1-ethoxyethyl or 1-(isopropoxy)ethyl group, and for example a halogenoethyl group such as the 2,2,2-trichloroethyl group;

an aralkyl group, wherein aryl includes a C₆-C₁₄ aryl group such as phenyl, naphthyl, biphenyl, anthryl and phenanthryl group, for example, a lower alkyl group substituted with 1 to 3 aryl groups such as benzyl, α-naphthyl, β-naphthyl, diphenylmethyl, triphenylmethyl, α-naphthyldiphenylmethyl and 9-anthrylmethyl, and for example a lower alkyl group substituted with 1 to 3 aryl groups, which are substituted with lower alkyl, lower alkoxy, nitro, halogen or cyano group, such as the 4-methylbenzyl, 2,4,6-trimethylbenzyl, 3,4,5-trimethylbenzyl, 4-methoxybenzyl, 4-methoxyphenyldiphenylmethyl, 2-nitrobenzyl, 4-nitrobenzyl, 4-chlorobenzyl, 4-bromobenzyl and 4-cyanobenzyl group;

an alkoxycarbonyl group, including a lower alkoxycarbonyl group such as methoxycarbonyl, ethoxycarbonyl, tert-butoxycarbonyl and isobutoxycarbonyl, and for example a lower alkoxycarbonyl group substituted with halogen or tri(lower alkyl)silyl group such as 2,2,2-trichloroethoxycarbonyl and 2-trimethylsilylethoxycarbonyl;

an alkenyloxycarbonyl group, wherein the alkenyl moiety is a C₂-C₆ alkenyl group, such as the vinyloxycarbonyl and allyloxycarbonyl group; and

an aralkyloxycarbonyl group in which the aryl ring is optionally substituted with one or two lower alkoxy or nitro groups, such as the benzyloxycarbonyl, 4-methoxybenzyloxycarbonyl, 3,4-dimethoxybenzyloxycarbonyl, 2-nitrobenzyloxycarbonyl and 4-nitrobenzyloxycarbonyl group.

In one embodiment, the protecting group is the tetrahydropyranyl, tetrahydrothiopyranyl, silyl, aralkyl or aralkyloxycarbonyl group.

In another embodiment, the protecting group is tetrahydropyran-2-yl, 4-methoxytetrahydropyran-4-yl, tetrahydrothiopyran-2-yl, trimethylsilyl, triethylsilyl, tert-butyldimethylsilyl, di(tert-butyl)methylsilyl, diphenylmethylsilyl, benzyl, diphenylmethyl, triphenylmethyl, 4-methylbenzyl, 4-methoxybenzyl, 2-nitrobenzyl, 4-nitrobenzyl, 4-chlorobenzyl, benzyloxycarbonyl, 4-methoxybenzyloxycarbonyl, 2-nitrobenzyloxycarbonyl or 4-nitrobenzyloxycarbonyl group.

In still another embodiment, the protecting group is the trimethylsilyl, tert-butyldimethylsilyl, triphenylmethyl, benzyl or 4-methoxybenzyl group.

A hydroxy protecting group which can be removed by biological method such as hydrolysis in vivo includes a 1-aliphatic acyloxy-lower alkyl group, wherein acyl comprises a C₁-C₁₀ straight or branched chain alkanoyl group, such as formyloxymethyl, acetoxymethyl, dimethylaminoacetoxymethyl, propionyloxymethyl, butyryloxymethyl, pivaloyloxymethyl, valeryloxymethyl, isovaleryloxymethyl, hexanoyloxymethyl, 1-formyloxyethyl, 1-acetoxyethyl, 1-propionyloxyethyl, 1-butyryloxyethyl, 1-pivaloyloxyethyl, 1-valeryloxyethyl, 1-isovaleryloxyethyl, 1-hexanoyloxyethyl, 1-formyloxypropyl, 1-acetoxypropyl, 1-propionyloxypropyl, 1-butyryloxypropyl, 1-pivaloyloxypropyl, 1-valeryloxypropyl, 1-isovaleryloxypropyl, 1-hexanoyloxypropyl, 1-acetoxybutyl, 1-propionyloxybutyl, 1-butyryloxybutyl, 1-pivaloyloxybutyl, 1-acetoxypentyl, 1-propionyloxypentyl, 1-butyryloxypentyl, 1-pivaloyloxypentyl and 1-pivaloyloxyhexyl;

a 1-(aliphatic-acylthio)-(lower alkyl) group, such as formylthiomethyl, acetylthiomethyl, dimethylaminoacetylthiomethyl, propionylthiomethyl, butyrylthiomethyl, pivaloylthiomethyl, valerylthiomethyl, isovalerylthiomethyl, hexanoylthiomethyl, 1-formylthioethyl, 1-acetylthioethyl, 1-propionylthioethyl, 1-butyrylthioethyl, 1-pivaloylthioethyl, 1-valerylthioethyl, 1-isovalerylthioethyl, 1-hexanoylthioethyl, 1-formylthiopropyl, 1-acetylthiopropyl, 1-propionylthiopropyl, 1-butyrylthiopropyl, 1-pivaloylthiopropyl, 1-valerylthiopropyl, 1-isovalerylthiopropyl, 1-hexanoylthiopropyl, 1-acetylthiobutyl, 1-propionylthiobutyl, 1-butyrylthiobutyl, 1-pivaloylthiobutyl, 1-acetylthiopentyl, 1-propionylthiopentyl, 1-butyrylthiopentyl, 1-pivaloylthiopentyl and 1-pivaloylthiohexyl;

a 1-(cycloalkylcarbonyloxy)-(lower alkyl) group, such as cyclopentylcarbonyloxymethyl, cyclohexylcarbonyloxymethyl, 1-cyclopentylcarbonyloxyethyl, 1-cyclohexylcarbonyloxyethyl, 1-cyclopentylcarbonyloxypropyl, 1-cyclohexylcarbonyloxypropyl, 1-cyclopentylcarbonyloxybutyl and 1-cyclohexylcarbonyloxybutyl;

a 1-aromatic acyloxy)-(lower alkyl) group, wherein the aromatic acyl moiety comprises a C₆-C₁₀ arylcarbonyl groups such as the benzoyloxymethyl group;

a 1-(lower alkoxycarbonyloxy)-(lower alkyl) group, including methoxycarbonyloxymethyl, ethoxycarbonyloxymethyl, propoxycarbonyloxymethyl, isopropoxycarbonyloxymethyl, butoxycarbonyloxymethyl, isobutoxycarbonyloxymethyl, pentyloxycarbonyloxymethyl, hexyloxycarbonyloxymethyl, 1-(methoxycarbonyloxy)ethyl, 1-(ethoxycarbonyloxy)ethyl, 1-(propoxycarbonyloxy)ethyl, 1-(isopropoxycarbonyloxy)ethyl, 1-(butoxycarbonyloxy)ethyl, 1-(isobutoxycarbonyloxy)ethyl, 1-(tert-butoxycarbonyloxy)ethyl, 1-(pentyloxycarbonyloxy)ethyl, 1-(hexyloxycarbonyloxy)ethyl, 1-(methoxycarbonyloxy)propyl, 1-(ethoxycarbonyloxy)propyl, 1-(propoxycarbonyloxy)propyl, 1-(isopropoxycarbonyloxy)propyl, 1-(butoxycarbonyloxy)propyl, 1-(isobutoxycarbonyloxy)propyl, 1-(pentyloxycarbonyloxy)propyl, 1-(hexyloxycarbonyloxy)propyl, 1-(methoxycarbonyloxy)butyl, 1-(ethoxycarbonyloxy)butyl, 1-(propoxycarbonyloxy)butyl, 1-(isopropoxycarbonyloxy)butyl, 1-(butoxycarbonyloxy)butyl, 1-(iosobutoxycarbonyloxy)butyl, 1-(methoxycarbonyloxy)pentyl, 1-(ethoxycarbonyloxy)pentyl, 1-(methoxycarbonyloxy)hexyl and 1-(ethoxycarbonyloxy)hexyl;

a 1-(cycloalkyloxycarbonyloxy)-(lower alkyl) group, including cyclopentyloxycarbonyloxymethyl, cyclohexyloxycarbonyloxymethyl, 1-(cyclopentyloxycarbonyloxy)ethyl, 1-(cyclohexyloxycarbonyloxy)ethyl, 1-(cyclopentyloxycarbonyloxy)propyl, 1-(cyclohexyloxycarbonyloxy)propyl, 1-(cyclopentyloxycarbonyloxy)butyl, 1-(cyclohexyloxycarbonyloxy)butyl, 1-(cyclopentyloxycarbonyloxy)pentyl, (cyclopentyloxycarbonyloxy)pentyl, 1-(cyclohexyloxycarbonyloxy)pentyl, 1-(cyclopentyloxycarbonyloxy)hexyl and 1-(cyclohexyloxycarbonyloxy)hexyl;

a phthalidyl group, such as phthalidyl, dimethylphthalidyl and dimethoxyphthalidyl;

an oxodioxolenylmethyl group, such as (5-phenyl-2-oxo-1,3-dioxolen-4-yl)methyl, [5-(4-methylphenyl)-2-oxo-1,3-dioxolen-4-yl]methyl, [5-(4-methoxyphenyl)-2-oxo-1,3-dioxolen-4-yl]methyl, [5-(4-fluorophenyl)-2-oxo-1,3-dioxolen-4-yl]methyl, [5-(4-chlorophenyl)-2-oxo-1,3-dioxolen-4-yl]methyl, (2-oxo-1,3-dioxolen-4-yl)methyl, (5-methyl-2-oxo-1,3-dioxolen-4-yl)methyl, (5-ethyl-2-oxo-1,3-dioxolen4-yl)methyl, (5-propyl-2-oxo-1,3-dioxolen-4-yl)methyl, (5-isopropyl-2-oxo-1,3-dioxolen-4-yl)methyl and (5-butyl-2-oxo-1,3-dioxolen-4-yl)methyl;

a carbamoyl group, including a carbamoyl group substituted with one or two lower alkyl groups;

a lower alkyl-dithioethyl group, such as methyldithioethyl, ethyldithioethyl, propyldithioethyl, butyldithioethyl, pentyldithioethyl and hexyldithioethyl group; and

a 1-(acyloxy)alkyloxycarbonyl group, such as the pivaloyloxymethyloxycarbonyl group and the like.

In another embodiment, the hydroxy protecting group which can be removed by biological method such as hydrolysis in vivo is a 1-(aliphatic acyloxy)-(lower alkyl) group, a 1-(cycloalkylcarbonyloxy)-(lower alkyl) group, a 1-(lower alkoxycarbonyloxy)-(lower alkyl) group, a 1-(cycloalkyloxycarbonyloxy)-(lower alkyl) group, a phthalidyl or an oxodioxolenylmethyl group.

In still another embodiment, the hydroxy protecting group which can be removed by biological method such as hydrolysis in vivo is an acetoxymethyl, propionyloxymethyl, butyryloxymethyl, pivaloyloxymethyl, valeryloxymethyl, 1-acetoxyethyl, butyryloxyethyl, 1-pivaloyloxyethyl, cyclopentylcarbonyloxymethyl, cyclohexylcarbonyloxymethyl, 1-cyclopentylcarbonyloxyethyl, 1-cyclohexylcarbonyloxyethyl, methoxycarbonyloxymethyl, ethoxycarbonyloxymethyl, propoxycarbonyloxymethyl, isopropoxycarbonyloxymethyl, butoxycarbonyloxymethyl, isobutoxycarbonyloxymethyl, 1-(methoxycarbonyloxy)ethyl, 1-(ethoxycarbonyloxy)ethyl, 1-(isopropoxycarbonyloxy)ethyl, cyclopentyloxycarbonyloxymethyl, cyclohexyloxycarbonyloxymethyl, 1-(cyclopentyloxycarbonyloxy)ethyl, 1-(cyclohexyloxycarbonyloxy)ethyl, phthalidyl, (5-phenyl-2-oxo-1,3-dioxolen-4-yl)methyl, [5-(4-methylphenyl)-2-oxo-1,3-dioxolen-4-yl]methyl, (5-methyl-2-oxo-1,3-dioxolen-4-yl)methyl or (5-ethyl-2-oxo-1,3-dioxolen-4-yl)methyl group.

In another embodiment, the hydroxy protecting group which can be removed by biological method such as hydrolysis in vivo is acetoxymethyl, propionyloxymethyl, butyryloxymethyl, pivaloyloxymethyl, valeryloxymethyl, cyclopentylcarbonyloxymethyl, cyclohexylcarbonyloxymethyl, methoxycarbonyloxymethyl, ethoxycarbonyloxymethyl, propoxycarbonyloxymethyl, isopropoxycarbonyloxymethyl, butoxycarbonyloxymethyl, isobutoxycarbonyloxymethyl, cyclopentyloxycarbonyloxymethyl, cyclohexyloxycarbonyloxymethyl, (5-phenyl-2-oxo-1,3-dioxolen-4-yl)methyl. [5-(4-methylphenyl)-2-oxo-1,3-dioxolen-4-yl]methyl, (5-methyl-2-oxo-1,3-dioxolen-4-yl)methyl or (5-ethyl-2-oxo-1,3-dioxolen-4-yl)methyl.

The term “pharmaceutically acceptable ester, ether and N-alkylcarbamoyl derivatives” refers to a derivative that is a useful medicament without significant toxicity.

The ester residue of ester derivatives includes carbonyl and oxycarbonyl groups to which a straight or branched chain C₁-C₂₁ alkyl group is attached, including methyl, ethyl, propyl, isopropyl, butyl, isobutyl, sec-butyl, tert-butyl, pentyl, isopentyl, 2-methylbutyl, neopentyl, 1-ethylpropyl, hexyl, isohexyl, 4-methylpentyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, heptyl, 1-methylhexyl, 2-methylhexyl, 3-methylhexyl, 4-methylhexyl, 5-methylhexyl, 1-propylbutyl, 4,4-dimethylpentyl, octyl, 1-methylheptyl, 2-methylheptyl, 3-methylheptyl, 4-methylheptyl, 5-methylheptyl, 6-methylheptyl, 1-propylpentyl, 2-ethylhexyl, 5,5-dimethylhexyl, nonyl, 3-methyloctyl, 4-methyloctyl, 5-methyloctyl, 6-methyloctyl, 1-propylhexyl, 2-ethylheptyl. 6,6-dimethylheptyl, decyl, 1-methylnonyl, 3-methylnonyl, 8-methylnonyl, 3-ethyloctyl, 3,7-dimethyloctyl, 7,7-dimethyloctyl, undecyl, 4,8-dimethylnonyl, dodecyl, tridecyl, tetradecyl, pentadecyl, 3,7,11-trimethyldodecyl, hexadecyl, 4,8,12-trimethyltridecyl, 1-methylpentadecyl, 14-methylpentadecyl, 13,13-dimethyltetradecyl, heptadecyl, 15-methylhexadecyl, octadecyl, 1-methylheptadecyl, nonadecyl, icosyl, 3,7,11,15-tetramethylhexadecyl and henicosyl groups;

a carbonyl and oxycarbonyl group to which a straight or branched chain C₂-C₂₁ alkenyl or alkynyl group is attached, wherein said alkenyl or alkynyl group includes ethenyl, 1-propenyl, 2-propenyl, 1-methyl-2-propenyl, 1-methyl-1-propenyl, 2-methyl-1-propenyl, 2-methyl-2-propenyl, 2-ethyl-2-propenyl, 1-butenyl, 2-butenyl, 1-methyl-2-butenyl, 1-methyl-1-butenyl, 3-methyl-2-butenyl, 1-ethyl-2-butenyl, 3-butenyl, 1-methyl-3-butenyl, 2-methyl-3-butenyl, 1-ethyl-3-butenyl, 1-pentenyl, 2-pentenyl, 1-methyl-2-pentenyl, 2-methyl-2-pentenyl, 3-pentenyl, 1-methyl-3-pentenyl, 2-methyl-3-pentenyl, 4-pentenyl, 1-methyl-4-pentenyl, 2-methyl-4-pentenyl, 1-hexenyl, 2-hexenyl, 3-hexenyl, 4-hexenyl, 5-hexenyl, cis-8-heptadecenyl, cis, cis-8,11-heptadecadienyl, cis, cis, cis-8,11,14-heptadecatrienyl, cis-10-nonadecenyl, and cis-12-icosenyl;

a carbonyl or oxycarbonyl group to which a straight or branched chain C₂-C₂₁ alkenyl or alkynyl group is attached, including ethynyl, 2-propynyl, 1-methyl-2-propynyl, 2-methyl-2-propynyl, 2-ethyl-2-propynyl, 2-butynyl, 1-methyl-2-butynyl, 2-methyl-2-butynyl, 1-ethyl-2-butynyl, 3-butynyl, 1-methyl-3-butynyl, 2-methyl-3-butynyl, 1-ethyl-3-butynyl, 2-pentynyl, 1-methyl-2-pentynyl, 2-methyl-2-pentynyl, 3-pentynyl, 1-methyl-3-pentynyl, 2-methyl-3-pentynyl, 4-pentynyl, 1-methyl-4-pentynyl, 2-methyl-4-pentynyl, 2-hexynyl, 3-hexynyl, 4-hexynyl and 5-hexynyl;

a carbonyl and oxycarbonyl group to which straight or branched chain C₁-C₂₁, alkyl group which is optionally substituted by one or more lower alkoxy groups, halogen (hereinafter for example fluorine, chlorine, bromine and iodine, preferably fluorine and chlorine) and nitro, in which said substituted alkyl group includes methoxymethyl, ethoxymethyl, methoxyethyl, ethoxyethyl, trifluoromethyl, trichloromethyl, difluoromethyl, dichloromethyl, dibromomethyl, fluoromethyl, 2,2,2-trifluoroethyl, 2,2,2-trichloroethyl, 2-bromoethyl, 2-chloroethyl, 2-fluoroethyl, 2-iodoethyl, 3-chloropropyl, 4-fluorobutyl, 6-iodohexyl, 2,2-dibromoethyl, nitromethyl, dinitromethyl, 1-nitroethyl, 2-nitroethyl and 1,2-dinitroethyl;

a carbonyl and oxycarbonyl group to which a (C₆-C₁₀ aryl)-(C₁-C₂₁)alkyl group wherein said aryl moiety is optionally substituted by one or more lower alkyl groups, lower alkoxy groups, halo or nitro groups, in which said arylalkyl group includes benzyl, α-naphthylmethyl, β-naphthylmethyl, indenylmethyl, phenanthrenylmethyl, anthracenylmethyl, diphenylmethyl, triphenylmethyl, 1-phenethyl, 2-phenethyl, 1-naphthylethyl, 2-naphthylethyl, 1-phenylpropyl, 2-phenylpropyl, 3-phenylpropyl, 1-naphthylpropyl, 2-naphthylpropyl, 3-naphthylpropyl, 1-phenylbutyl, 2-phenylbutyl, 3-phenylbutyl, 4-phenylbutyl, 1-naphthylbutyl, 2-naphthylbutyl, 3-naphthylbutyl, 4-naphthylbutyl, 1-phenylpentyl, 2-phenylpentyl, 3-phenylpentyl, 4-phenylpentyl, 5-phenylpentyl, 1-naphthylpentyl, 2-naphthylpentyl, 3-naphthylpentyl, 4-naphthylpentyl, 5-naphthylpentyl, 1-phenylhexyl, 2-phenylhexyl, 3-phenylhexyl, 4-phenylhexyl, 5-phenylhexyl, 6-phenylhexyl, 1-naphthylhexyl, 2-naphthylhexyl, 3-naphthylhexyl, 4-naphthylhexyl, 5-naphthylhexyl and 6-naphthylhexyl;

a carbonyl and oxycarbonyl group to which a C₆-C₁₀ aryl group which is optionally substituted by one or more lower alkyl groups, lower alkoxy groups, halo and nitro groups, in which said aryl group includes phenyl, naphthyl, 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl, 3,5-difluorophenyl, 2,5-difluorophenyl, 2,6-difluorophenyl, 2,4-difluorophenyl, 3,5-dibromophenyl, 2,5-dibromophenyl 2,6-dichlorophenyl, 2,4-dichlorophenyl, 2,3,6-trifluorophenyl, 2,3,4-trifluorophenyl, 3,4,5-trifluorophenyl, 2,5,6-trifluorophenyl, 2,4,6-trifluorophenyl, 2,3,6-tribromophenyl, 2,3,4-tribromophenyl, 3,4,5-tribromophenyl, 2,5,6-trichlorophenyl, 2,4,6-trichlorophenyl, 1-fluoro-2-naphthyl, 2-fluoro-1-naphthyl, 3-fluoro-1-naphthyl, 1-chloro-2-naphthyl, 2-chloro-1-naphthyl, 3-bromo-1-naphthyl, 3,8-difluoro-1-naphthyl, 2,3-difluoro-1-naphthyl, 4,8-difluoro-1-naphthyl, 5,6-difluoro-1-naphthyl, 3,8-dichloro-1-naphthyl, 2,3-dichloro-1-naphthyl, 4,8-dibromo-1-naphthyl, 5,6-dibromo-1-naphthyl, 2,3,6-trifluoro-1-naphthyl, 2,3,4-trifluoro-1-naphthyl, 3,4,5-trifluoro-1-naphthyl, 4,5,6-trifluoro-1-naphthyl, 2,4,8-trifluoro-1-naphthyl, 2-methylphenyl, 3-methylphenyl, 4-methylphenyl, 2-ethylphenyl, 3-propylphenyl, 4-ethylphenyl, 2-butylphenyl, 3-pentylphenyl, 4-pentylphenyl, 3,5-dimethylphenyl, 2,5-dimethylphenyl, 2,6-dimethylphenyl, 2,4-dimethylphenyl, 3,5-dibutylphenyl, 2,5-dipentylphenyl, 2,6-dipropylmethylphenyl, 2,4-dipropylphenyl, 2,3,6-trimethylphenyl, 2,3,4-trimethylphenyl, 3,4,5-trimethylphenyl, 2,5,6-trimethylphenyl, 2,4,6-trimethylphenyl, 2,3,6-tributylphenyl, 2,3,4-tripentylphenyl, 3,4,5-tributylphenyl, 2,5,6-tripropylmethylphenyl, 2,4,6-tripropylphenyl, 1-methyl-2-naphthyl, 2-methyl-1-naphthyl, 3-methyl-1-naphthyl, 1-ethyl-2-naphthyl, 2-propyl-1-naphthyl, 3-butyl-1-naphthyl, 3,8-dimethyl-1-naphthyl, 2,3-dimethyl-1-naphthyl, 4,8-dimethyl-1-naphthyl, 5,6-dimethyl-1-naphthyl, 3,8-diethyl-1-naphthyl, 2,3-dipropyl-1-naphthyl, 4,8-dipentyl-1-naphthyl, 5,6-dibutyl-1-naphthyl, 2,3,6-trimethyl-1-naphthyl, 2,3,4-trimethyl-1-naphthyl, 3,4,5-trimethyl-1-naphthyl, 4,5,6-trimethyl-1-naphthyl, 2,4,8-trimethyl-1-naphthyl, 2-methoxyphenyl, 3-methoxyphenyl, 4-methoxyphenyl, 2-ethoxyphenyl, 3-propoxphenyl, 4-ethoxyphenyl, 2-butoxyphenyl, 3-pentyloxyphenyl, 4-pentyloxyphenyl, 3,5-dimethoxyphenyl, 2,5-dimethoxyphenyl, 2,6-dimethoxyphenyl, 2,4-dimethoxyphenyl, 3,5-dibutoxyphenyl, 2,5-dipentyloxyphenyl, 2,6-dipropoxymethoxphenyl, 2,4-dipropoxyphenyl, 2,3,6-trimethoxyphenyl, 2,3,4-trimethoxyphenyl, 3,4,5-trimethoxyphenyl, 5,6-trimethoxyphenyl, 2,4,6-trimethoxyphenyl, 2,3,6-tributoxyphenyl, 2,3,4-tripentyloxyphenyl, 3,4,5-tributoxyphenyl, 2,5,6-tripropoxyphenyl, 2,4,6-tripropoxyphenyl, 1-methoxy-2-naphthyl, 2-methoxy-1-naphthyl, 3-methoxy-1-naphthyl, 1-ethoxy-2-naphthyl, 2-propoxy-1-naphthyl, 3-butoxy-1-naphthyl, 3,8-dimethoxy-1-naphthyl, 2,3-dimethoxy-1-naphthyl, 4,8-dimethoxy-1-naphthyl 5,6-dimethoxy-1-naphthyl, 3,8-diethoxy-1-naphthyl, 2,3-dipropoxy-1-naphthyl, 4,8-dipentyloxy-1-naphthyl, 5,6-dibutoxy-1-naphthyl, 2,3,6-trimethoxy-1-naphthyl, 2,3,4-trimethoxy-1-naphthyl, 3,4,5-trimethoxy-1-naphthyl, 4,5,6-trimethoxy-1-naphthyl, 2,4,8-trimethoxy-1-naphthyl, 2-nitrophenyl, 3-nitrophenyl, 4-nitrophenyl, 3,5-dinitrophenyl, 2,5-dinitrophenyl 2,6-dinitrophenyl, 2,4-dinitrophenyl, 2,3,6-trinitrophenyl, 2,3,4-trinitrophenyl, 3,4,5-trinitrophenyl, 2,5,6-trinitrophenyl, 2,4,6-trinitrophenyl, 1-nitro-2-naphthyl, 2-nitro-1-naphthyl, 3-nitro-1-naphthyl, 3,8-dinitro-1-naphthyl, 2,3-dinitro-1-naphthyl, 4,8-dinitro-1-naphthyl, 5,6-dinitro-1-naphthyl. 2,3,6-trinitro-1-naphthyl, 2,3,4-trinitro-1-naphthyl, 3,4,5-trinitro-1-naphthyl, 4,5,6-trinitro-1-naphthyl and 2,4,8-trinitro-1-naphthyl;

a carboxy(C₁-C₁₀)alkylcarbonyl group such as succinoyl, glutaroyl, and adipoyl;

a residue of salt of a phosphate diester which independently has two lower alkyl groups; and

a residue an amino acid which is optionally protected with a tert-butyloxycarbonyl, benzyloxycarbonyl or trityl group, including but not limited to, glycine, alanine, valine, leucine, isoleucine, phenylalanine, proline, tryptophan, glutamine, glutamic acid, isonipecotic acid, aminobenzoic acid, 4-aminophenylacetic acid, anthranilic acid, 3-amino-2-naphthoic acid, nicotinic acid, isonicotinic acid, aminooctanoic acid, aminononanoic acid, aminopentanoic acid, aminoundecanoic acid, and 15-(amino)-4,7,10,13-tetraoxapentadecanoic acid (topdec).

A preferable ester residue of ester derivatives is R⁶ CO— or R⁶ OCO— group wherein R⁶ is selected from the group consisting of hydrogen; a C₁-C₂₁ alkyl group; a C₂-C₂₁ alkenyl or alkynyl group having 1 to 3 double or triple bonds; a C₁-C₂₁ alkyl group substituted with 1 to 4 substituents selected from the group consisting of lower alkoxy, halo and nitro groups; a C₁-C₂₁ alkyl group substituted with 1 to 3 C₆-C₁₀ aryl groups which are optionally substituted with 1 to 4 substituents selected from the group consisting of lower alkyl, lower alkoxy, halo and nitro groups; and a C₆-C₁₀ aryl group which is optionally substituted with 1 to 4 substituents selected from the group consisting of lower alkyl, lower alkoxy, halo, and nitro groups.

A more preferable ester residue of ester derivatives is R⁶ CO— or R⁶ OCO— group wherein R⁶ is selected from the group consisting of hydrogen; a C₁-C₂₁ alkyl group; a C₂-C₂₁ alkenyl group having 1 to 3 double bonds; a C₂-C₆ alkynyl group having one triple bond; a C₁-C₆ alkyl group substituted with 1 to 4 substituents selected from the group consisting of C₁-C₄ alkoxy, halo and nitro groups; a C₁-C₆ alkyl group substituted with 1 to 3 C₆-C₁₀ aryl groups which are optionally substituted with 1 to 3 substituents selected from the group consisting of C₁-C₄ alkyl, C₁-C₄ alkoxy, halo and nitro groups; and a C₆-C₁₀ aryl group which is optionally substituted with 1 to 3 substituents selected from the group consisting of C₁-C₄ alkyl, C₁-C₄ alkoxy, halo and nitro groups.

A more preferable ester residue of ester derivatives is R⁶ CO— or R⁶ OCO— group wherein R⁶ is selected from the group consisting of a C₁-C₂₁ alkyl group; a C₆-C₂₀ alkenyl group having 1 to 3 double bonds; a C₂-C₆ alkynyl group having one triple bond; a C₁-C₆ alkyl group substituted with one substituent selected from the group consisting of C₁-C₄ alkoxy and nitro groups; a C₁-C₆ alkyl group substituted with 1 to 3 substituents selected from the group consisting of halogen; a C₁-C₄ alkyl group substituted with 1 to 3 phenyl or naphthyl groups which are optionally substituted with 1 to 3 substituents selected from the group consisting of C₁-C₄alkyl, C₁-C₄ alkoxy, halo and nitro groups; and a phenyl or naphthyl group which is optionally substituted with 1 to 3 substituents selected from the group consisting of C₁-C₄ alkyl, C₁-C₄ alkoxy, halo and nitro groups.

A more preferable ester residue of ester derivatives is R⁶ CO— or R⁶ OCO— group wherein R⁶ is selected from the group consisting of a C₆-C₂₀ alkyl group; a C₁₀-C₂₀ alkenyl group having 1 to 3 double bonds; a C₃-C₅ alkynyl group having one triple bond; a C₁-C₄ alkyl group substituted with one substituent selected from the group consisting of C₁-C₄ alkoxy and nitro groups: a C₁-C₄ alkyl group substituted with 1 to 3 substituents selected from the group consisting of fluoro and chloro groups: a C₁-C₄ alkyl group substituted with 1 to 3 phenyl groups which are optionally substituted with 1 or 2 substituents selected from the group consisting of C₁-C₂ alkyl, C₁-C₄ alkoxy, fluoro and chloro groups; and a phenyl group which is optionally substituted with 1 to 3 substituents selected from the group consisting of C₁-C₂ alkyl, C₁-C₄ alkoxy, fluoro and chloro groups.

A more preferable ester residue of ester derivatives is R⁶ CO— or R⁶ OCO— group wherein R⁶ is selected from the group consisting of a C₆-C₂₀ alkyl group, a C₁₀-C₂₀ alkenyl group having 1 to 3 double bonds, a C₃-C₅ alkynyl group having one triple bond; a C₁-C₄ alkyl group substituted with one substituent selected from the group consisting of C₁-C₄ alkoxy, fluoro, chloro and nitro groups; a C₁-C₄ alkyl group substituted with 1 to 3 phenyl groups which are optionally substituted with 1 or 2 substituents selected from the group consisting of C₁-C₂ alkyl, C₁-C₄ alkoxy, fluoro and chloro groups; and a phenyl group which is optionally substituted with 1 to 3 substituents selected from the group consisting of C₁-C₂ alkyl C₁-C₄ alkoxy, fluoro and chloro groups.

A still more preferable ester residue of ester derivatives is R⁶ CO— or R⁶ OCO— group wherein R⁶ is selected from the group consisting of a C₆-C₂₀ alkyl group; a C₁₀-C₂₀ alkenyl group having 1 to 3 double bonds; a C₃-C₅ alkynyl group having one triple bond; a C₁-C₄ alkyl group substituted with one substituent selected from the group consisting of C₁-C₄ alkoxy groups, and a C₁-C₄alkyl group substituted with 1 or 2 phenyl groups which are optionally substituted with 1 or 2 substituents selected from the group consisting of C₁-C₂ alkyl, C₁-C₄ alkoxy, fluoro and chloro groups.

A most preferable ester residue of ester derivatives is R⁶ CO— or R⁶ OCO— group wherein R⁶ is selected from the group consisting of a C₆-C₂₀ alkyl group and a C₁₀-C₂₀ alkenyl group having 1 to 3 double bonds.

An ether residue of ether derivatives is selected from the group consisting of straight or branched chain C₁-C₂₁ alkyl group, such as the methyl, ethyl, propyl, isopropyl, butyl, isobutyl, sec-butyl, tert-butyl, pentyl, isopentyl, 2-methylbutyl, neopentyl, 1-ethylpropyl, hexyl, isohexyl, 4-methylpentyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, heptyl, 1-methylhexyl, 2-methylhexyl, 3-methylhexyl, 4-methylhexyl, 5-methylhexyl, 1-propylbutyl, 4,4-dimethylpentyl, octyl, 1-methylheptyl, 2-methylheptyl, 3-methylheptyl, 4-methylheptyl, 5-methylheptyl, 6-methylheptyl, 1-propylpentyl, 2-ethylhexyl, 5,5-dimethylhexyl, nonyl, 3-methyloctyl, 4-methyloctyl, 5-methyloctyl, 6-methyloctyl, 1-propylhexyl, 2-ethylheptyl, 6,6-dimethylheptyl, decyl, 1-methylnonyl, 3-methylnonyl, 8-methylnonyl, 3-ethyloctyl, 3,7-dimethyloctyl, 7,7-dimethyloctyl, undecyl, 4,8-dimethylnonyl, dodecyl, tridecyl, tetradecyl, pentadecyl, 3,7,11-trimethyldodecyl, hexadecyl, 4,8,12-trimethyltridecyl, 1-methylpentadecyl, 14-methylpentadecyl, 13,13-dimethyltetradecyl, heptadecyl, 15-methylhexadecyl, octadecyl, 1-methylheptadecyl, nonadecyl, icosyl, 3,7,11,15-tetramethylhexadecyl and henicosyl groups;

straight or branched chain C₂-C₂₁ alkenyl or alkynyl group, such as ethenyl, 1-propenyl, 2-propenyl, 1-methyl-2-propenyl, 1-methyl-1-propenyl, 2-methyl-1-propenyl, 2-methyl-2-propenyl, 2-ethyl-2-propenyl, 1-butenyl, 2-butenyl, 1-methyl-2-butenyl, 1-methyl-1-butenyl, 3-methyl-2-butenyl, 1-ethyl-2-butenyl, 3-butenyl, 1-methyl-3-butenyl, 2-methyl butenyl, 1-ethyl-3-butenyl, 1-pentenyl, 2-pentenyl, 1-methyl-2-pentenyl, 2-methyl-2-pentenyl, 3-pentenyl, 1-methyl-3-pentenyl, 2-methyl-3-pentenyl, 4-pentenyl, 1-methyl-4-pentenyl, 2-methyl-4-pentenyl, 1-hexenyl, 2-hexenyl, 3-hexenyl, 4-hexenyl, 5-hexenyl, cis-8-heptadecenyl, cis, cis-8,11-heptadecadienyl, cis, cis, cis-8,11,14-heptadecatrienyl, cis-10-nonadecenyl, cis-12-icosenyl, ethynyl, 2-propynyl, 1-methyl-2-propynyl, 2-methyl-2-propynyl, 2-ethyl-2-propynyl, 2-butynyl, 1-methyl-2-butynyl, 2-methyl-2-butynyl, 1-ethyl-2-butynyl, 3-butynyl, 1-methyl-3-butynyl, 2-methyl-3-butynyl, 1-ethyl-3-butynyl, 2-pentynyl, 1-methyl-2-pentynyl, 2-methyl-2-pentynyl, 3-pentynyl, 1-methyl-3-pentynyl, 2-methyl-3-pentynyl, 4-pentynyl, 1-methyl-4-pentynyl, 2-methyl-4-pentynyl, 2-hexynyl, 3-hexynyl, 4-hexynyl and 5-hexynyl;

straight or branched chain C₁-C₂₁ alkyl group which has one or more substituents selected from the group consisting of lower alkoxy, halogen (hereinafter for example fluorine, chlorine, bromine and iodine, preferably fluorine and chlorine) and nitro groups, such as methoxymethyl, ethoxymethyl, methoxyethyl, ethoxyethyl, trifluoromethyl, tnchloromethyl, difluoromethyl, dichloromethyl, dibromomethyl, fluoromethyl, 2,2,2-trifluoroethyl, 2,2,2-trichloroethyl, 2-bromoethyl, 2-chloroethyl, 2-fluoroethyl, 2-iodoethyl, 3-chloropropyl, 4-fluorobutyl, 6-iodohexyl, 2,2-dibromoethyl, nitromethyl, dinitromethyl, 1-nitroethyl, 2-nitroethyl and 1,2-dinitroethyl;

(C₆-C₁₀)aryl-(C₁-C₂₁)alkyl group wherein said aryl moiety optionally has one or more substituents selected from the group consisting of lower alkyl, lower alkoxy, halo and nitro group, such as benzyl, α-naphthylmethyl, β-naphthylmethyl, indenylmethyl, phenanthrenylmethyl, anthracenylmethyl, diphenylmethyl, triphenylmethyl, 1-phenethyl, 2-phenethyl, 1-naphthylethyl, 2-naphthylethyl, 1-phenylpropyl, 2-phenylpropyl, 3-phenylpropyl, 1-naphthylpropyl, 2-naphthylpropyl, 3-naphthylpropyl, 1-phenylbutyl, 2-phenylbutyl, 3-phenylbutyl, 4-phenylbutyl, 1-naphthylbutyl, 2-naphthylbutyl, 3-naphthylbutyl, 4-naphthylbutyl. 1-phenylpentyl, 2-phenylpentyl, 3-phenylpentyl, 4-phenylpentyl, 5-phenylpentyl, 1-naphthylpentyl, 2-naphthylpentyl, 3-naphthylpentyl, 4-naphthylpentyl, 5-naphthylpentyl, 1-phenylhexyl, 2-phenylhexyl, 3-phenylhexyl, 4-phenylhexyl, 5-phenylhexyl, 6-phenylhexyl, 1-naphthylhexyl, 2-naphthylhexyl, 3-naphthylhexyl, 4-naphthylhexyl, 5-naphthylhexyl and 6-naphthylhexyl; and

C₆-C₁₀ aryl group which optionally has one or more substituents selected from the group consisting of lower alkyl, lower alkoxy, halo and nitro groups, such as phenyl, naphthyl, 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl, 3,5-difluorophenyl, 2,5-difluorophenyl, 2,6-difluorophenyl, 2,4-difluorophenyl, 3,5-dibromophenyl, 2,5-dibromophenyl, 2,6-dichlorophenyl, 2,4-dichlorophenyl, 2,3,6-trifluorophenyl, 2,3,4-trifluorophenyl, 3,4,5-trifluorophenyl, 2,5,6-trifluorophenyl, 2,4,6-trifluorophenyl, 2,3,6-tribromophenyl, 2,3,4-tribromophenyl, 3,4,5-tribromophenyl, 2,5,6-trichlorophenyl, 2,4,6-trichlorophenyl, 1-fluoro-2-naphthyl, 2-fluoro-1-naphthyl, 3-fluoro-1-naphthyl, 1-chloro-2-naphthyl, 2-chloro-1-naphthyl, 3-bromo-1-naphthyl, 3,8-difluoro-1-naphthyl, 2,3-difluoro-1-naphthyl, 4,8-difluoro-1-naphthyl, 5,6-difluoro-1-naphthyl, 3,8-dichloro-1-naphthyl, 2,3-dichloro-1-naphthyl, 4,8-dibromo-1-naphthyl, 5,6-dibromo-1-naphthyl, 2,3,6-trifluoro-1-naphthyl, 2,3,4-trifluoro-1-naphthyl, 3,4,5-trifluoro-1-naphthyl, 4,5,6-trifluoro-1-naphthyl, 2,4,8-trifluoro-1-naphthyl, 2-methylphenyl, 3-methylphenyl, 4-methylphenyl, 2-ethylphenyl, 3-propylphenyl, 4-ethylphenyl, 2-butylphenyl, 3-pentylphenyl, 4-pentylphenyl, 3,5-dimethylphenyl, 2,5-dimethylphenyl, 2,6-dimethylphenyl, 2,4-dimethylphenyl, 3,5-dibutylphenyl, 2,5-dipentylphenyl, 2,6-dipropylmethylphenyl, 2,4-dipropylphenyl, 2,3,6-trimethylphenyl, 2,3,4-trimethylphenyl, 3,4,5-trimethylphenyl, 2,5,6-trimethylphenyl, 2,4,6-trimethylphenyl, 2,3,6-tributylphenyl, 2,3,4-tripentylphenyl, 3,4,5-tributylphenyl, 2,5,6-tripropylmethylphenyl, 2,4,6-tripropylphenyl, 1-methyl-2-naphthyl, 2-methyl-1-naphthyl, 3-methyl-1-naphthyl, 1-ethyl-2-naphthyl, 2-propyl-1-naphthyl, 3-butyl-1-naphthyl, 3,8-dimethyl-1-naphthyl, 2,3-dimethyl-1-naphthyl, 4,8-dimethyl-1-naphthyl, 5,6-dimethyl-1-naphthyl, 3,8-diethyl-1-naphthyl, 2,3-dipropyl-1-naphthyl, 4,8-dipentyl-1-naphthyl, 5,6-dibutyl-1-naphthyl, 2,3,6-trimethyl-1-naphthyl, 2,3,4-trimethyl-1-naphthyl, 3,4,5-trimethyl-1-naphthyl, 4,5,6-trimethyl-1-naphthyl, 2,4,8-trimethyl-1-naphthyl, 2-methoxyphenyl, 3-methoxyphenyl, 4-methoxyphenyl, 2-ethoxyphenyl, 3-propoxyphenyl, 4-ethoxyphenyl, 2-butoxyphenyl, 3-pentoxyphenyl, 4-pentyloxyphenyl, 3,5-dimethoxyphenyl, 2,5-dimethoxyphenyl, 2,6-dimethoxyphenyl, 2,4-dimethoxyphenyl, 3,5-dibutoxyphenyl, 2,5-dipentyloxyphenyl, 2,6-dipropoxymethoxyphenyl, 2,4-dipropoxyphenyl, 2,3,6-trimethoxyphenyl, 2,3,4-trimethoxyphenyl, 3,4,5-trimethoxyphenyl, 2,5,6-trimethoxyphenyl, 2,4,6-trimethoxyphenyl, 2,3,6-tributoxyphenyl, 2,3,4-tripentyloxyphenyl, 3,4,5-tributoxyphenyl, 2,5,6-tripropoxyphenyl, 2,4,6-tripropoxyphenyl, 1-methoxy-2-naphthyl, 2-methoxy-1-naphthyl, 3-methoxy-1-naphthyl, 1-ethoxy-2-naphthyl, 2-propoxy-1-naphthyl, 3-butoxy-1-naphthyl, 3,8-dimethoxy-1-naphthyl, 2,3-dimethoxy-1-naphthyl, 4,8-dimethoxy-1-naphthyl, 5,6-dimethoxy-1-naphthyl, 3,8-diethoxy-1-naphthyl, 2,3-dipropoxy-1-naphthyl, 4,8-dipentyloxy-1-naphthyl, 5,6-dibutoxy-1-naphthyls 2,3,6-trimethoxy-1-naphthyl, 2,3,4-trimethoxy-1-naphthyl, 3,4,5-trimethoxy-1-naphthyl, 4,5,6-trimethoxy-1-naphthyl, 2,4,8-trimethoxy-1-naphthyl, 2-nitrophenyl, 3-nitrophenyl, 4-nitrophenyl, 3,5-dinitrophenyl, 2,5-dinitrophenyl, 2,6-dinitrophenyl, 2,4-dinitrophenyl, 2,3,6-trinitrophenyl, 2,3,4-trinitrophenyl, 3,4,5-trinitrophenyl, 2,5,6-trinitrophenyl, 2,4,6-trinitrophenyl, 1-nitro-2-naphthyl, 2-nitro-1-naphthyl, 3-nitro-1-naphthyl, 3,8-dinitro-1-naphthyl, 2,3-dinitro-1-naphthyl, 4,8-dinitro-1-naphthyl, 5,6-dinitro-1-naphthyl, 2,3,6-trinitro-1-naphthyl, 2,3,4-trinitro-1-naphthyl, 3,4,5-trinitro-1-naphthyl, 4,5,6-trinitro-1-naphthyl and 2,4,8-trinitro-1-naphthyl.

A preferable ether residue of ether derivatives is selected from the group consisting of a C₁-C₂₁ alkyl group; a C₂-C₂₁ alkenyl or alkynyl group having 1 to 3 double or triple bonds; a C₁-C₂ a alkyl group which has 1 to 4 substituents selected from the group consisting of lower alkoxy, halo and nitro groups; a C₁-C₂₁ alkyl group which has 1 to 3 C₆-C₁₀ aryl groups which are optionally substituted with 1 to 4 substituents selected from the group consisting of lower alkyl lower alkoxy, halo and nitro groups; and a C₆-C₁₀ aryl group which is optionally substituted with 1 to 4 substituents selected from the group consisting of lower alkyl, lower alkoxy, halo and nitro groups.

A more preferable ether residue of ether derivatives is selected from the group consisting of a C₁-C₂₁ alkyl group; a C₂-C₂₁ alkenyl group having 1 to 3 double bonds; a C₂-C₆ alkynyl group having one triple bond; a C₁-C₆ alkyl group which has 1 to 4 substituents selected from the group consisting of C₁-C₄ alkoxy, halogen and nitro groups; a C₁-C₆ alkyl group which has 1 to 3 C₆-C₁₀ aryl groups which are optionally substituted with 1 to 3 substituents selected from the group consisting of C₁-C₄ alkyl, C1-C₄ alkoxy, halo and nitro groups; and a C₆-C₁₀ aryl group which is optionally substituted with 1 to 3 substituents selected from the group consisting of C₁-C₄ alkyl, C₁-C₄ alkoxy, halo and nitro groups.

A more preferable ether residue of ether derivatives is selected from the group consisting of a C₁-C₂₁ alkyl group; a C₆-C₂₀ alkenyl group having 1 to 3 double bonds; a C₂-C₆ alkynyl group having one triple bond; a C₁-C₆ alkyl group which has one substituent selected from the group consisting of C₁-C₄ alkoxy and nitro groups; a C₁-C₆ alkyl group which has 1 to 3 substituents selected from the group consisting of halo groups; a C₁-C₄ alkyl group which has 1 to 3 phenyl or naphthyl groups which are optionally substituted with 1 to 3 substituents selected from the group consisting of C₁-C₄ alkyl, C₁-C₄alkoxy, halo and nitro groups; and a phenyl or naphthyl group which is optionally substituted with 1 to 3 substituents selected from the group consisting of C₁-C₄ alkyl, C₁-C₄ alkoxy, halo and nitro groups.

A more preferable ether residue of ether derivatives is selected from the group consisting of a C₆-C₂₀ alkyl group; a C₁₀-C₂₀ alkenyl group having 1 to 3 double bonds; a C₃-C₅ alkynyl group having one triple bond; a C₁-C₄ alkyl group which has one substituent selected from the group consisting of C₁-C₄ alkoxy and nitro group; a C₁-C₄ alkyl group which has 1 to 3 substituents selected from the group consisting of fluoro and chloro groups; a C₁-C₄ alkyl group which has 1 to 3 phenyl groups which are optionally substituted with 1 or 2 substituents selected from the group consisting of C₁-C₂ alkyl, C₁-C₄ alkoxy, fluoro and chloro group; and a phenyl group which is optionally substituted with 1 to 3 substituents selected from the group consisting of C₁-C₂ alkyl, C₁-C₄ alkoxy, fluoro and chloro groups.

A more preferable ether residue of ether derivatives is selected from the group consisting of a C₆-C₂₀ alkyl group: a C₁₀-C₂₀ alkenyl group having 1 to 3 double bonds; a C₃-C₅ alkynyl group having one triple bond; a C₁-C₄ alkyl group which has one substituent selected from the group consisting of C₁-C₄ alkoxy, fluoro, chloro and nitro groups; a C₁-C₄ alkyl group which has 1 to 3 phenyl groups which are optionally substituted with 1 or 2 substituents selected from the group consisting of C₁-C₂ alkyl, C₁-C₄ alkoxy, fluoro and chloro groups; and a phenyl group which is optionally substituted with 1 to 3 substituents selected from the group consisting of C₁-C₂ alkyl, C₁-C₄ alkoxy, fluoro and chloro groups.

A still more preferable ether residue of ether derivative is selected from the group consisting of a C₆-C₂₀ alkyl group: a C₁₀-C₂₀ alkenyl group having 1 to 3 double bonds; a C₃-C₅ alkynyl group having one triple bond; a C₁-C₄ alkyl group which has one substituent selected from the group consisting of C₁-C₄ alkoxy groups; and a C₁-C₄ alkyl group which has 1 or 2 phenyl groups optionally substituted with 1 or 2 substituents selected from the group consisting of C₁-C₂ alkyl, C₁-C₄ alkoxy, fluoro and chloro groups.

A most preferable ether residue of ether derivatives is selected from the group consisting of a C₆-C₂₀ alkyl group and a C₁₀-C₂₀ alkenyl group having 1 to 3 double bonds.

An alkyl residue of N-alkylcarbamoyl derivatives is selected from the group consisting of straight or branched chain C₁-C₂₁ alkyl group, such as methyl, ethyl, propyl, isopropyl, butyl, isobutyl, sec-butyl, tert-butyl, pentyl, isopentyl, 2-methylbutyl, neopentyl, 1-ethylpropyl, hexyl, isohexyl, 4-methylpentyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, heptyl, 1-methylhexyl, 2-methylhexyl, 3-methylhexyl, 4-methylhexyl, 5-methylhexyl, 1-propylbutyl, 4,4-dimethylpentyl, octyl, 1-methylheptyl, 2-methylheptyl, 3-methylheptyl, 4-methylheptyl, 5-methylheptyl, 6-methylheptyl, 1-propylpentyl, 2-ethylhexyl, 5,5-dimethylhexyl, nonyl, 3-methyloctyl, 4-methyloctyl, 5-methyloctyl, 6-methyloctyl, 1-propylhexyl, 2-ethylheptyl, 6,6-dimethylheptyl, decyl, 1-methylnonyl, 3-methylnonyl, 8-methylnonyl, 3-ethyloctyl, 3,7-dimethyloctyl, 7,7-dimethyloctyl, undecyl, 4,8-dimethylnonyl, dodecyl, tridecyl, tetradecyl, pentadecyl, 3,7,11-trimethyldodecyl, hexadecyl, 4,8,12-trimethyltridecyl, 1-methylpentadecyl, 14-methylpentadecyl, 13,13-dimethyltetradecyl, heptadecyl, 15-methylhexadecyl, octadecyl, 1-methylheptadecyl, nonadecyl, icosyl, 3,7,11,15-tetramethylhexadecyl and henicosyl groups;

straight or branched chain C₂-C₂₁ alkenyl or alkynyl group, such as ethenyl, 1-propenyl, 2-propenyl, 1-methyl-2-propenyl, 1-methyl-1-propenyl, 2-methyl-1-propenyl, 2-methyl-2-propenyl, 2-ethyl-2-propenyl, 1-butenyl, 2-butenyl, 1-methyl-2-butenyl, 1-methyl-1-butenyl, 3-methyl-2-butenyl, 1-ethyl-2-butenyl, 3-butenyl, 1-methyl-3-butenyl, 2-methyl-3-butenyl, 1-ethyl-3-butenyl, 1-pentenyl, 2-pentenyl, 1-methyl-2-pentenyl, 2-methyl-2-pentenyl, 3-pentenyl, 1-methyl-3-pentenyl, 2-methyl-3-pentenyl, 4-pentenyl, 1-methyl-4-pentenyl, 2-methyl-4-pentenyl, 1-hexenyl, 2-hexenyl, 3-hexenyl, 4-hexenyl, 5-hexenyl, cis-8-heptadecenyl, cis, cis-8,11-heptadecadienyl, cis, cis, cis-8,11,14-heptadecatrienyl, cis-10-nonadecenyl, cis-12-icosenyl, ethynyl, 2-propynyl, 1-methyl-2-propynyl, 2-methyl-2-propynyl, 2-ethyl-2-propynyl, 2-butynyl, 1-methyl-2-butynyl, 2-methyl-2-butynyl, 1-ethyl-2-butynyl, 3-butynyl, 1-methyl-3-butynyl, 2-methyl-3-butynyl, 1-ethyl-3-butynyl, 2-pentynyl, 1-methyl-2-pentynyl, 2-methyl-2-pentynyl, 3-pentynyl, 1-methyl-3-pentynyl, 2-methyl-3-pentynyl, 4-pentynyl, 1-methyl-4-pentynyl, 2-methyl-4-pentynyl, 2-hexynyl, 3-hexynyl, 4-hexynyl and 5-hexynyl;

straight or branched chain C₁-C₂₁ alkyl group which has substituents selected from the group consisting of alkoxy, halogen (hereinafter example fluorine, chlorine, bromine and iodine, preferably fluorine and chlorine) and nitro, such as methoxymethyl, ethoxymethyl, methoxyethyl, ethoxyethyl, trifluoromethyl, trichloromethyl, difluoromethyl, dichloromethyl, dibromomethyl, fluoromethyl, 2,2,2-trifluoroethyl, 2,2,2-trichloroethyl, 2-bromoethyl, 2-chloroethyl, 2-fluoroethyl, 2-iodoethyl, 3-chloropropyl, 4-fluorobutyl, 6-iodohexyl, 2,2-dibromoethyl, nitromethyl, dinitromethyl, 1-nitroethyl, 2-nitroethyl and 1,2-dinitroethyl; and

(C₆-C₁₀)aryl-(C₁-C₂₁)alkyl group wherein said aryl moiety optionally has substituent selected from the group consisting of lower alkyl, lower alkoxy, halogen and nitro groups, such as benzyl, α-naphthylmethyl, β-naphthylmethyl, indenylmethyl, phenanthrenylmethyl, anthracenylmethyl, diphenylmethyl, triphenylmethyl, 1-phenethyl, 2-phenethyl, 1-naphthylethyl, 2-naphthylethyl, 1-phenylpropyl, 2-phenylpropyl, 3-phenylpropyl, 1-naphthylpropyl, 2-naphthylpropyl, 3-naphthylpropyl, 1-phenylbutyl, 2-phenylbutyl, 3-phenylbutyl, 4-phenylbutyl, 1-naphthylbutyl, 2-naphthylbutyl, 3-naphthylbutyl, 4-naphthylbutyl, 1-phenylpentyl, 2-phenylpentyl, 3-phenylpentyl, 4-phenylpentyl, 5-phenylpentyl, 1-naphthylpentyl, 2-naphthylpentyl, 3-naphthylpentyl, 4-naphthylpentyl, 5-naphthylpentyl, 1-phenylhexyl, 2-phenylhexyl, 3-phenylhexyl, 4-phenylhexyl, 5-phenylhexyl, 6-phenylhexyl, 1-naphthylhexyl, 2-naphthylhexyl, 3-naphthylhexyl, 4-naphthylhexyl, 5-naphthylhexyl and 6-naphthylhexyl.

A preferable alkyl residue of N-alkylcarbamoyl derivatives is selected from the group consisting of a C₁-C₂₁ alkyl group; a C₂-C₂₁ alkenyl or alkynyl group having 1 to 3 double or triple bonds; a C₁-C₂₁ alkyl group which has one or more substituents selected from the group consisting of lower alkoxy, halo and nitro groups; and a C₁-C₂₁ alkyl group which has 1 to 3 C₆-C₁₀ aryl groups which are optionally substituted with 1 to 4 substituents selected from the group consisting of lower alkyl, lower alkoxy, halo and nitro groups.

A more preferable alkyl residue of N-alkylcarbamoyl derivatives is selected from the group consisting of a C₁-C₂₁ alkyl group; a C₂-C₂₁ alkenyl group having 1 to 3 double bonds; a C₂-C₆ alkynyl group having one triple bond; a C₁-C₆ alkyl group which has 1 to 4 substituents selected from the group consisting of C₁-C₄ alkoxy, halogen and nitro groups; and a C₁-C₆ alkyl group which has 1 to 3 C₆-C₁₀ aryl groups which are optionally substituted with 1 to 3 substituents selected from the group consisting of C₁-C₄ alkyl, C₁-C₄ alkoxy, halo and nitro groups.

A more preferable alkyl residue of N-alkylcarbamoyl derivatives is selected from the group consisting of a C₁-C₂₁ alkyl group; a C₆-C₂₀ alkenyl group having 1 to 3 double bonds; a C₂-C₆ alkynyl group having one triple bond; a C₁-C₆ alkyl group which has one substituent selected from the group consisting of C₁-C₄ alkoxy and nitro groups; a C₁-C₆ alkyl group which has 1 to 3 substituents selected from the group consisting of halo group; and a C₁-C₄ alkyl group which has 1 to 3 phenyl or naphthyl groups which are optionally substituted with 1 to 3 substituents selected from the group consisting of C₁-C₄ alkyl, C₁-C₄ alkoxy, halo and nitro groups.

A more preferable alkyl residue of N-alkylcarbamoyl derivatives is selected from the group consisting of a C₆-C₂₀ alkyl group; a C₁₀-C₂₀ alkenyl group having 1 to 3 double bonds; a C₃-C₅ alkynyl group having one triple bond: a C₁-C₄ alkyl group which has one substituent selected from the group consisting of C₁-C₄ alkoxy and nitro groups; a C₁-C₄ alkyl group which has 1 to 3 substituents selected from the group consisting of fluoro and chloro groups; and a C₁-C₄ alkyl group which has 1 to 3 phenyl groups which are optionally substituted with 1 to 2 substituents selected from the group consisting of C₁-C₂ alkyl, C₁-C₄ alkoxy, fluoro and chloro groups.

A more preferable alkyl residue of N-alkylcarbamoyl derivatives is selected from the group consisting of a C₆-C₁₀ alkyl group, a C₁₀-C₂₀ alkenyl group having 1 to 3 double bonds; a C₃-C₅ alkynyl group having one triple bond; a C₁-C₄ alkyl group which has one substituent selected from the group consisting of C₁-C₄ alkoxy, fluoro, chloro and nitro groups, and a C₁-C₄ alkyl group which has 1 to 3 phenyl groups which are optionally substituted with 1 to 2 substituents selected from the group consisting of C₁-C₂ alkyl, C₁-C₄ alkoxy, fluoro and chloro groups.

A still more preferable alkyl residue of N-alkylcarbamoyl derivative is selected from the group consisting of a C₆-C₂₀ alkyl group; a C₁₀-C₂₀ alkenyl group having 1 to 3 double bonds; a C₃-C₅ alkynyl group having one triple bond; a C₁-C₄ alkyl group which has one substituent selected from the group consisting of C₁-C₄ alkoxy groups; and a C₁-C₄ alkyl group which has 1 to 2 phenyl groups optionally substituted with 1 to 2 substituents selected from the group consisting of C₁-C₂ alkyl, C₁-C₄ alkoxy, fluoro and chloro groups.

A most preferable alkyl residue of N-alkylcarbamoyl derivatives is selected from the group consisting of a C₆-C₂₀ alkyl group and a C₁₀-C₂₀ alkenyl group having 1 to 3 double bonds.

In compounds of Formulae I, Ia, Ib, I-A, II, Ia or IIb, there are several functional groups to which the hydroxy protecting group, and the ester, ether and alkyl residues can be attached. Therefore a plurality of protecting groups and residues can independently exist by optional combination of these protecting groups and residues.

A preferable pharmaceutically acceptable ester derivative is a derivative which has one or two of the ester residues at R², R^{2a}, R³, R⁴, R^{4a} and/or R⁵. A more preferable ester derivative is a derivative which has one or two of the ester residues at R³ and/or R⁵. A still more preferable ester derivative is a derivative which has one of the ester residues at R³ or R⁵. A most preferable ester derivative is a derivative which has one of the ester residue at R³.

A preferable pharmaceutically acceptable ether derivative is a derivative which has one or two of the ether residues at R², R^{2a}, R³, R⁴, R^{4a} and/or R⁵. A more preferable ether derivative is a derivative which has one or two of the ether residues at R³ and/or R⁵. A still more preferable ether derivative is a derivative which has one of the ether residues at R³ or R⁵. A most preferable ether derivative is a derivative which has one of the ether residues at R³.

A preferable pharmaceutically acceptable N-alkylcarbamoyl derivative is a derivative having one of the alkyl residues.

The term “pharmaceutically acceptable salt” refers to a salt that is a useful medicament without significant toxicity.

Where compounds of Formula I, Ia, Ib, I-A, II, Ia or IIb, and pharmaceutically acceptable ester, ether and N-alkyl derivatives of compound Formula I, Ia, Ib, I-A, II, IIa or IIb have a basic group such as an amino group these compounds can be converted into an acid addition salt by a conventional treatment with an acid. Such acid addition salts include inorganic acid salts such as hydrochloride, hydrobromide, sulfate and phosphate; organic acid salts such as acetate, benzoate, oxalate. maleate, fumarate, tartrate and citrate; and sulfonic acid salts such as methanesulfonate, benzenesulfonate and p-toluenesulfonate.

Where compounds of Formula I, Ia, Ib, I-A, II, IIa or IIb and pharmaceutically acceptable ester, ether and N-alkyl derivatives of the compounds have an acidic group such as a carboxy group, these compounds can be converted into a base addition salt by a conventional treatment with a base. Such base addition salts include alkali metal salts such as sodium, potassium and lithium salts; alkaline earth metal salts such as calcium and magnesium salts; metal salts such as aluminium, iron, zinc, copper, nickel and cobalt salts; and quaternary ammonium salts such as ammonium salt.

When compounds of Formula I, Ia, Ib, I-A, II, IIa or IIb and pharmaceutically acceptable derivative of compounds are allowed to stand in the atmosphere, these compounds may take up water to form a hydrate. The present invention includes such hydrates. Compounds of Formula I, Ia, Ib, I-A, II, Ia or IIb and pharmaceutically acceptable derivative of compounds may absorb a solvent to form a solvate. The present invention includes such solvates.

Compounds of Formula I, Ia, Ib, I-A, II, Ia or IIb and pharmaceutically acceptable derivative of compounds have several asymmetric carbons and therefore they can exist as several stereoisomers such as enantiomers and diastereomers in which each carbon has R or S configuration. The compound of the present invention encompasses individual enantiomers and diastereomers and mixtures of these stereoisomers in all proportions.

A preferable configuration of the compounds of formulae I, Ia, Ib, I-A, II, IIa or IIb of the present invention are shown below as formulae I′, I-A′, Ia′, Ib′, II′, IIa′, and IIb′, where R¹, X, X¹, X², R², R^{2a}, R³, R^{4a} and R⁵ are as defined above for the corresponding formula.

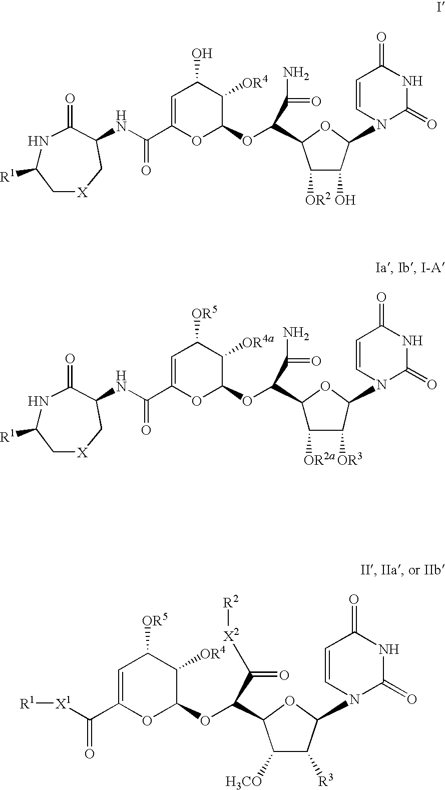

A preferable compound (I) is selected from the following compounds or pharmaceutically acceptable ethers or esters thereof: (1) a compound (I) wherein R² is a methyl group, (2) a compound (I) wherein R⁴ is hydrogen, (3) a compound (I) wherein X is a methylene group; or a compound wherein R², R⁴ and X is selected in optional combination of (1), (2) and (3), for example: (4) a compound (I) wherein R⁴ is hydrogen and X is a methylene group, and (5) a compound (I) wherein R² is a methyl group, R⁴ is hydrogen and X is a methylene group.

Preferable compounds of Formulae I, Ia, Ib, I-A, II, IIa or IIb are selected from the following compounds or pharmaceutically acceptable ethers or esters thereof: (i) compounds of Formulae I, Ia, Ib, I-A, II, IIa or IIb wherein the protecting group for a hydroxy group is selected from the group consisting of “tetrahydropyranyl or tetrahydrothiopyranyl group”, “silyl group”, “aralkyl group”, “aralkyloxycarbonyl group”, “1-(aliphatic acyloxy)-(lower alkyl) group”, “1-(cycloalkylcarbonyloxy)-(lower alkyl) group”, “1-(lower alkoxycarbonyloxy)-(lower alkyl) group”, “1-(cycloalkyloxycarbonyloxy)-(lower alkyl) group”, “phthalidyl” and “oxodioxolenylmethyl group”. (ii) compounds of Formulae I, Ia, Ib, I-A, II, IIa or IIb wherein the protecting group for a hydroxy group is selected from the group consisting of tetrahydropyran-2-yl, 4-methoxytetrahydropyran-4-yl, tetrahydrothiopyran-2-yl, trimethylsilyl, triethylsilyl, tert-butyldimethylsilyl, di(tert-butyl)methylsilyl, diphenylmethylsilyl, benzyl, diphenylmethyl, triphenylmethyl, 4-methylbenzyl, 4-methoxybenzyl, 2-nitrobenzyl, 4-nitrobenzyl, 4-chlorobenzyl, benzyloxycarbonyl, 4-methoxybenzyloxycarbonyl, 2-nitrobenzyloxycarbonyl, 4-nitrobenzyloxycarbonyl, acetoxymethyl, propionyloxymethyl, butyryloxymethyl, pivaloyloxymethyl, valeryloxymethyl, 1-acetoxyethyl, butyryloxyethyl, 1-pivaloyloxyethyl, cyclopentylcarbonyloxymethyl, cyclohexylcarbonyloxymethyl, 1-cyclopentylcarbonyloxyethyl, 1-cyclohexylcarbonyloxyethyl, methoxycarbonyloxymethyl, ethoxycarbonyloxymethyl, propoxycarbonyloxymethyl, isopropoxycarbonyloxymethyl, butoxycarbonyloxymethyl, isobutoxycarbonyloxymethyl, 1-(methoxycarbonyloxy)ethyl, 1-(ethoxycarbonyloxy)ethyl, 1-(isopropoxycarbonyloxy)ethyl, cyclopentyloxycarbonyloxymethyl, cyclohexyloxycarbonyloxymethyl, 1-(cyclopentyloxycarbonyloxy)ethyl, 1-(cyclohexyloxycarbonyloxy)ethyl, phthalidyl, (5-phenyl-2-oxo-1,3-dioxolen-4-yl)methyl, [5-(4-methylphenyl)-2-oxo-1,3-dioxolen-4-yl]methyl, (5-methyl-2-oxo-1,3-dioxolen-4-yl)methyl and (5-ethyl-2-oxo-1,3-dioxolen-4-yl)methyl group. (iii) compounds of Formulae I, Ia, Ib, I-A, II, IIa or IIb wherein the protecting group of hydroxy group is selected from the group consisting of trimethylsilyl, tert-butyldimethylsilyl, triphenylmethyl, benzyl, 4-methoxybenzyl, acetoxymethyl, propionyloxymethyl, butyryloxymethyl, pivaloyloxymethyl, valeryloxymethyl, cyclopentylcarbonyloxymethyl, cyclohexylcarbonyloxymethyl, methoxycarbonyloxymethyl, ethoxycarbonyloxymethyl, propoxycarbonyloxymethyl, isopropoxycarbonyloxymethyl, butoxycarbonyloxymethyl, isobutoxycarbonyloxymethyl, cyclopentyloxycarbonyloxymethyl, cyclohexyloxycarbonyloxymethyl, (5-phenyl-2-oxo-1,3-dioxolen-4-yl)methyl, [5-(4-methylphenyl)-2-oxo-1,3-dioxolen-4-yl]methyl, (5-methyl-2-oxo-1,3-dioxolen-4-yl)methyl and (5-ethyl-2-oxo-1,3-dioxolen-4-yl)methyl group.

A preferable ester derivative of compounds of Formula I, Ia, Ib, I-A, II, IIa or IIb is selected from the following compounds: (iv) an ester derivative of compounds of Formula I, Ia, Ib, I-A, II, IIa or IIb wherein the ester residue is R⁶ CO— or R⁶ OCO— group in which R⁶ is selected from the group consisting of hydrogen; a C1-C₂₁ alkyl group; a C₂-C₂₁ alkenyl or alkynyl group having 1 to 3 double or triple bonds; a C₁-C₂₁ alkyl group substituted with 1 to 4 substituents selected from the group consisting of lower alkoxy, halo and nitro groups; a C₁-C₂₁ alkyl group substituted with 1 to 3 C₆-C₁₀ aryl groups which are optionally substituted with 1 to 4 substituents selected from the group consisting of lower alkyl, lower alkoxy, halo and nitro groups; and a C₆-C₁₀ aryl group which is optionally substituted with 1 to 4 substituents selected from the group consisting of lower alkyl, lower alkoxy, halo and nitro groups. (v) an ester derivative of compounds of Formula I, Ia, Ib, I-A, II, IIa or IIb wherein the ester residue is R⁶ CO— or R⁶ OCO— group in which R⁶ is selected from the group consisting of hydrogen; a C₁-C₂₁ alkyl group; a C₂-C₂₁ alkenyl group having 1 to 3 double bonds; a C₂-C₆ alkynyl group having one triple bond; a C₁-C₆ alkyl group substituted with 1 to 4 substituents selected from the group consisting of C₁-C₄ alkoxy, halo and nitro groups; a C₁-C₆ alkyl group substituted with 1 to 3 C₆-C₁₀ aryl groups which are optionally substituted with 1 to 3 substituents selected from the group consisting of C₁-C₄ alkyl, C₁-C₄ alkoxy, halo and nitro groups; and a C₆-C₁₀ aryl group which is optionally substituted with 1 to 3 substituents selected from the group consisting of C₁-C₄ alkyl C₁-C₄ alkoxy, halo and nitro groups. (vi) an ester derivative of compounds of Formula I, Ia, Ib, I-A, II, IIa or IIb wherein the ester residue is R⁶ CO— or R⁶ OCO— group in which R⁶ is selected from the group consisting of a C₁-C₂, alkyl group; a C₆-C₂₀ alkenyl group having 1 to 3 double bonds; a C₂-C₆ alkynyl group having one triple bond; a C₁-C₆ alkyl group substituted with one substituent selected from the group consisting of C₁-C4 alkoxy and nitro groups; a C₁-C₆ alkyl group substituted with 1 to 3 substituents selected from the group consisting of halogen; a C₁-C₄ alkyl group substituted with 1 to 3 phenyl or naphthyl groups which are optionally substituted with 1 to 3 substituents selected from the group consisting of C₁-C₄ alkyl, C₁-C₄ alkoxy, halo and nitro groups; and a phenyl or naphthyl group which is optionally substituted with 1 to 3 substituents selected from the group consisting of C₁-C₄ alkyl, C₁-C₄ alkoxy, halo and nitro groups. (vii) an ester derivative of compounds of Formula I, Ia, Ib, I-A, II, IIa or IIb wherein the ester residue is R⁶ CO— or R⁶ OCO— group in which R⁶ is selected from the group consisting of C₆-C₂₀ alkyl group; a C₁₀-C₂₀ alkenyl group having 1 to 3 double bonds; a C₃-C₅ alkynyl group having one triple bond; a C₁-C₄ alkyl group substituted with one substituent selected from the group consisting of C₁-C₄ alkoxy, and nitro groups: a C₁-C₄ alkyl group substituted with 1 to 3 substituents selected from the group consisting of fluoro and chloro groups; a C₁-C₄ alkyl group substituted with 1 to 3 phenyl groups which are optionally substituted with 1 or 2 substituents selected from the group consisting of C₁-C₂ alkyl, C₁-C₄ alkoxy, fluoro, and chloro groups; and a phenyl group which is optionally substituted with 1 to 3 substituents selected from the group consisting of C₁-C₂ alkyl, C₁-C₄ alkoxy, fluoro and chloro groups. (viii) an ester derivative of compounds of Formula I, Ia, Ib, I-A, II, IIa or IIb wherein the ester residue is R⁶ CO— or R⁶ OCO— group in which R⁶ is selected from the group consisting of a C₆-C₂₀ alkyl group; a C₁₀-C₂₀ alkenyl group having 1 to 3 double bonds; a C₃-C₅ alkynyl group having one triple bond; a C₁-C₄ alkyl group substituted with one substituent selected from the group consisting of C₁-C₄ alkoxy, fluoro, chloro and nitro groups a C₁-C₄ alkyl group substituted with 1 to 3 phenyl groups which are optionally substituted with 1 or 2 substituents selected from the group consisting of C₁-C₂ alkyl, C₁-C₄ alkoxy, fluoro, and chloro groups; and a phenyl group which is optionally substituted with 1 to 3 substituents selected from the group consisting of C₁-C₂ alkyl, C₁-C₄ alkoxy, fluoro and chloro groups. (ix) an ester derivative of compounds of Formula I, Ia, Ib, I-A, II, IIa or IIb wherein the ester residue is R⁶ CO— or R⁶ OCO— group in which R⁶ is selected from the group consisting of a C₆-C₂₀ alkyl group; a C₁₀-C₂₀ alkenyl group having 1 to 3 double bonds; a C₃-C₅ alkynyl group having one triple bond; a C₁-C₄ alkyl group substituted with one substituent selected from the group consisting of C₁-C₄ alkoxy groups; and a C₁-C₄ alkyl group substituted with 1 to 2 phenyl groups which are optionally substituted with 1 or 2 substituents selected from the group consisting of C₁-C₂ alkyl, C₁-C₄ alkoxy, fluoro and chloro groups. (x) an ester derivative of compounds of Formula I, Ia, Ib, I-A, II, IIa or IIb wherein the ester residue is R⁶ CO— or R⁶ OCO— group in which R⁶ is selected from the group consisting of a C₆-C₂₀ alkyl group; and a C₁₀-C₂₀ alkenyl group having 1 to 3 double bonds.

A preferable ether derivative of compounds of Formula I, Ia, Ib, I-A, II, IIa or IIb is selected from following compounds: (xi) an ether derivative of compound of Formulae I, Ia, Ib, I-A, II, IIa or IIb wherein the ether residue is selected from the group consisting of a C₁-C₂₁ alkyl group; a C₂-C₂₁ alkenyl or alkynyl group having 1 to 3 double or triple bonds; a C₁-C₂₁ alkyl group which has 1 to 3 substituents selected from the group consisting of lower alkoxy, halo and nitro groups; a C₁-C₂₁ alkyl group which has 1 to 3 C₆-C₁₀ aryl groups which are optionally substituted with 1 to 4 substituents selected from the group consisting of lower alkyl, lower alkoxy, halo and nitro groups; and a C₆-C₁₀ aryl group which is optionally substituted with 1 to 4 is substituents selected from the group consisting of lower alkyl, lower alkoxy, halo and nitro groups. (xii) an ether derivative of compounds of Formulae I, Ia, Ib, I-A, II, IIa or IIb wherein the ether residue is selected from the group consisting of a C₁-C₂₁ alkyl group; a C₂-C₂₁ alkenyl group having 1 to 3 double bonds; a C₂-C₆ alkynyl group having one triple bond; a C₁-C₆ alkyl group which has 1 to 4 substituents selected from the group consisting of C₁-C₄ alkoxy, halo and nitro group; a C₁-C₆ alkyl group which has 1 to 3 C₆-C₁₀ aryl groups which is optionally substituted with 1 to 3 substituents selected from the group consisting of C₁-C₄ alkyl, C₁-C₄ alkoxy, halo and nitro groups; and a C₆-C₁₀ aryl group which are optionally substituted with 1 to 3 substituents selected from the group consisting of C₁-C₄ alkyl, C₁-C₄ alkoxy, halo and nitro groups. (xiii) an ether derivative of compounds of Formulae I, Ia, Ib, I-A, II, IIa or IIb wherein the ether residue is selected from the group consisting of a C₁-C₂₁ alkyl group; C₆-C₂₀ alkenyl group having 1 to 3 double bonds; a C₂-C₆ alkynyl group having one triple bond; a C₁-C₆ alkyl group which has one substituent selected from the group consisting of C₁-C₄ alkoxy and nitro groups; C₁-C₆ alkyl group which has 1 to 3 substituents selected from the group consisting of halo group; a C₁-C₄ alkyl group which has 1 to 3 phenyl or naphthyl groups which are optionally substituted with 1 to 3 substituents selected from the group consisting of C₁-C₄ alkyl, C₁-C₄ alkoxy, halogen and nitro groups; and a phenyl or naphthyl group which is optionally substituted with 1 to 3 substituents selected from the group consisting of C₁-C₄ alkyl, C₁-C₄ alkoxy, halo and nitro groups. (xiv) an ether derivative of compounds of Formulae I, Ia, Ib, I-A, II, IIa or IIb wherein the ether residue is selected from the group consisting of a C₆-C₂₀ alkyl group a C₁₀-C₂₀ alkenyl group having 1 to 3 double bonds; a C₃-C₅ alkynyl group having one triple bond; a C₁-C₄ alkyl group which has one substituent selected from the group consisting of C₁-C₄ alkoxy and nitro groups; a C₁-C₄ alkyl group which has 1 to 3 substituents selected from the group consisting of fluoro and chloro groups; a C₁-C₄ alkyl group which has 1 to 3 phenyl groups which are optionally substituted with 1 or 2 substituents selected from the group consisting of C₁-C₂ alkyl, C₁-C₄ alkoxy, fluoro and chloro groups; and a phenyl group which is optionally substituted with 1 to 3 substituents selected from the group consisting of C₁-C₂ alkyl, C₁-C₄ alkoxy, fluoro and chloro groups. (xv) an ether derivative of compounds of Formulae I, Ia, Ib, I-A, II, IIa or IIb wherein the ether residue is selected from the group consisting of a C₆-C₂₀ alkyl group; a C₁₀-C₂₀ alkenyl group having 1 to 3 double bonds; a C₃-C₅ alkynyl group having one triple bond; a C₁-C₄ alkyl group which has one substituent selected from the group consisting of C₁-C₄ alkoxy, fluoro, chloro, and nitro groups; a C₁-C₄ alkyl group which has 1 to 3 phenyl groups which are optionally substituted with 1 or 2 substituents selected from the group consisting of C₁-C₂ alkyl, C1-C4 alkoxy, fluoro and chloro groups, and a phenyl group which is optionally substituted with 1 to 3 substituents selected from the group consisting of C₁-C₂ alkyl, C₁-C₄ alkoxy, fluoro and chloro groups. (xvi) an ether derivative of compounds of Formulae I, Ia, Ib, I-A, II, IIa or IIb wherein the ether residue is selected from the group consisting of a C₆-C₂₀ alkyl group; a C₁₀-C₂₀ alkenyl group having 1 to 3 double bonds; a C₃-C₅ alkynyl group having one triple bond; a C₁-C₄ alkyl group which has one substituent selected from the group consisting of C₁-C₄ alkoxy group; and a C₁-C₄ alkyl group which has 1 or 2 phenyl groups optionally substituted with 1 or 2 substituents selected from the group consisting of C₁-C₄ alkyl, C1-C4 alkoxy, fluoro and chloro groups. (xvii) an ether derivative of compounds of Formulae I, Ia, Ib, I-A, II, IIa or IIb wherein the ether residue is selected from the group consisting of a C₆-C₂₀ alkyl group and a C₁₀-C₂₀ alkenyl group having 1 to 3 double bonds.

A preferable N-alkylcarbamoyl derivative of compounds of Formulae I, Ia, Ib, I-A, II, IIa or IIb is selected from the following compounds: (xviii) an N-alkylcarbamoyl derivative of compounds of Formulae I, Ia, Ib, I-A, II, IIa or IIb wherein the alkyl residue of the N-alkylcarbamoyl derivative is selected from the group consisting of a C₁-C₂₁ alkyl group; a C₂-C₂₁ alkenyl or alkynyl group having 1 to 3 double or triple bonds; a C₁-C₂₁ alkyl group which has 1 to 4 substituents selected from the group consisting of lower alkoxy, halo and nitro groups; and a C₁-C₂₁ alkyl group which has 1 to 3 C₆-C₁₀ aryl groups which are optionally substituted with 1 to 4 substituents selected from the group consisting of lower alkyl, lower alkoxy, halo and nitro groups. (xix) an N-alkylcarbamoyl derivative of compounds of Formulae I, Ia, Ib, I-A, II, IIa or IIb wherein the alkyl residue is selected from the group consisting of a C₁-C₂₁ alkyl group; a C₂-C₂₁ alkenyl group having 1 to 3 double bonds; a C₂-C₆ alkynyl group having one triple bond; a C₁-C₆ alkyl group which has 1 to 4 substituents selected from the group consisting of C₁-C₄ alkoxy, halo and nitro groups; and a C₁-C₆ alkyl group which has 1 to 3 C₆-C₁₀ aryl groups which are optionally substituted with 1 to 3 substituents selected from the group consisting of C₁-C₄ alkyl, C₁-C₄ alkoxy, halo and nitro group. (xx) an N-alkylcarbamoyl derivative of compounds of Formulae I, Ia, Ib, I-A, II, IIa or IIb wherein the alkyl residue is selected from the group consisting of a C₁-C₂₁ alkyl group; a C₆-C₂₀ alkenyl group having 1 to 3 double bonds; a C₂-C₆ alkynyl group having one triple bond; a C₁-C₆ alkyl group which has one substituent selected from the group consisting of C₁-C₄ alkoxy and nitro groups; a C₁-C₆ alkyl group which has 1 to 3 substituents selected from the group consisting of halo groups; and a C₁-C₄ alkyl group which has 1 to 3 phenyl or naphthyl groups which are optionally substituted with 1 to 3 substituents selected from the group consisting of C₁-C₄ alkyl, C₁-C₄ alkoxy, halo and nitro groups. (xxi) an N-alkylcarbamoyl derivative of compounds of Formulae I, Ia, Ib, I-A, II, IIa or IIb wherein the alkyl residue is selected from the group consisting of a C₆-C₂₀ alkyl group; a C₁₀-C₂₀ alkenyl group having 1 to 3 double bonds; a C₃-C₅ alkynyl group having one triple bond; a C₁-C₄ alkyl group which has one substituent selected from the group consisting of C₁-C₄ alkoxy and nitro groups; a C₁-C₄ alkyl group which has 1 to 3 substituents selected from the group consisting of fluoro and chloro groups; and a C₁-C₄ alkyl group which has 1 to 3 phenyl groups which are optionally substituted with 1 or 2 substituents selected from the group consisting of C₁-C₂ alkyl, C₁-C₄ alkoxy, fluoro and chloro groups. (xxii) an N-alkylcarbamoyl derivative of compounds of Formulae I, Ia, Ib, I-A, II, IIa or IIb wherein the alkyl residue is selected from the group consisting of a C₆-C₂₀ alkyl group; a C₁₀-C₂₀ alkenyl group having 1 to 3 double bonds; a C₃-C₅ alkynyl group having one triple bond; a C₁-C₄ alkyl group which has one substituent selected from the group consisting of C₁-C₄ alkoxy, fluoro, chloro and nitro groups; and a C₁-C₄ alkyl group which has 1 to 3 phenyl groups which are optionally substituted with 1 or 2 substituents selected from the group consisting of C₁-C₂ alkyl, C₁-C₄ alkoxy, fluoro and chloro groups. (xxiii) an N-alkylcarbamoyl derivative of compounds of Formulae I, Ia, Ib, I-A, II, IIa or IIb wherein the alkyl residue is selected from the group consisting of C₆-C₂₀ alkyl group; a C₁₀-C₂₀ alkenyl group having 1 to 3 double bonds; a C₃-C₅ alkynyl group having one triple bond; a C₁-C₄ alkyl group which has one substituent selected from the group consisting of C₁-C₄ alkoxy groups; and C₁-C₄ alkyl group which has 1 or 2 phenyl groups optionally substituted with 1 or 2 substituents selected from the group consisting of C₁-C₂ alkyl C₁-C₄ alkoxy, fluoro and chloro groups. (xxiv) an N-alkylcarbamoyl derivative of compounds of Formulae I, Ia, Ib, I-A, II, IIa or IIb wherein the alkyl residue is selected from the group consisting of a C₆-C₂₀ alkyl group and a C₁₀-C₂₀ alkenyl group having 1 to 3 double bonds.

More preferable compounds of Formulae I, Ia, Ib, I-A, II, IIa or IIb are selected from group (i) to (iii); group (iv) to (x); group (xi) to (xvii); group (xviii) to (xxiv) in optional combination of these groups, for example: (xxv) a compounds of Formulae I, Ia, Ib, I-A, II, IIa or IIb wherein the protecting group for a hydroxy group is (i) and the ester residue is (iv). (xxvi) a compounds of Formulae I, Ia, Ib, I-A, II, IIa or IIb wherein the protecting group for a hydroxy group is (ii) and the ester residue is (v). (xxvii) Compounds of Formulae I, Ia, Ib, I-A, II, IIa or IIb wherein the protecting group for a hydroxy group is (iii) and the ester residue is (vi). (xxviii) A compounds of Formulae I, Ia, Ib, I-A, II, IIa or IIb wherein the protecting group for a hydroxy group is (i) and the ether residue is (xi). (xxix) A compounds of Formulae I, Ia, Ib, I-A, II, IIa or IIb wherein the protecting group for a hydroxy group is (ii) and the ester residue is (xii). (xxx) A compounds of Formulae I, Ia, Ib, I-A, II, IIa or IIb wherein the protecting group for a hydroxy group is (iii) and the ether residue is (xiii). (xxxi) A compounds of Formulae I, Ia, Ib, I-A, II, IIa or IIb wherein the protecting group for a hydroxy group is (i) and the alkyl residue is (xviii). (xxxii) Compounds of Formulae I, Ia, Ib, I-A, II, IIa or IIb wherein the protecting group for a hydroxy group is (ii) and the alkyl residue is (xix). (xxxiii) A compounds of Formulae I, Ia, Ib, I-A, II, IIa or IIb wherein the protecting group for a hydroxy group is (iii) and the residue is (xx).

In preferred embodiments of the invention, formulations comprising the capuramycin analogues shown in Table 3 below are provided.

TABLE 3
Capuramycin analogues and IC₅₀ against translocase I

| | | IC₅₀ |
|---|---|---|
| Compound | Structure | (ng/ml) |
| SQ997 | 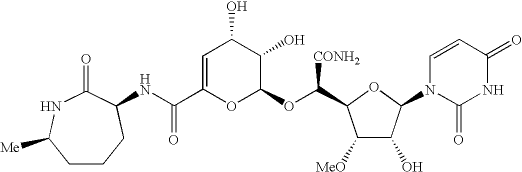 | 10 |
| SQ922 | 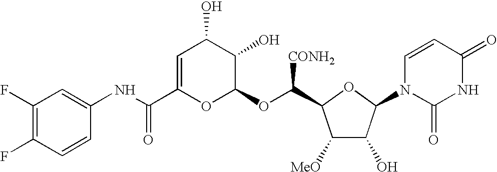 | 9 |
| SQ641 | 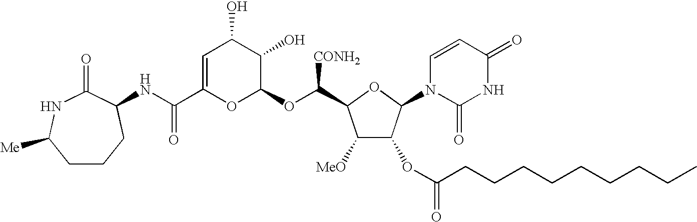 | 550 |
| RKS2243 | 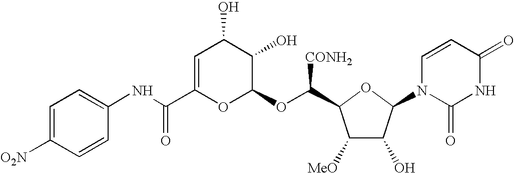 | 4 |
| RKS2244 | 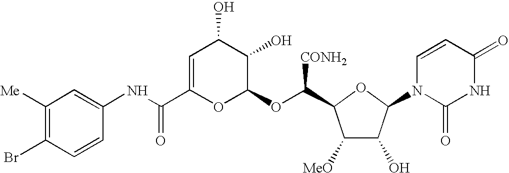 | 7.8 |
| RKS2186 | 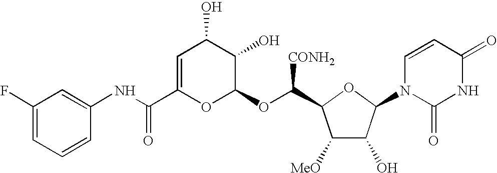 | 10 |
| RKS2241 | 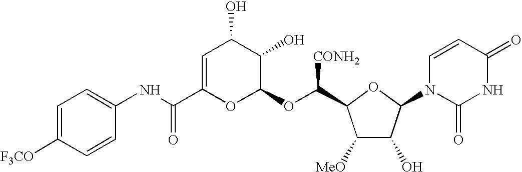 | 15 |
| RKS2235 | 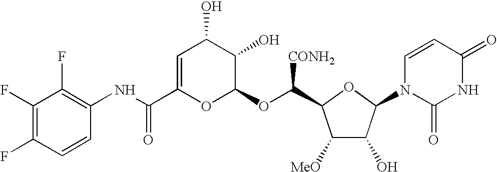 | 10 |
| RKS2033 | 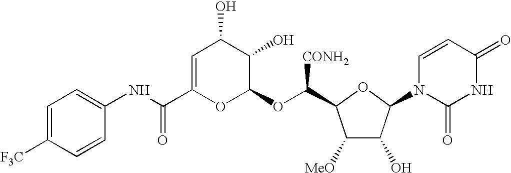 | 30 |
| RKS2137 | 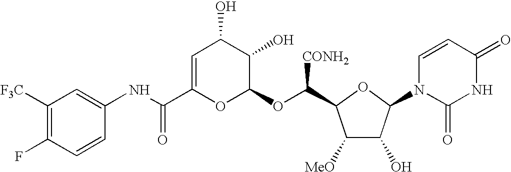 | 8 |
| RS-120029 | 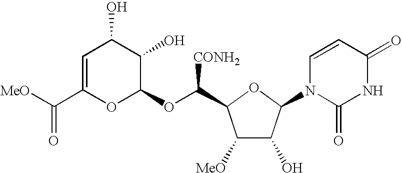 | |

Additional specific compounds of the invention represented by the embodiments presented above are known and may be found in Tables 1, 2 and 3 and in the complete disclosure of U.S. Pat. No. 6,472,384, and in Tables 1, 2 and 3 and the complete disclosure of U.S. Pat. Nos. 7,157,442 and 6,844,173, all of which are hereby incorporated by reference in their entirety. In addition, the patents describe methods for the preparation of the compounds from the culture broth of a microorganism of Streptomyces spp., preferably from the culture broth of Streptomyces griseus Strain SANK60196. Derivatives of the compounds are prepared by synthetic processes from precursor compounds isolated from the culture broths, as described in the patent.

Formulations with PEG Containing Compounds

Formulations of drugs with low aqueous solubility may be enhanced by including an organic compound that is derivatized with one or more residues of a hydrophilic polymer, including a polyethyleneglycol (PEG) residue or a residue of a block co-polymer of polyethylene glycol and polypropylene glycol known as poloxamers or by the trade name Pluronic (BASF, Germany). In some embodiments, thePEGylated compounds used in formulations include, but are not limited to, polyethoxylated castor oil, polyethylene glycol fatty acid esters, polyethoxylated vegetable oils, polyethoxylated lipophilic vitamins, polyethoxylated tocopherols, polyethoxylated tocotrienols, polyethoxylated cholesterol, polyethoxylated steroids, polyethoxylated vitamin E derivatives, and the like. The present invention provides novel formulations of capuramycin analogues with PEGylated compounds to improve solubility and intracellular adsorption and efficacy. It has been surprisingly been found that novel formulations of the capuramycin analogues described herein comprising PEGylated vitamin E derivatives exhibit remarkably improved solubility and intracellular adsorption and efficacy compared with standard formulations of the drugs. The improved intracellular profile may be due to decreased P-gp mediated drug efflux, which is achieved by the novel formulations of the compounds.

In one embodiment, the PEGylated compound comprises a hydrophobic organic compound that is derivatized with one or more a PEG residues. Hydrophobic compounds, as used herein, are compounds that are not soluble in water at room temperature (20-25° C.). Typically, hydrophobic compounds comprise hydrophobic groups such as alkyl chains or carbocyclic rings, including aryl or cycloalkyl rings. In one embodiment, the hydrophobic organic compound comprises one or more C₂-C₂₀ alkyl side chains, which may include one or more sites of unsaturation.

In one embodiment, the hydrophobic organic compound comprises a functionalized alkyl group wherein the alkyl group is functionalized with one or more hydroxy group, halogen, carboxyl group, amino group, alkyl or dialkylamino group, alkyl ester group, amide group, phosphate group, sulfonate group, or sulfate group.

In another embodiment, the hydrophobic organic compound comprises a C₆-C₁₄ aryl, heteroaryl, heterocyclic, or cycloalkyl ring. In another embodiment, the hydrophobic organic compound comprises a C₆-C₁₄ aryl, heteroaryl, heterocyclic, or cycloalkyl ring substituted with one or more C₂-C₂₀ alkyl side chains, which can include one or more sites of unsaturation. In still another embodiment, the hydrophobic organic compound comprises a C₆-C₁₄ aryl, heteroaryl, heterocyclic, or cycloalkyl ring substituted with one or more C₂-C₂₀ alkyl side chains, and one or more substituents including, but not limited to, an alkyl group, a hydroxy group, halogen, a carboxyl group, an amino group, an alkyl or dialkylamino group, an alkyl ester group, an amide group, a phosphate group, a sulfonate group, or a sulfate group. In another embodiment, the hydrophobic organic compound comprises a phenyl ring substituted with one or more C₂-C₂₀ alkyl side chains. In another embodiment, the hydrophobic organic compound comprises a naphthyl or anthracenyl ring substituted with one or more C₂-C₂₀ alkyl side chains. In another embodiment, the hydrophobic organic compound comprises a C₆-C₁₄ aryl, heteroaryl or cycloalkyl ring fused to a heterocyclic ring that is substituted with one or more C₂-C₂₀ alkyl side chains. In yet another embodiment, the hydrophobic organic compound comprises a C₆-C₁₄ aryl, heteroaryl or cycloalkyl ring substituted with one or more hydroxy, alkyl, carboxyl, amino, alkylamino, or dialkyl amino groups that is fused to heterocyclic ring, wherein the heterocyclic ring has 1-4 oxygen, nitrogen or sulfur atoms and is substituted with one or more C₂-C₂₀ alkyl side chains.

Examples of hydrophobic compounds that may be PEGylated include, but are not limited to, vitamin E and vitamin E derivatives, tocopherols, tocotrienols, sterols such as cholesterol, steroids, naphthaquinone derivatives, glycerides, diglycerides, triglycerides, lipids including saturated lipids, non-saturated lipids, synthetic lipids or lipids derived from natural sources, fat soluble vitamins and vitamin derivatives such as vitamin D₂ (ergocalciferol), vitamin D₃ (cholecalciferol), vitamin A (retinol), vitamin K, and the like. Lipids may include suitable phospholipids, phosphoglycerides, or other lipids. Hydrophobic compounds may also include phosphoglycerides including, but are not limited to, phosphatidylcholines, phosphatidylethanolamines, lysophosphatidylcholines, lysophosphatidylethanoloamines, phosphatidylserines, phosphytidylinositols such as soybean 1-α-phosphatidylinositol (PI), phosphatidic acids, diacetylphosphate, phosphatidylglycerols and diphosphatidylglycerols as well as sphingomyelins. Suitable synthetic saturated compounds such as dimyristoylphosphatidylcholine and dimyristoylphosphatidylglycerol or unsaturated species such as dioleoylphosphatidylcholine or dilinoleoylphospatidylcholine may also be used in the liposome formulations. Other synthetic phospholipids are phosphatidyl cholines, such as dipalmitoylphosphatidyl choline (DPPC), dimyristoyl phosphatidyl choline (DMPC), distearoyl phosphatidyl choline (DSPC), 1,2-di-o-hexadecyl-sn-glycero-3-phosphocholine (DHPC), and phosphatidyl glycerols such as dipalmitoyl glycerol (DPPG) and dimyristoyl phosphatidyl glycerol (DMPG). Other compounds lacking phosphorous, such as members of the glycolipids, and glycosphingolipid, ganglioside and cerebroside families, are also within the group designated as amphipathic lipids.

In another embodiment, the PEGylated compound comprises a hydrophilic compound that is derivatized with one or more PEG residues. Hydrophilic compounds are compounds that are readily soluble in water at room temperature. Hydrophilic compounds include amino acids, short chain alcohols and carboxylic acids, water soluble vitamins and derivatives, such as vitamin C and the like.

The present invention provides lipid-based formulations of the compounds of Formulae I, Ia, Ib, I-A, II, IIa or IIb to improve solubility and bioavailability. In one embodiment, the compound is formulated in combination with a PEGylated vitamin E analogue. In another embodiment, the vitamin E derivative is PEGylated D-alpha-tocopheryl poly(ethylene glycol) succinate. In various embodiments, the formulations comprise PEGylated vitamin E derivatives that comprise PEG residues of different size. For example, in some embodiments, TPGS modified with PEG residued of molecular weight from 100-40,000 are provided. In other embodiments, TPGS derivatized with PEG residues of molecular weight of 100-1000, 1000-40000, 1000-20000, 5000-40000, 10000-40000, 20000-40000, 500-1000, 1000-5000, 1000-20000, or 500-2000 are provided.

Preferably, the vitamin E derivative is D-alpha-tocopheryl poly(ethylene glycol) 1000 succinate (TPGS 1000, Eastman Chemical Company, Kingsport, Tenn.). TPGS 1000 is an FDA-approved, widely used form of vitamin E comprised of a hydrophilic polar (water-soluble) head and a lipophilic (water-insoluble) alkyl tail. TPGS 1000 has been used as a solubilizer, an emulsifier and as a vehicle for lipid-based drug delivery formulations. Most recently, TPGS 1000 has been recognized as an effective oral absorption enhancer. An enhancing effect is consistent with a surfactant-induced inhibition of P-glycoprotein (P-gp), and perhaps other drug transporter proteins. For example, the adsorption of highly lipophilic drug cyclosporine was found to be enhanced by combining it with TPGS (Sokol et al., The Lancet, 1991, 338, 212-215).

When mixed with aqueous TPGS, capuramycin analogues of Formula I, Ia, Ib, I-A, II, IIa or IIb are completely soluble and can be administered orally and parenterally. The soluble mixtures of capuramycin or capuramycin analogues exhibit improved intracellular activity compared to drug dissolved in dimethylsulfoxide (DMSO). This improved effect may be due to increased membrane permeability or blocking of P-gp mediated drug efflux.

In one embodiment, the TPGS formulation of the compound of Formulae I, Ia, Ib, I-A, II, IIa or IIb comprises a concentration of about 0.1% to about 15% (wt. %), TPGS in a pharmaceutically acceptable diluent. In another embodiment, the formulation comprises ≧0.5% by weight TPGS. In still other embodiments, the formulation comprises about 1% to about 10% TPGS, or about 1.5% to about 10% TPGS by weight. More preferably, the formulation comprises about 2% to about 10%, about 2% to about 5% or about 2% to about 3% TPGS by weight in a pharmaceutically acceptable diluent. Typically, the relative ratio of TPGS to the active compound will be about 10:1 to about 1:1 (wt./wt. ratio). In some embodiments, the relative ratio of TPGS to the compound will be about 5:1 to about 1:1 or about 3:1 to about 1:1.

In preferred embodiments, formulations comprising the capuramycin analogues SQ922, SQ641 or SQ997 with TPGS are provided.

### Insoluble Particulate Formulations

In another aspect of the invention, capuramycin analogues of Formulae I, Ia, Ib, I-A, II, IIa or IIb are provided in insoluble particulate formulations as described below. The novel particulate formulations exhibit surprisingly improved bioavailability and improved intracellular efficacy.

### TPGS Micelles

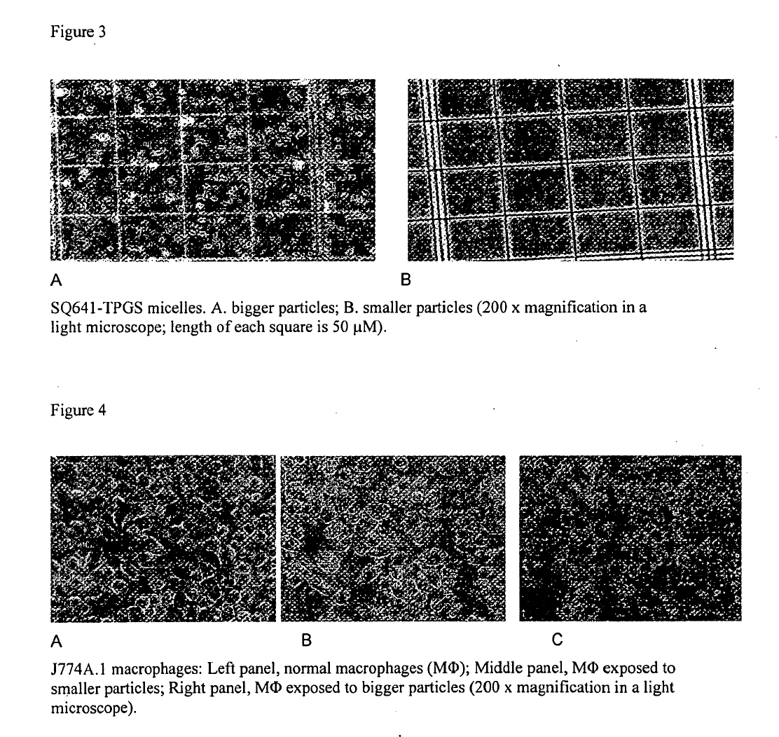

In one embodiment, micelles comprising a capuramycin analogue in combination with a PEGylated compound are provided. In another embodiment, capuramycin or capuramycin analogues of Formula I, Ia, Ib, I-A, II, IIa or IIb are mixed with TPGS to form micelles. Mixing the capuramycin analogues with TPGS with sonication forms stable rod shaped micelles without the need for an emulsifier. The particle size and drug loading of the micelles can be controlled by varying the intensity of the sonication, the pH, the molarity of the solution, the temperature and the volume of the suspension. FIG. 3 shows TPGS-SQ641 micelles of two different particle size distributions prepared by sonication of a 40% sucrose suspension of SQ641 and TPGS.

In one embodiment, the capuramycin—or capuramycin analogue-TPGS micelle formulation comprises a ratio of about less than or equal to 2:1 (wt.:wt.) TPGS to capuramycin or capuramycin analogue of Formulae I, Ia, Ib, I-A, II, IIa or IIb. More typically, the TPGS-micelle formulation comprises a ratio of about ≦1.5:1, TPGS to capuramycin or capuramycin analogue. In still another embodiment, the TPGS-micelle formulation comprises a ratio of ≦1:1, TPGS to capuramycin or capuramycin analogue.

In preferred embodiments, a TPGS micelle formulation comprising the capuramycin analogue SQ641 is provided.

The TPGS micelles comprising capuramycin or capuramycin analogues of Formulae I, Ia, Ib, I-A, II, IIa or IIb may be prepared by methods known in the art. In one embodiment of the invention, the TPGS-micelle formulations are prepared by mixing capuramycin or capuramycin analogue of Formulae I, Ia, Ib, I-A, II, IIa or IIb in TPGS diluted in a pharmaceutically acceptable diluent and energetically sonicating the mixture until a white suspension is formed. The resulting micelle suspension may be isolated by any suitable method, such as centrifugation, gel-permeation chromatography and filtration through a filter of known pore size, and evaluated for drug concentration. The micelles comprising the compounds of Formulae I, Ia, Ib, I-A, II, IIa or IIb and TPGS are very stable and can be dried into a powder and stored for later reconstitution into a suitable carrier without affecting the integrity of the micelles. In one embodiment, the diluent is water or a saline solution. In other embodiments, the diluent is a biologically acceptable buffer, including phosphate buffers, phosphate buffered saline and the like. In another embodiment, the diluent is a sucrose solution. Other suitable pharmaceutically acceptable diluents known to those skilled in the art may be used. The uptake of the micelles by macrophages was evaluated by isolating pellets formed from small 50 μL samples of micelle suspensions and incubating different concentrations of small-particle and large particle suspensions with J774A.1 macrophages for 24 hours. FIG. 4 shows J774A.1 macrophages that have been exposed to smaller micelle particles and larger micelle particles. It was found that TPGS micelles comprising the capuramycin analogue SQ641 are quickly and efficiently ingested by macrophages, resulting in a 5-10 fold increase in activity against Mycobacterium tuberculosis, compared with formulations of the drug without TPGS, such as in aqueous carriers or in solvents such as DMSO.

### Liposome Formulations

In another novel and inventive aspect of the invention, liposome formulations of capuramycin or capuramycin analogues of Formulae I, Ia and II exhibit improved bioavailability and improved intracellular activity compared with standard formulations of the drugs. Importantly, the novel formulations also exhibit decreased P-gp mediated drug efflux of the capuramycin analogues. In another embodiment, the present invention provides a liposome formulation of capuramycin or capuramycin analogues of Formulae I, Ia, Ib, I-A, II, IIa or IIb, wherein the active compounds are encapsulated in a liposome. Liposomes are self-assembling vesicles with an inner compartment surrounded by a lipid bilayer that commonly includes phospholipids and cholesterol. Both hydrophilic and hydrophobic drugs can be effectively encapsulated into liposomes. The pharmacokinetic profile of the drug is determined by the physicochemical properties of the liposomes. Liposomes typically have low toxicity and protect the drug from degradation. Liposome formulations of drugs are known and there are a number of FDA-approved liposome-encapsulated formulations. For example Ambisome® is a liposomal preparation of amphotericin B and Doxil®, a liposomal formulation of doxorubicin, are both approved for intravenous administration. Many liposomal antibiotics are superior to free antibiotics, whether the pathogen is located in the phagosome or in the cytoplasm (Salem et al., Methods Enzymol, 2005, 391, 261-291). For example, it has been shown that liposomal amikacin, streptomycin and ciprofloxacin are much more effective against intracellular M. avium than their free counterparts (Duzgunes et al., Antimicrob. Agents Chemother., 1988, 32(9), 1404-1411; J. Infect. Diseases, 1991, 164(1), 143-151; Antimicrob. Agents Chemother., 1996, 40(11), 2618-2621). Liposomal formulations of capuramycin and capuramycin analogues of Formulae I, Ia, Ib, I-A, II, IIa or IIb can be targeted to macrophages, a site of Mycobacterium replication in mammalian hosts.

A wide variety of liposomes are known and methods of forming liposomes are well known in the art. Any liposome formulation known in the art to combine with compounds that exhibit low aqueous solubility and methods of preparing these liposomes are embraced by the present invention. Liposome formulations for pharmaceutical applications can be made either by combining drug and lipid components before the formation of vesicles or by “loading” lipid vesicles with drug after they have formed. Liposome formulations of drugs are described in U.S. Pat. No. 5,154,930 to Popescu et al, U.S. Pat. No. 6,613,352 to Lagace et al., U.S. Pat. No. 4,522,803 to Lenk et al., U.S. Pat. No. 4,588,758 to Fountain et al., WO 93/23015 to Da Cruz et al., and WO 94/12155 to Proffitt et al., all of which are incorporated herein by reference in their entirety. Other liposome formulations in which the drug is loaded into a pre-formed vesicle are described in U.S. Pat. Nos. 5,785,987 and 5,800,833 to Hope et al., and U.S. Pat. No. 5,837,282 to Fenske et al., all of which are hereby incorporated by reference in their entirety. Furthermore, the inventive liposomes comprising a compound of Formulae I, Ia, Ib, I-A, II, IIa or IIb include, but are not limited to, unilamellar, multilamellar, plurilamellar liposomes or reverse phase evaporation vesicles.

Another class of liposomes is characterized as having substantially equal interlamellar solute distribution. This class of liposomes is denominated as stable plurilamellar vesicles (SPLV) as defined in U.S. Pat. No. 4,522,803 to Lenk et al., which is incorporated by reference in its entirety, includes monophasic vesicles as described in U.S. Pat. No. 4,588,578 to Fountain et al., which is incorporated by reference in its entirety, and frozen and thawed multilamellar vesicles (FATMLV) as described in “Solute Distributions and Trapping Efficiencies Observed in Freeze-Thawed Multilamellar Vesicles,” Mayer et al., Biochima et Biophysica Acta. 817:193-196 (1985).

The liposomes can be prepared by any of the techniques now known or subsequently developed for preparing liposomes. For example, the liposomes can be formed by the conventional technique for preparing multilamellar lipid vesicles (MLVs), that is, by depositing one or more selected lipids on the inside walls of a suitable vessel by dissolving the lipids in chloroform and then evaporating the chloroform, and by then adding the aqueous solution which is to be encapsulated to the vessel, allowing the aqueous solution to hydrate the lipid, and swirling or vortexing the resulting lipid suspension. This process engenders a mixture including the desired liposomes.

Alternatively, techniques used for producing large unilamellar lipid vesicles (LUVs), such as reverse-phase evaporation, infusion procedures, and detergent dilution, can be used to produce the liposomes. A review of these and other methods for producing lipid vesicles can be found in the text Liposome Technology, Volume I, Gregory Gregoriadis Ed., CRC Press, Boca Raton, Fla., (1984), which is incorporated herein by reference. For example, the lipid-containing particles can be in the form of steroidal lipid vesicles, stable plurilamellar lipid vesicles (SPLVs), monophasic vesicles (MPVs), or lipid matrix carriers (LMCs) of the types disclosed in Lenk, et al. U.S. Pat. No. 4,522,803, and Fountain, et al. U.S. Pat. Nos. 4,588,578 and 4,610,868, the disclosures of which are incorporated herein by reference. A particularly preferred method for preparing LUVs is described in U.S. Pat. No. 5,008,050.

Traditional methods of loading conventional drugs into liposomes include an encapsulation technique and a transmembrane potential loading method. In the encapsulation technique, the drug and liposome components are dissolved in an organic solvent or mixture of solvents in which all species are miscible, and then concentrated to a dry film. A buffer is then added to the dried film and liposomes are formed having the drug incorporated into the vesicle walls. In a modification of this encapsulation technique, the drug can be placed into a buffer and added to a dried film of only lipid components. In this manner, the drug will become encapsulated in the aqueous interior of the liposome. The buffer which is used in the formation of the liposomes can be any biologically compatible buffer solution of, for example, isotonic saline, phosphate buffered saline, or other low ionic strength buffers.

Another method of liposome formation is by the infusion of lipid solvent such as diethyl ether or ethanol which contains phospholipids into an aqueous solution containing a pharmacological agent resulting in the formation of liposomes which entrap a portion of the aqueous solution. This procedure cannot be used to entrap lipid soluble pharmacological agents soluble in fat or fat solvents due to the very limited solubility of such agents in an aqueous solution.

Alternative loading processes, termed “active loading,” involve the use of transmembrane potentials. Transmembrane potential loading has been described in detail in U.S. Pat. No. 4,885,172, U.S. Pat. No. 5,059,421, U.S. Pat. No. 5,171,578, U.S. Pat. No. 5,316,771 and U.S. Pat. No. 5,380,531, all of which are hereby incorporated by reference in their entirety. Briefly, the transmembrane potential loading method is used for a number of conventional drugs which can exist in a charged state when dissolved in an appropriate aqueous medium. Preferably, the drug will be relatively lipophilic so that it will partition into the liposome membranes. The loading process is carried out by creating a transmembrane potential across the bilayers of the liposomes. The transmembrane potential is generated by creating a concentration gradient for one or more charged species (e.g., Na⁺, K⁺, H⁺, NH₄⁺, RNH₄⁺, where R is an alkyl or aryl group, and the like) across the membranes. This concentration gradient is generated by producing liposomes having different internal and external media. Thus, for a drug which is negatively charged when ionized, a transmembrane potential is created across the membranes which has an inside potential which is positive relative to the outside potential. For a drug which is positively charged, the opposite transmembrane potential would be used.

A method of encapsulating a lipophilic active agent into a liposome by creating a different pH or salt environment across the lipid membrane is described in U.S. Pat. Nos. 5,785,987 and 5,800,833 to Hope et al., and U.S. Pat. No. 5,837,282 to Fenske et al., which are incorporated herein by reference in their entirety. Briefly, a liposome is formed that encapsulates an acidic or charged solution inside the vesicle. The external solution is exchanged for a neutral medium and the active compound is added to the liposomal suspension, creating a gradient across the membrane. The active compounds, which can be protonated are drawn into the acidic or charged internal liposome compartment.

Liposomes prepared in the method of the invention may be dehydrated for longer storage, if desired. In one embodiment, the lipid vesicles are loaded with the therapeutic agent, dehydrated for purposes of storage, shipping, and the like, and then rehydrated at the time of use. The liposomes are preferably dehydrated under reduced pressure using standard freeze-drying equipment or equivalent apparatus. The lipid vesicles and their surrounding medium can also be frozen in liquid nitrogen before being dehydrated or not, and placed under reduced pressure. Dehydration without prior freezing takes longer than dehydration with prior freezing, but the overall process is gentler without the freezing step, and thus there is subsequently less damage to the lipid vesicles and a smaller loss of the internal contents.

To ensure that the liposomes will survive the dehydration process without losing a substantial portion of their internal contents, one or more protective sugars may be used to interact with the lipid vesicle membranes and keep them intact as the water in the system is removed. A variety of sugars can be used, including such sugars as trehalose, maltose, sucrose, glucose, lactose, and dextran. In general, disaccharide sugars have been found to work better than monosaccharide sugars, with the disaccharide sugars trehalose and sucrose being most effective. Other more complicated sugars can also be used. For example, aminoglycosides, including streptomycin and dihydrostreptomycin, have been found to protect lipid vesicles during dehydration.

The amount of sugar to be used depends on the type of sugar used and the characteristics of the lipid vesicles to be protected. See, U.S. Pat. No. 4,880,635 and Harrigan, et al., Chem. Phys. Lipids 52:139-149 (1990), the disclosures of which are incorporated herein by reference. Persons skilled in the art can readily test various sugar types and concentrations to determine which combination works best for a particular lipid vesicle preparation.

Once the lipid vesicles have been dehydrated, they can be stored for extended periods of time until they are to be used. The appropriate temperature for storage will depend on the make up of the lipid vesicles and the temperature sensitivity of whatever materials have been encapsulated in the lipid vesicles. For example, as is known in the art, various pharmaceutical agents are heat labile, and thus dehydrated lipid vesicles containing such agents should be stored under refrigerated conditions so that the potency of the agent is not lost. Also, for such agents, the dehydration process is preferably carried out at reduced temperatures, rather than at room temperature.

In certain embodiments, it is desirable to target the liposomes of the invention using targeting moieties that are specific to a particular cell type, tissue, and the like. Targeting of liposomes using a variety of targeting moieties (e.g., ligands, receptors and monoclonal antibodies) has been previously described (see, e.g., U.S. Pat. Nos. 4,957,773 and 4,603,044, both of which are incorporated herein by reference).

The lipids used to prepare the liposome formulations are not limited and include a wide variety of lipids including, but are not limited to, saturated lipids, non-saturated lipids, synthetic lipids or lipids derived from natural sources. The lipids used to form the liposome formulations may be neutral, anionic, cationic or zwitterionic in nature. Lipids may include suitable phospholipids, phosphoglycerides, or other lipids include those obtained from soy, egg or plant sources or those that are partially or wholly synthetic. Phosphoglycerides include, but are not limited to, phosphatidylcholines, phosphatidylethanolamines, lysophosphatidylcholines, lysophosphatidylethanoloamines, phosphatidylserines, phosphytidylinositols such as soybean 1-α-phosphatidylinositol (PI), phosphatidic acids, diacetylphosphate, phosphatidylglycerols and diphosphatidylglycerols as well as sphingomyelins. Suitable synthetic saturated compounds such as dimyristoylphosphatidylcholine and dimyristoylphosphatidylglycerol or unsaturated species such as dioleoylphosphatidylcholine or dilinoleoylphospatidylcholine may also be used in the liposome formulations. Other synthetic phospholipids are phosphatidyl cholines, such as dipalmitoylphosphatidyl choline (DPPC), dimyristoyl phosphatidyl choline (DMPC), distearoyl phosphatidyl choline (DSPC), 1,2-di-o-hexadecyl-sn-glycero-3-phosphocholine (DHPC), and phosphatidyl glycerols such as dipalmitoyl glycerol (DPPG) and dimyristoyl phosphatidyl glycerol (DMPG). Other compounds lacking phosphorous, such as members of the glycolipids, and glycosphingolipid, ganglioside and cerebroside families, are also within the group designated as amphipathic lipids. Salts of acid derivatives of sterols and tocopherols such tocopherol hemisuccinate are also amphipathic and may be used in the formulations. Cholesterol, glycerides and triglycerides are also suitable lipid compounds. Ionic detergents such as octadecanylsulfonate are also included. In addition, mixtures of two or more lipids may be used in the liposome formulations.

During preparation of the liposomes organic solvents may be used to dissolve the lipids. Suitable solvents include, but are not limited to, chloroform, methanol, ethanol, dimethylsulphoxide (DMSO), methylene chloride, ether, acetone and solvent mixtures. Typically, the active compound is mixed with the lipid solution and the mixture is concentrated by evaporating the solvent or using other means, leaving a film. Other lipid solvents such as polyethylene glycol and polypropylene glycol may be used in the formulation. The film is then hydrated with an aqueous solvent such as aqueous glucose, sodium chloride, dextran, mannitol, dextrose, ringer acetate, sodium bicarbonate, HEPES-buffered saline, ammonium sulfate, phosphate buffer and the like. The pH of the internal environment of the liposomes may be adjusted with phosphate, acetate, or citrate buffers or other pharmaceutically acceptable buffers. In one embodiment, the pH of the solution may be adjusted to be slightly acidic.

In one embodiment, an aqueous solvent mixture comprising TPGS may be used to hydrate the film during the formation of the eliposomes. In addition to aqueous TPGS, the solvent mixture may comprise one or more co-solvents. In one embodiment a solvent mixture comprising water, TPGS and dimethylsulfoxide (DMSO) or the like may be used to hydrate the liposome film (see Duzgunes et al., Antimicrob. Agents Chemother. 1988, 32(9), 1404-11; J. Infect. Dis. 1991, 164(1), 143-45; and Majumdar et al., Antimicrob. Agents Chemother., 1992, 36(12), 2808-2815).

The liposomes may be sized by extrusion of the liposome through a small-pore filter. In one embodiment, the filter is a polycarbonate membrane or an asymmetric ceramic membrane (see, U.S. Pat. No. 5,008,050 and Hope, et al., in: Liposome Technology, vol. 1, 2d ed. (G. Gregoriadis, Ed.) CRC Press, pp. 123-139 (1992), the disclosures of which are incorporated herein by reference). Typically, the suspension is cycled through the membrane one or more times until the desired liposome size distribution is achieved. The liposomes may be extruded through successively smaller-pore membranes, to achieve a gradual reduction in liposome size. A wide range of filter pore diameters are available and the diameter used is dependent on the desired size distribution of the liposomes. In one embodiment, filters with a pore diameter of about 50 to 300 nm are used. In another embodiment, filters with a pore diameter of about 50 nm are used. In still another embodiment, a filter with a pore diameter of about 100 nm is used. Preferably, the filtration is done prior to removal of any unencapsulated compound or before exchanging the buffer since filtration may result in temporary disruption of liposomal membranes.

Other useful sizing methods such as sonication, solvent vaporization or reverse phase evaporation are known to those of skill in the art. One sizing method is described in U.S. Pat. No. 4,737,323, incorporated herein by reference. Sonicating a liposome suspension either by bath or probe sonication produces a progressive size reduction down to small unilamellar vesicles less than about 0.05 microns in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 0. 1 and 0.5 microns, are observed. The size distribution of the liposomes may be determined by known methods, such as dynamic light scattering.

The amount of capuramycin or capuramycin analogue encapsulated in the liposome may be determined by disrupting a known amount of the liposomal suspension and analyzing the resulting mixture for the concentration of the compound. The liposomal suspension may be disrupted by known methods, such as by mixing with a detergent (e.g. C₁₂E₈) or an alcohol solvent (e.g. 90% methanol). Analysis of the disrupted mixture may be accomplished by any known methods including, HPLC and the like.

In one embodiment, capuramycin or a capuramycin analogue of the invention is loaded into pre-formed liposomes which comprise a solvent medium inside the liposomes that has a different pH or composition than the solvent medium outside of the liposomes. Typically, the solvent medium inside the liposomes will have a lower pH than the solvent medium outside of the liposomes. In one embodiment, the solvent medium inside the liposomes will comprise a pharmaceutically acceptable buffer solution including, but not limited to, citrate, phosphate, ammonium sulfate, ammonium acetate, ammonium tartrate, trishydroxymethylaminomethane (TRIS) buffers, and the like. Also suitable are acetate, formate, sulfate, or tartrate buffers comprising cations such as magnesium, potassium, sodium, trimethylammonium and the like. In another embodiment, the medium inside the liposomes comprises a salt of an amine. The salt may be any pharmaceutically acceptable salt. The type of amine is not limited and can be varied based on the pKa value and the basicity of the active compound.

The liposomes which are used for loading the active agent using a transmembrane gradient may be formed from standard vesicle-forming lipids by a standard method known in the art. The selection of lipids is generally guided by consideration of, e.g., liposome size and stability of the liposomes in the bloodstream.

To create the concentration gradient, the original external medium is replaced by a new external medium having a different concentration of the charged species or a totally different charged species. The replacement of the external medium can be accomplished by various techniques, such as, by passing the lipid vesicle preparation through a gel filtration column, e.g., a Sephadex column, which has been equilibrated with the new medium, or by centrifugation, dialysis, or related techniques.

Depending upon the permeability of the lipid vesicle membranes, the full transmembrane potential corresponding to the concentration gradient will either form spontaneously or a permeability enhancing agent, e.g., a proton ionophore may have to be added to the bathing medium. If desired, the permeability enhancing agent can be removed from the preparation after loading has been completed using chromatography or other techniques. In either case, a transmembrane potential having a magnitude defined by the Nernst equation will appear across the lipid vesicles' membranes. The change in composition of the external phase causes an outflow of a neutral component from the interior encapsulated medium to the external medium as the active agent migrates inside the liposome. This outflow also results in a reverse pH gradient by accumulation of hydrogen ions left behind in the internal aqueous phase. An influx of a neutral form of a protonable therapeutic agent into the liposomes replaces the intra-liposome medium species.

In one embodiment, liposomes are formed that encapsulate a citrate or ammonium sulfate buffer at a pH of about 4. The medium outside of the liposomes may be exchanged with a neutral medium by standard methods and then incubated with the active compound to form the liposomes. In one embodiment, the neutral medium is a neutral buffer such as NaCl or NaCl/HEPES (4-(2-hydroxyethyl)-1-piperazineethanesulfonic acid), at a pH of about 7.4. In another embodiment, capuramycin or a capuramycin analogue is dissolved in a mixture of the neutral medium and a suitable organic solvent, such as methanol, ethanol, DMSO and the like, and incubated with the pre-formed liposomes to form the liposome formulation.

In another embodiment, the liposome formulations comprise sterically stabilized liposomes (see Gangadharam et al., Antimicrob. Agents and Chemother., 1995, 39(3), 725-730). These liposomes have been found to exhibit prolonged circulation times in the bloodstream and localize in infected lung tissue rather than normal tissue. In various embodiments, the sterically stabilized liposomes comprise polyethyleneglycol-distearoyl phosphatidylethanolamine (PEG-DSPE), distearoylphosphatidylcholine (DSPC), soybean phosphatidylinositol, cholesterol or a combination thereof. In a preferred embodiment, the sterically stabilized liposome comprises a mixture of polyethyleneglycol-distearoyl phosphatidylethanolamine (PEG-DSPE), distearoylphosphatidylcholine (DSPC) and cholesterol. In a particular embodiment, the sterically stabilized liposome comprises PEG-DSPE:DSPC:cholesterol in a molar ratio of about 1:9:7. In still another embodiment the sterically stabilized liposome comprises PEG-DSPE:DSPC:cholesterol in a molar ratio of about 1:9:6.7.

In another embodiment, the sterically stabilized liposome comprises a mixture of soybean phosphatidylinositol, DSPC and cholesterol. In a particular embodiment, the sterically stabilized liposome comprises soybean phosphatidylinositol, DSPC and cholesterol in a molar ratio of about 1:9:7 or in a molar ratio of about 1:9:6.7.

In another embodiment, the liposomes comprise egg phosphatidylglycerol, egg phosphatidylcholine, cholesterol or a mixture thereof. In a particular embodiment, the liposome comprises a mixture of egg phosphatidylglycerol, egg phosphatidylcholine, cholesterol. In another embodiment, the liposome comprises a mixture of egg phosphatidylglycerol, egg phosphatidylcholine, cholesterol in a molar ratio of about 1:9:7, respectively. a mixture of egg phosphatidylglycerol, egg phosphatidylcholine, cholesterol in a molar ratio of about 1:9:6.7, respectively.

In preferred embodiments, the invention provides liposome formulations of the capuramycin analogues SQ922, SQ641 and SQ997.

The liposomes of the present invention can be administered alone but will generally be administered together with a pharmaceutically acceptable carrier. The preparations may be administered orally or parenterally, or intravenously.

### Nanoparticle Formulations

In another aspect of the invention, formulations comprising a capuramycin analogue of the invention and nanoparticle carriers are provided that exhibit improved bioavailability, sustained release profiles and decreased P-gp mediated drug efflux compared with standard formulations of the drugs. The inventive nanoparticle formulations of the present invention provide remarkably improved drug delivery of capuramycin analogues compared with standard formulations of the drugs. The use of nanoparticles for the delivery of drugs has been investigated and provides advantages over standard drug delivery systems. For example, nanoparticle drug carriers offer the advantages of high stability and long shelf life, high carrier capacity, the ability to administer the drug by variable routes of administration, including oral, intravenous and inhalation administration, and the ability to formulate drugs for controlled release over time from the nanoparticle matrix. Importantly, nanoparticle carriers allow the incorporation of both hydrophilic and hydrophobic drugs. The anti-tuberculosis drugs rifampin, isoniazid and pyrazinamide have been co-encapsulated in poly(lactide-co-glycolide nanoparticles and administered orally to mice (see Pandey et al., Tuberculosis (Edinb), 2003, 83, 373-378). The drugs could be detected in circulation for 4 days (rifampin) and 9 days (isoniazid and pyrazinamide) and therapeutic concentrations were maintained in tissues for 9-11 days. In contrast, free drugs were cleared from the plasma within 12 to 24 hours.

The nanoparticle formulations of the invention include monolithic nanoparticles (nanospheres) in which the compound is adsorbed, dissolved or dispersed throughout the nanoparticle matrix as well as nanocapsules, in which the compound is confined to an aqueous or non-aqueous core surrounded by a shell-like wall. In another embodiment, the nanoparticles formulations comprise nanoparticles in which the compound is covalently attached to the matrix.

The nanoparticles are prepared from biocompatible or biodegradable polymers or co-polymers, including synthetic or natural polymers and co-polymers. The nanoparticles may be formed with graft, block or random co-polymers. In one embodiment, the nanoparticles are amphiphilic copolymers. Amphiphilic copolymers are comprised of sub-units or monomers that have different hydrophilic and hydrophobic characteristics. Typically, these sub-units are present in groups of at least two, comprising a block of a given character, such as a hydrophobic or hydrophilic block.

Methods for the formation of nanoparticles are known, and any suitable method may be used to prepare the inventive formulations. For example, U.S. Patent Application Publication No. US2004/0091546 to Johnson et al., which is hereby incorporated herein by reference in its entirety, describes a process for the preparation of nanoparticles of amphiphilic copolymers by flash precipitation which may incorporate an active agent.

Preferably the nanoparticle compositions have an average size less than 1000 nm and more preferably less than about 700 nm, less than about 500, less than about 400, less than about 200, less than about 100, less than about 40 nm. The average size is on a weight basis and is measured by light scattering, microscopy, or other appropriate methods. Preferably at least 65% of the particles by weight have a particles size less than 1000 nm, and more preferably at least 80% of the particles are less than 1000 nm, and even more preferable at least 95% of the particles on a weight basis have a particle size less than 1000 nm as measured by light scattering, microscopy, or other appropriate methods. In another embodiment, the nanoparticle compositions comprise microspheres wherein the particle size of the particles is greater than or equal to about 1000 nm for certain modes of administration which are suitable for larger particle size formulations of the drugs, such as oral administration.

Preferably, block copolymers include blocks substantially comprising the monomers of polystyrene, polyethylene, polybutyl acrylate, polybutyl methacrylate, polylactic acid, polyacrylic acid, polyoxyethylene or those that are biocompatible. Additional preferable polymers shown to be mucoadherents and preferable for incorporation into amphiphilic copolymers include, but are not limed to, monomers of poly (acrylic acid), poly(d-glucosamine), poly(d-glucaronic acid-N-acetylglucosamine), poly(N-isopropylacrylamide), poly(vinyl amine), and poly(methacrylic acid).

In graft copolymers, the length of a grafted moiety can vary. In addition, the grafting of the polymer backbone can be useful to enhance solvation or nanoparticle stabilization properties. For example, a grafted alkyl group on the hydrophobic backbone of a diblock copolymer of a polyethylene and polyethylene glycol should increases the solubility of the polyethylene block. Suitable chemical moieties grafted to the block unit of the copolymer include, but are not limited to, alkyl chains containing species such as, but not limited, to amides, imides, phenyl, carboxy, aldehyde or alcohol groups.

The amphiphilic copolymer can be selected from several groups of copolymers including polystyrenes, polyethyleneglycols, polyglutamic acids, hyaluronic acids, polyvinylpyrrolidones, polylysines, polyarginines, alginic acids, polylactides, polyethyleneimines, polyionenes, polyacrylic acids, and polyiminocarboxylates. Any biocompatible amphiphilic copolymer can be used. Preferably, the amphiphilic copolymer is comprised of diblock or triblock compositions containing at least one of the following: a polystyrene block, a polyethylene oxide block, a polybutylacrylate, a polyacrylic acid, polybutylmethacrylate block, or a polyethyleneoxide block.

In other embodiments, the biocompatible polymer or copolymer includes polyamides including polymers based on caprolactam monomers, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methylmethacryl ate), poly(ethylmethacrylate), poly(butylmethacrylate), poly(isobutylmethacrylate), poly(hexlmethacrylate), poly(isodecylmethacrylate), poly(laurylmethacrylate), poly(phenylmethacrylate), poly(methacrylate), poly(isopropacrylate), poly(isobutacrylate), poly(octadecacrylate), polyethylene, polypropylene poly(ethylene glycol), poly(ethylene oxide), poly(ethylene terephthalate), poly(vinyl alcohols), poly(vinyl acetate), poly vinyl chloride, polystyrene, polyhyaluronic acids, casein, gelatin, gluten, polyanhydrides, polyacrylic acid, alginate, chitosan, any copolymers thereof, and any combination of any of these.

In a preferred embodiment, capuramycin or a capuramycin analogue of Formulae I, Ia, Ib, I-A, II, IIa or IIb is co-precipitated with the amphiphilic copolymer to form a nanoparticle that incorporates the active compound. The target molecule should be substantially insoluble in solution created after the mixing process is complete.

In another preferred embodiment, nanoparticle formulations comprising a capuramycin analogue, including the capuramycin analogues SQ922, SQ641 and SQ997, are provided.

In another embodiment, nanoparticle formulations comprising a capuramycin analogue, including SQ922, SQ641 and SQ997, in combination with another active agent are provided

### Methods of Treatment

The present invention provides methods for the treatment or prophylaxis of a disease caused by a microorganism comprising administering an effective amount of capuramycin or a capuramycin analogue, including derivatized capuramycin or capuramycin analogues, such as an amino acid derivative or a PEGylated derivative of capuramycin or a capuramycin analogue of Formulae I, Ia, Ib, I-A, II, IIa or IIb to a host in need thereof. In a preferred embodiment, the host is a human patient.

In another embodiment, the present invention provides methods for the treatment or prophylaxis of a disease caused by a microorganism comprising administering an effective amount of a formulation comprising capuramycin or a capuramycin analogue of Formulae I, Ia, Ib, I-A, II, IIa or IIb, and a vitamin E derivative, such as TPGS.

In another embodiment, the invention provides methods for the treatment or prophylaxis of a disease caused by a microorganism comprising administering an effective amount of a liposome formulation of capuramycin or a capuramycin analogue of Formulae I, Ia, Ib, I-A, II, IIa or IIb or a nanoparticle formulation comprising capuramycin or a capuramycin analogue of Formulae I, Ia, Ib, I-A, II, IIa or IIb. In one embodiment, the invention provides a method for the treatment of a disease caused by the microorganism, M. tuberculosis. In another embodiment, a method for the treatment of a mycobacterial disease is provided. In another embodiment, a method for the treatment of a non-tuberculosis mycobacterial infection is provided. The methods and compositions of the present invention are intended for the treatment of mycobacteria infections in human, as well as other animals. For example, the present invention may be particularly useful for the treatment of cows infected by M. bovis.

As used herein, the term “tuberculosis” comprises disease states usually associated with infections caused by mycobacteria species comprising M. tuberculosis complex. The term “tuberculosis” is also associated with mycobacterial infections caused by mycobacteria other than M. tuberculosis (MOTT). Other mycobacterial species include M. avium-intracellulare, M. kansarii, M. fortuitum, M. chelonae, M. leprae, M. africanum, and M. microti, M. avium paratuberculosis, M. intracellulare, M. scrofulaceum, M. xenopi, M. marinum, M. ulcerans.

In one embodiment, a method for the treatment of M. avium-intracellulare, M. kansarii, M. fortuitum, M. chelonae, M. leprae, M. africanum, and M. microti, M. avium paratuberculosis, M. intracellulare, M. scrofulaceum, M. xenopi, M. marinum, or M. ulcerans with capuramycin analogues of the invention is provided.

The present invention further comprises methods and compositions effective for the treatment of infectious disease, including but not limited to those caused by bacterial, mycological, parasitic, and viral agents. Examples of such infectious agents include the following: staphylococcus, streptococcaceae, neisseriaaceae, enterobacteriaceae, pseudomonadaceae, vibrionaceae, campylobacter, pasteurellaceae, bordetella, francisella, brucella, legionellaceae, bacteroidaceae, gram-negative bacilli, clostridium, corynebacterium, propionibacterium, gram-positive bacilli, anthrax, actinomyces, nocardia, mycobacterium, Helicobacter pylori, Streptococcus pneumoniae, Candida albicans, treponema, borrelia, leptospira, mycoplasma, ureaplasma, rickettsia, chlamydiae, systemic mycoses, opportunistic mycoses, protozoa, nematodes, trematodes, cestodes, adenoviruses, herpesviruses, poxviruses, papovaviruses, hepatitis viruses, orthomyxoviruses, paramyxoviruses, coronaviruses, picornaviruses, reoviruses, togaviruses, flaviviruses, bunyaviridae, rhabdoviruses, human immunodeficiency virus and retroviruses.

The present invention further provides methods and compositions useful for the treatment of infectious disease, including by not limited to, tuberculosis, leprosy, Crohn's Disease, acquired immunodeficiency syndrome, lyme disease, cat-scratch disease, Rocky Mountain Spotted Fever and influenza.

As discussed herein, there is a need in the art for novel compounds and methods that are effective against infectious disease. More particularly, there is a need for novel compounds and methods for the effective treatment of Mycobacterial disease. The instant invention satisfies the long felt need of the prior art by providing novel compositions and methods that are effective in the treatment of infectious disease, including but not limited to, tuberculosis.

### Combination Treatment

It has been recognized that drug-resistant variants of mycobacteria exist which complicates the effective treatment of the disease with standard anti-tuberculosis agents. The efficacy of a drug against a tuberculosis infection can be prolonged, augmented, or restored by administering the compound in combination or alternation with a second, and perhaps third, anti-tuberculosis drug. Alternatively, the pharmacokinetics, biodistribution or other parameter of the drug can be altered by such combination or alternation therapy.

The bactericidal activity of streptomycin, isoniazid, rifampin, ethambutol, and pyrazinamide alone and in combination against Mycobacterium tuberculosis is discussed by Dickinson et al. (Am Rev Respir Dis 116(4): 627-35): Log-phase cultures of Mycobacterium tuberculosis in Tween-albumin medium were exposed to streptomycin, isoniazid, rifampin, ethambutol, and pyrazinamide in concentrations in the range likely to be present in serum during treatment of patients. The bactericidal activity of the drugs was measured as the decrease in viable counts at 4 and 7 days. The activity of single drugs was highest for streptomycin and next highest for rifampin and isoniazid, but ethambutol only started to kill after 4 days. When exposed to 2 drugs, bactericidal synergism was found with streptomycin/isoniazid and isoniazid/ethambutol; additivity with streptomycin/rifampin; indifference, with isoniazid rifampin and streptomycin/ethambutol; and antagonism, with rifampin/ethambutol and isoniazid/pyrazinamide. When cultures were exposed to the 3 drugs, isoniazid, rifampin, and ethambutol, marked antagonism was found between isoniazid and rifampin, whereas the addition of isoniazid or an increase in its concentration increased the bactericidal activity.

In one embodiment of the present invention, capuramycin or capuramycin analogues of Formulae I, Ia, Ib, I-A, II, IIa or IIb may be administered in combination or alternation with one or more other active agents for the treatment of a mycobacterial disease. In combination therapy, an effective dosage of two or more agents is administered together, whereas during alternation therapy an effective dosage of each agent is administered serially. The dosages will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

It has been surprisingly found that administration of the capuramycin analogue SQ641 with other anti-tuberculosis drugs, such as ethambutol, isoniazid, streptomycin and the substituted ethylenediamine compound SQ109 (shown below), exhibits a synergistic efficacy against mycobacterial agents.

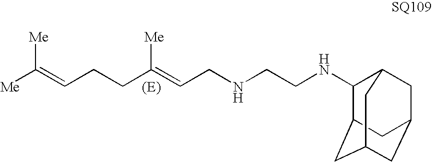

In one embodiment of the invention, capuramycin analogues are administered with one or more other antituberculosis drugs, including ethambutol, rifampicin, isoniazid, pyrazinamide, moxifloxacin, streptomycin, clarithromycin, amikacin, or SQ109. In another embodiment, administration of a capuramycin analogue in combination with one or more other antituberculosis drugs, including ethambutol, rifampicin, isoniazid, pyrazinamide, moxifloxacin, streptomycin, clarithromycin, amikacin, or SQ109, exhibits a synergistic efficacy against mycobacterial agents compared to administration of an equivalent dose of either compound alone.

In one embodiment, the capuramycin or a capuramycin analogue of Formulae I, Ia, Ib, I-A, II, IIa or IIb may be administered with one or more anti-tuberculosis drugs. In another embodiment, the invention provides pharmaceutical compositions comprising a capuramycin or a capuramycin analogue of Formulae I, Ia, Ib, I-A, II, IIa or IIb in combination with one or more active agents, such as another anti-tuberculosis drug, and a pharmaceutically acceptable carrier. In still another embodiment, methods for the treatment or prevention of a mycobacterial disease comprising administering capuramycin or a capuramycin analogue of Formulae I, Ia, Ib, I-A, II, IIa or IIb in combination with another active agent are provided.

In a further embodiment, a composition comprising capuramycin or a capuramycin analogue Formulae I, I-A, Ia, Ib, II, IIa or IIb in combination with one or more anti-tuberculosis drugs is provided that exhibits a synergistic efficacy against a mycobacterial disease, compared to an equivalent dosage of either drug alone.

In one embodiment, capuramycin or a capuramycin analogue of Formulae I, I-A, Ia, Ib, II, IIa or IIb is administered in combination with a substituted ethylenediamine compound of Formula III

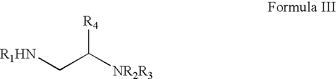

wherein R₁, R₂, and R₃ are independently H, alkyl; aryl; alkenyl; alkynyl; aralkyl; aralkenyl; aralkynyl; cycloalkyl; cycloalkenyl; heteroalkyl; heteroaryl; halide; alkoxy; aryloxy; alkylthio; arylthio; silyl; siloxy; a disulfide group; a urea group; amino; and the like; and R₄ is H, alkyl or aryl, alkenyl, alkynyl, aralkyl, aralkenyl, aralkynyl, cycloalkyl, cycloalkenyl.

In a particular embodiment, capuramycin or a capuramycin analogue of Formulae I, Ia, Ib, I-A, II, IIa or IIb may be administered with the ethylenediamine compound SQ109.

In another embodiment, capuramycin or a capuramycin analogue of Formulae I, I-A, Ia, Ib, II, IIa or IIb is administered in combination with one or more of ethambutol, rifampicin, isoniazid, pyrazinamide, moxifloxacin, streptomycin, clarithromycin, amikacin, SQ109, a compound of Formula III, or a combination thereof.

In a preferred embodiment, the capuramycin analogues SQ641, SQ922 or SQ997 are administered with one or more of ethambutol, rifampicin, isoniazid, pyrazinamide, moxifloxacin, streptomycin, clarithromycin, amikacin, SQ109, a compound of Formula III, or a combination thereof.

In another embodiment, a pharmaceutical composition comprising SQ641, SQ922 or SQ997 in combination with ethambutol, rifampicin, isoniazid, pyrazinamide, moxifloxacin, streptomycin, clarithromycin, amikacin, SQ109, a compound of Formula III, or a combination thereof, and a pharmaceutically acceptable carrier is provided.

In another embodiment, the present invention comprises a composition effective against Mycobacterium-fortuitum, Mycobacterium marinum, Helicobacter pylori, Streptococcus pneumoniae and Candida albicans comprising capuramycin or a capuramycin analogue of Formulae I, Ia, Ib, I-A, II, IIa or IIb, including SQ641, SQ922 or SQ997, in combination with one or more antitubercular agents wherein the antitubercular agents, include but are not limited to ethambutol, rifampicin, isoniazid, pyrazinamide, moxifloxacin, streptomycin, clarithromycin, amikacin, SQ109, and analogues thereof.

Therapeutics, including compositions comprising the capuramycin or capuramycin analogues of Formulae I, Ia, Ib, I-A, II, IIa or IIb and combinations of these compound with other active agents can be prepared in physiologically acceptable formulations, such as in pharmaceutically acceptable carriers, using known techniques. For example, the active agent is combined with a pharmaceutically acceptable excipient to form a therapeutic composition.

### Pharmaceutical Carriers and Form of Administration

Pharmaceutically acceptable carriers that may be used in these pharmaceutical compositions are generally known in the art. They include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, solvents, salts or electrolytes, such as protamine sulfate, disodium hydrogen phosphate, potassium hydrogen phosphate, sodium chloride, zinc salts, silicates, colloidal silica, magnesium trisilicate, polyvinyl pyrrolidone, cellulose-based substances, polyethylene glycol, sodium carboxymethylcellulose, polyacrylates, waxes, oils, carbohydrate polymers, polyethylene-polyoxypropylene-block polymers, polyethylene glycol and wool fat. Pharmaceutically accepted vehicles can contain mixtures of more than one excipient in which the components and the ratios can be selected to optimize desired characteristics of the formulation including but not limited to shelf-life, stability, drug load, site of delivery, dissolution rate, self-emulsification, control of release rate and site of release, and metabolism.

The compositions of the present invention may be administered orally, parenterally, by inhalation, topically, rectally, nasally, buccally, vaginally, transdermally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, sub-cutaneously, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other surface-active emulsifying agents or bioavailability enhancers which are commonly used in the manufacture of pharmaceutically acceptable solid, liquid, or other dosage forms may also be used for the purposes of formulation.

The pharmaceutical compositions of this invention may be prepared by techniques known in the art and may be orally administered in any orally acceptable dosage form including, but not limited to, capsules, tablets, pills, aqueous suspensions or solutions. In the case of tablets for oral use, carriers commonly used include but are not limited to celluloses, lactose, or corn starch. Lubricating agents, such as magnesium stearate, are also typically added. For oral administration in a capsule form, useful diluents or carriers include lactose and dried cornstarch. When aqueous suspensions or solutions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared using techniques known in the art including for example by mixing the agent with a suitable non-irritating excipient, which is solid at room temperature but liquid at rectal temperature, and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, the airways, or the lower intestinal tract. Suitable topical formulations are readily prepared for each of these areas or organs using techniques known in the art. For example, topical application for the lower intestinal tract can be effected in a rectal suppository formulation (see above) or in a suitable enema formulation. Topically-transdermal patches may also be used.

Implantable dosage units may be administered locally, for example, in the lungs, or may be implanted for systematic release of the therapeutic composition, for example, subcutaneously.

For topical or transdermal applications, the pharmaceutical compositions may be formulated by techniques known in the art in a suitable ointment or base containing the active component suspended or dissolved in one or more carriers. Carriers for topical administration of the compounds of this invention are well known in the art and include, but are not limited to, mineral oil, liquid petrolatum, white petrolatum, propylene glycol, polyoxyethylene, polyoxypropylene compound, emulsifying wax, and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

The pharmaceutical compositions of this invention may also be administered by nasal aerosol or inhalation. Examples of aerosol formulations include inhaler formulations for administration to the lungs. Such compositions are prepared according to techniques well-known in the art of pharmaceutical formulation and may be prepared as suspensions or solutions in saline, optionally employing benzyl alcohol or other suitable preservatives, absorption promoters to enhance bioavailability, fluorocarbons, and/or other conventional solubilizing or dispersing agents.

A sustained release matrix, as used herein, is a matrix made of materials, usually polymers, which are degradable by enzymatic or acid/base hydrolysis, or by dissolution. Once inserted into the body, the matrix is acted upon by enzymes and body fluids. The sustained release matrix is chosen desirably from biocompatible materials, including, but not limited to, liposomes, polylactides, polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide.

The dosage of the composition will depend on the condition being treated, the particular composition used, and other clinical factors, such as weight and condition of the patient, and the route of administration. A suitable dosage may range from 100 to 0.1 mg/kg. A more preferred dosage may range from 50 to 0.2 mg/kg. A more preferred dosage may range from 25 to 0.5 mg/kg. Tablets or other forms of media may contain from 1 to 1000 mg of capuramycin or a capuramycin analogue of Formulae I, I-A, Ia, Ib, II, IIa or IIb. Dosage ranges and schedules of administration similar to ethambutol or other anti-tuberculosis drugs may be used.

The composition may be administered in combination with other compositions and procedures for the treatment of other disorders occurring in combination with mycobacterial disease. For example, tuberculosis frequently occurs as a secondary complication associated with acquired immunodeficiency syndrome (AIDS). Patients undergoing AIDS treatment, which includes procedures such as surgery, radiation or chemotherapy, may benefit from the therapeutic methods and compositions described herein.

### EXAMPLES

The following examples are presented to aid in an understanding of the present invention and are not intended to, and should not be construed to, limit the invention in any way. It will be appreciated that other examples, alternatives, modifications and equivalents, including minor variations in chemical procedures, will be apparent to those skilled in the art, and that the invention is not limited to these specific illustrated examples.

### Example 1

### Anti mycobacterial Activity of Capuramycin Analogues

The in vitro activities of eleven chemically-modified capuramycin (CM) analogues against both fast- and slow-growing Mycobacteria was investigated: a laboratory strain of Mycobacterium smegmatis (MSMG), a laboratory strain and clinical isolates of Mycobacterium tuberculosis MTB, clinical isolates of M. avium complex (MAC), M. kansasii (MKN), and M. abscessus (MAB). In vitro tests included minimal inhibitory concentration (MIC), minimum bactericidal concentration (MBC), MIC/MBC ratio, Time-to-Kill, Post-antibiotic Effect (PAE), and assessment of synergy with front-line TB drugs. The MBC/MIC ratio was 1.0 for the capuramycin analogues SQ641, SQ922 and SQ997, suggesting that they are bactericidal antibiotics. All three compounds killed MTB much faster than other anti-TB drugs, with 90% killed within 48 hr compared to 5-7 days for other active drugs. The PAE of SQ641 against MTB (H37Rv) was 55 hr compared to 17 hr for INH at a similar concentration. SQ641, SQ922 and SQ997 caused rapid bacterial disintegration, resulting in complete clearance of MTB cultures. SQ641 also showed synergistic activity with EMB, the new diamine antitubercular SQ109, and streptomycin against MTB; with EMB and INH against MSMG; and with EMB against MAC. In potency for all in vitro tests and against all mycobacterial species, the TL1 inhibitors ranked in the following order: SQ641>SQ922>SQ997, with MIC and MBC ranging from 0.125 to 32.0 μg/ml, depending on mycobacterial species and isolate. Among the three chemically-modified analogues, SQ641 showed the best in vitro activity against the different Mycobacteria species.

Determination of MIC. MIC of drugs for laboratory and clinical strains of Mycobacterium tuberculosis (MTB) was determined by BACTEC radiometric method as described by Heifets (1991), “Antituberculosis Drugs: Antimicrobial Activity in vitro, Drug Susceptibility in the Chemotherapy of Microbacterial Infections, Boca Raton, Fla., CRC Press and Luna-Herrera et al., Antimicrob. Agents and Chemother. 1995, 39(2), 440-444. BACTEC vials were incubated at 37° C. and read daily for growth index (GI) in BACTEC TB-460 reader (Becton Dickinson, Towson, Md.) until the GI in 1/100 control reached ≧30 and showed an increase of at least 10 for 3 consecutive days. The minimal inhibitory concentration (MIC) was defined as the lowest concentrations of drug that caused GI equal to or less than that of the 1/100 dilution growth control. Capuramycin analogues SQ641, SQ922 and SQ997 were tested at concentrations ranging from 16 mg/mL-0.05 mg/mL. SQ641 and SQ922 were found to be better than SQ997 at killing drug-susceptible MTB, laboratory strain or clinical isolate (see Table 4 below).

TABLE 4
CM analog MIC for MTB

| | MIC μg/ml | | |
|---|---|---|---|
| Strain | SQ997 | SQ922 | SQ641 |
| H37Rv | 8 | 2 | 1 |
| 16 | 16 | 8 | 4 |
| 113 | 16 | 2 | 1 |
| 158 | 8 | 2 | 4 |
| 606 | 16 | 4 | 4 |
| 847 | 16 | 4 | 4 |
| 1351 | 16 | 2 | 1 |
| 1407 | 16 | 2 | 2 |
| 1498 | 16 | 4 | 4 |
| 1665 | 16 | 4 | 2 |
| 1703 | 16 | 8 | 4 |
| 1760 | >16 | 8 | 8 |
| 1782 | >16 | 8 | 2 |
| 1901 | 16 | 4 | 4 |
| 1921 | 16 | 4 | 4 |
| 2224 | 16 | 4 | 4 |
| 2554 | 16 | 4 | 4 |
| 2693 | 16 | 4 | 8 |
| 8266 | 8 | 2 | 2 |
| 8642 | 16 | 2 | 4 |

The effectiveness of the various capuramycin analogues of Table 3 against the non-tuberculosis mycobacteria (NTM) species M. avium complex (MAC), M. kansaii (MKN; ATCC12478 & 35775), M. abscessus (MAB; ATCC 700869), and M. smegmatis (MSMG; MC2155) was determined. Table 5 below shows the MIC of the capuramycin analogues against these infective agents. The MIC was determined by microdilution method in round bottomed 96-well microtiter plates (Wallace et al. J. Clin. Microbiol., 1986, 24(6), 9756-981). Drugs were diluted serially in 7H9 broth in 100 μl volumes. An equal volume of 1/100 dilution of mycobacterial cultures in 7H9 broth (adjusted to 0.1 λ₆₀₀) was dispensed into each well. Final drug concentrations ranged from 32 μg/ml-0.03 μg/ml. The last well in each row served as a drug-free growth control. Plates were placed in zip-lock bags and incubated at 37° C. The lowest concentration of drug with no visible growth at the end of 72 hr (MSMG) or 2 wk (MAC) was considered the MIC.

The MIC of capuramycin analogues SQ997, SQ922 and SQ641 against MSMG were 8.0, 2.0 and 2.0 μg/ml, respectively, whereas the MIC ranged from 1.0 μg/ml (SQ641)—>16.0 μg/ml (SQ997) for MTB (Table 4). The MIC₉₀ against MTB showed one dilution difference between the three analogues (16.0, 8.0 and 4.0 μg/ml, for SQ997, SQ922 and SQ641, respectively). Like the activity against MTB, the order of activity of the three compounds against MAC (Table 5) and MSMG was SQ641>SQ922>SQ997.

TABLE 5
Susceptibilities of Non-Tuberculous Mycobacteria (NTM)

| | MIC (μg/ml) | | | | |
|---|---|---|---|---|---|
| | MSMG | MAB | MKN | MKN | MAC |
| Compound | MC²155 | 700869 | ATCC35775 | ATCC12478 | [MIC90] |
| SQ997 | 8 | 16 | 4 | 16 | 4-32 [16] |
| SQ922 | 2 | 16 | 1 | 16 | 0.5->32 [16] |
| SQ641 | 2 | 0.5 | 0.25 | 1 | 0.125-2 [2] |
| RKS 2186 | 4 | 16 | 4 | >32 | 8-1 [8] |
| RKS 2033 | 4 | 2 | 2 | 16 | 1-8 [4] |
| RKS 2137 | 4 | 8 | 2 | >32 | 0.5-16 [8] |
| RKS 2243 | 4 | >32 | 2 | 32 | 0.5-8 [4] |
| RKS 2235 | 4 | 16 | 2 | >32 | 1-8 [4] |
| RKS 2244 | 1 | 4 | 2 | >32 | 0.5-8 [4] |
| RKS 2241 | 4 | 2 | 2 | 32 | 0.5-16 [8] |
| RS 120029 | >32 | >32 | >32 | >32 | 32->32 [32] |
| INH | 8 | >32 | 1 | 2 | ND |
| RIF | 8 | >32 | 0.125 | 0.125 | 0.062-4 [1] |
| SM | 0.5 | 8 | 2 | >32 | ND |
| EMB | 0.5 | >32 | 1 | 2 | 0.5-32 [16] |
| CLA | 2 | >32 | 0.25 | 0.25 | 0.062-32 [2] |
| CFM | 8 | >32 | 0.125 | 2 | 0.062-1.0 [0.125] |
| RFB | 0.25 | 2 | ≦0.031 | ≦0.0039 | 0.0078-0.125 [0.062] |
| RFZ | 0.062 | >32 | ≦0.031 | ≦0.0039 | ≦0.0039-0.0078 |
| | | | | | [≦0.0039] |
| AMK | 0.5 | 4 | 2 | 2 | 1->32 [16] |
| CIP | 0.5 | >32 | 0.25 | 2 | 0.125-32 [2] |
| CS | 32 | >32 | 8 | 16 | ND |
| MOX | 0.125 | >32 | 0.031 | 0.125 | ND |

Table 6 below shows the MIC of the capuramycin analogues of Table 3 against ten strains of MAC.

TABLE 6
Minimal inhibitory concentrations of CM analogues against MAC strains

| | MIC (μg/ml) | | | | | | | | | | | | | |
|---|---|---|---|---|---|---|---|---|---|---|---|---|---|---|
| MAC | | | | RKS | RKS | RKS | RKS | RKS | RKS | RKS | RS | | | |
| Strain | SQ997 | SQ922 | SQ641 | 2186 | 2033 | 2137 | 2243 | 2235 | 2244 | 2241 | 120029 | CLA | EMB | AMK |
| JSH1 | 32 | 16 | 2 | 4 | 2 | 1 | 1 | 2 | 0.50 | 2 | >32 | 2 | 16 | 8 |
| A5 | 16 | 2 | 0.50 | 1 | 2 | 1 | 0.5 | 1 | 0.25 | 1 | 32 | 0.125 | 1 | 4 |
| 100 | 16 | 4 | 2 | 2 | 2 | 2 | 1 | 2 | 0.50 | 2 | 32 | 0.25 | 4 | 8 |
| 101 | 8 | 1 | 0.12 | 2 | 2 | 1 | 1 | 1 | 0.25 | 1 | >32 | 0.125 | 1 | 2 |
| 104 | 16 | 4 | 2 | 1 | 1 | 1 | 1 | 1 | 0.25 | 0.5 | 32 | 0.5 | 2 | 16 |
| 109 | 8 | 0.5 | 0.12 | 1 | 1 | 0.5 | 0.5 | 1 | 0.25 | 0.5 | 32 | 0.062 | 0.5 | 1 |
| NIH C1-1 | 8 | >32 | 0.50 | 8 | 4 | 8 | 4 | 4 | 4 | 8 | >32 | 1 | 4 | 8 |
| NIH C1-2 | 4 | 1 | 0.25 | 2 | 2 | 2 | 1 | 2 | 0.50 | 1 | >32 | 0.25 | 2 | 32 |
| NIH C1-3 | 16 | 8 | 2 | 8 | 8 | 16 | 8 | 8 | 8 | 16 | >32 | 32 | 32 | 8 |
| NIH C1-4 | 8 | 2 | 0.50 | 2 | 2 | 2 | 1 | 2 | 1 | 2 | >32 | 1 | 2 | 4 |

Except for RS120029, all of the capuramycin analogues showed good activity across mycobacterial species (Table 5). Among the infective agents tested, MAC were more susceptible to the capuramycin analogues and MKN-12478 was the least susceptible. SQ641 was the most active compound against all the NTM tested, with a MIC that ranged from 0.125 to 2.0 μg/ml and a MIC₉₀ of 2 μg/ml (Table 5).

When the activities of the capuramycin analogues shown in Table 3 were compared against ten MAC isolates, SQ641 was the most active compound, with a MIC range of 0.125-2 μg/ml, and RKS2244 was the second most active compound, with a MIC range of 0.25-8 μg/ml (Table 6).

Minimum Bactericidal Activity (MBC). MBC of drugs against Mycobacteria was determined in 7H9 broth as described previously (Luna-Herrera et al., Antimicrob. Agents and Chemother. 1995, 39(2), 440-444). Drugs were diluted in 10 ml volumes to give 8× to 0.5× MIC. The drug-containing tubes and the drug-free control growth tube were inoculated with 0.1 ml of MTB suspension adjusted to 0.1 λ₆₀₀. An aliquot (0.5 ml) from the drug free control tube was diluted serially and plated on replicate 7H11 agar plates to determine initial CFU. The tubes were incubated at 37° C. in a shaker incubator for 2 wk. At the end of incubation, cultures showing no visible growth were diluted serially below the drug MIC to prevent drug carryover, and 0.1 ml aliquots were plated on replicate 7H1 1 agar plates. CFU were read after 3 wk incubation at 37° C. The lowest concentration of drug to kill 99.9% of initial MTB inoculum was the MBC (Heifets, Antituberculosis Drugs: Antimicrobial Activity in vitro. Drug Susceptibility in the Chemotherapy of Microbacterial Infections, 99, CRC Press, Boca Raton, Fla.). The MBC of CM analogues was determined against MSMG and MTB (H37Rv). The capuramycin analogues SQ641, SQ922 and SQ997 were bactericidal at their MIC and had a MBC/MIC ratio of 1.

Rate of killing. The luciferase gene-containing strain of H37Rv (MTB-pSMT1) after 1 wk growth was centrifuged and the pellet was resuspended in 7H9 broth. Turbidity was adjusted to 0.05 λ₆₀₀. The suspension was distributed in 10ml volumes in 50 ml tubes. CM analogues and other anti-TB drugs were dispensed to MTB suspensions to give 1×, 2×, 4× and 8× the MIC. A drug-free control growth tube was included in each experiment. The viable number of MTB at time 0 hr and at different times thereafter was determined by counting the relative light units (RLU) in a luminometer (Snewin et al. Infect. Immun. 1999, 67(9), 4586-4593). In time-kill studies (rate of killing by a drug), capuramycin analogues SQ641, SQ922 and SQ997analogues were tested at 1×, 2× and 4× MIC. The RLU, a function of the viable number of organisms, fell sharply in relation to time following exposure to capuramycin analogues. At 4× MIC, SQ641, SQ922 and SQ997 killed >50% of the organisms within 12 hr and >90% of the bacilli within 48 hr. By day 7, all the organisms were killed. SQ641 was more bactericidal than any other anti-TB drug. Unlike other anti-TB drugs, MTB killing by capuramycin analogues was evident as early as 4 hr.

Induction of morphological changes and bacterial disintegration. Capuramycin analogues SQ641, SQ922 and SQ997 caused a marked fall in the turbidity of MTB cultures within 2 days of exposure. Brief exposure to capuramycin analogues SQ641, SQ922 and SQ997 caused changes in morphology of MTB, but did not affect acid-fastness. Tubercle bacilli exposed to CM analogues were typically club-shaped, with deeply stained swollen ends (see Reddy et al. Antimicrob. Agents Chemother., 2008, 52(2), 719-721).

PAE of SQ641. PAE of SQ641 was determined as described (Chan et al. Antimicrob. Agents Chemother., 2004, 48(1), 340-343). At the end of incubation, suspensions were diluted 1/25, and 0.1 ml was injected into each of triplicate BACTEC vials: drug dilution was 1000-fold in the BACTEC vials. Drug carryover controls (to ascertain absence of any carryover drug) consisting of the same MTB suspension as in the test and similar drug dilutions were mixed immediately, diluted 1/25, and 0.1 ml was injected into BACTEC vials. BACTEC vials were incubated at 37° C. and read in a BACTEC TB-460 reader every 24 hr until the cumulative GI in the control reached 999. PAE was calculated from the formula PAE=T−C, where T is the time required for the drug-exposed organisms to reach a cumulative GI of 100 and C is the time required for the controls to reach a cumulative GI of 100.

PAE determines the time required for the organisms to recover following exposure to an antimicrobial agent for a short duration. PAE activities of both SQ641 and INH, each at 4 μg/ml, were investigated with MTB-H37Rv. Following 2 hr of exposure to drugs, the PAE of INH was 17 hr as compared to 55 hr for SQ641. The PAE of SQ641 was over 3 times that of INH.

Synergistic activity with other anti-TB drugs against MSMG, MTB, MAB, and MAC. Synergy with other anti-mycobacterial drugs was determined in two drug combinations by checkerboard titration using a microdilution method. Fractional inhibitory concentration (FIC) is the MIC in combination divided by the MIC of individual drug. An FIC index (ΣFIC) of ≦0.5 indicates synergistic activity, ≧4.0 antagonistic activity and in between, additive effect. Synergistic activity was determined against MSMG, MTB, MAB and MAC. All the three CM analogues were tested in combination with INH, RIF, EMB and SM against MSMG. Similarly, all the three CM analogues were tested in combination with INH, EMB, SM and SQ109 against MTB. Only SQ641 was tested in combination with EMB, clarithromycin (CLA) and amikacin (AMK) against MAC.

SQ641 displayed synergy in combination with INH and EMB in MSMG, with EMB, SQ109 and INH in MTB, and with EMB in MAC. The capuramycin analogue SQ641 was synergistic with EMB against all mycobacterial strains tested, with maximum effects against MTB (ΣFIC=0.156). Among the capuramycin analogues tested, only SQ641 showed synergy with other antimycobacterial drugs against NTM. Table 7 below shows the synergy between SQ641 and other antimycobacterial agents against MAC, MAB and MSMG. MAB was highly resistant to EMB. Typical checkerboard titrations between SQ641 and EMB against three MAC strains are shown in Tables 8-10 below. It is interesting to note that SQ641 showed synergistic activity with EMB even in an EMB-resistant MAC strain (JSH1).MAB was highly resistant to EMB.

TABLE 7
Synergy between SQ641 and other antimycobacterial agents in
against non-tuberculosis mycobacteria (NTM)

| | Drug | MIC (μg/ml) | | |
|---|---|---|---|---|
| Organism | combination | Alone | Combination | ΣFIC |
| MAC A5 | SQ641 | 0.25 | 0.125 | 0.75 |
| | CLA | 0.125 | 0.031 | |
| | SQ641 | 0.25 | 0.125 | 0.75 |
| | AMK | 2.0 | 0.5 | |
| | SQ641 | 0.25 | 0.062 | 0.31 |
| | EMB | 1.0 | 0.062 | |
| MAC JSH1 | SQ641 | 1.0 | 1 | 2.0 |
| | CLA | 1 | 1 | |
| | SQ641 | 1.0 | 0.125 | 0.625 |
| | AMK | 16 | 8.0 | |
| | SQ641 | 1.0 | 0.25 | 0.5 |
| | EMB | 32.0 | 8.0 | |
| MAC NIH | SQ641 | 2.0 | 2.0 | 2.0 |
| C1-1 | CLA | 2.0 | 2.0 | |
| | SQ641 | 2.0 | 0.5 | 1.0 |
| | AMK | 8.0 | 4.0 | |
| | SQ641 | 2.0 | 0.06 | 0.281 |
| | EMB | 8.0 | 2.0 | |
| M. abscessus | SQ641 | 0.5 | 0.062 | 0.375 |
| | SM | 8.0 | 2.0 | |
| | SQ641 | 0.5 | 0.062 | 0.25 |
| | RFB | 2.0 | 0.25 | |
| M. smegmatis | SQ641 | 2.0 | 0.25 | 0.25 |
| | INH | 8.0 | 1.0 | |
| | SQ641 | 2.0 | 1.0 | 0.75 |
| | RIF | 8.0 | 2.0 | |
| | SQ641 | 2.0 | 1.0 | 1.0 |
| | SM | 0.5 | 0.25 | |
| | SQ641 | 2.0 | 0.25 | 0.375 |
| | EMB | 0.5 | 0.12 | |

Tables 8-10 below shows a typical checkerboard titrations between SQ641 and EMB against three strains of MAC.

TABLE 8
Synergistic Activity Between SQ641 and EMB Against MAC A5
Strain
MAC A5; ΣFIC = 0.281
SQ641

| | μg/ml | 0 | 0.062 | 0.125 | 0.25 | 0.5 | 1 | 2 | 4 |
|---|---|---|---|---|---|---|---|---|---|
| EMB | 0 | + | + | + | + | + | + | − | − |
| | 0.25 | + | + | + | + | + | + | − | − |
| | 0.5 | + | + | + | + | + | + | − | − |
| | 1 | + | + | + | + | + | + | − | − |
| | 2 | + | + | + | + | − | − | − | − |
| | 4 | + | − | − | − | − | − | − | − |
| | 8 | − | − | − | − | − | − | − | − |
| | 16 | − | − | − | − | − | − | − | − |

TABLE 9
Synergistic Activity Between SQ641 and EMB Against MAC JSH1
Strain
MAC JSH1; ΣFIC = 0.5
SQ641

| | μg/ml | 0 | 0.031 | 0.062 | 0.125 | 0.25 | 0.5 | 1 | 2 |
|---|---|---|---|---|---|---|---|---|---|
| EMB | 0 | + | + | + | + | + | + | − | − |
| | 0.5 | + | + | + | + | + | + | − | − |
| | 1 | + | + | + | + | + | + | − | − |
| | 2 | + | + | + | + | + | + | − | − |
| | 4 | + | + | + | + | + | + | − | − |
| | 8 | + | + | + | + | − | − | − | − |
| | 16 | + | + | + | + | − | − | − | − |
| | 32 | − | − | − | − | − | − | − | − |

TABLE 10
Synergistic Activity Between SQ641 and EMB Against MAC NIH Cl-1
Strain
MAC NIH Cl-1; ΣFIC = 0.281
SQ641

| | μg/ml | 0 | 0.062 | 0.125 | 0.25 | 0.5 | 1 | 2 | 4 |
|---|---|---|---|---|---|---|---|---|---|
| EMB | 0 | + | + | + | + | + | + | − | − |
| | 0.5 | + | + | + | + | + | + | − | − |
| | 1 | + | + | + | + | + | + | − | − |
| | 2 | + | + | + | + | − | − | − | − |
| | 4 | + | − | − | − | − | − | − | − |
| | 8 | − | − | − | − | − | − | − | − |
| | 16 | − | − | − | − | − | − | − | − |
| | 32 | − | − | − | − | − | − | − | − |

Activity in acidic pH. The pH in macrophage phagolysomes is ˜5.5. To be effective against intracellular organisms, anti-TB drugs should concentrate inside macrophages and be active in an acidic environment. The activity of SQ641 against MTB was investigated by microdilution method in 7H9 broth at different hydrogen ion concentrations. It was found that there was no loss of SQ641 activity from pH 7.0 to 5.0.

Activity against slowly replicating MTB. The microenvironment within the tubercle lesions is purportedly hypoxic and organisms persist in such an environment without replication or by slow replication (semi-dormant). The MTB-pSMT1 luciferase reporter strain was used to determine the susceptibility of hypoxic MTB by microdilution in 7H9 broth. Plates were incubated in anaerobic conditions using Becton Dickinson GasPak EZ anaerobic system. The viable number of MTB was determined by estimating the relative light units (RLU) of the culture at the beginning and at day 7 following exposure to drugs. SQ641 maintained its activity in hypoxic conditions (Table 11).

TABLE 11
Activity against slowly replicating MTB

MIC (μg/ml)

| Drug | Aerobic | Hypoxic |
|---|---|---|
| RIF | 0.031 | 0.0039 |
| INH | 0.062 | 0.125 |
| SM | 0.25 | 1.0 |
| EMB | 2.0 | 32 |
| SQ641 | 0.5 | 1.0 |

Growth rate was 24x in aerobic, 5x in hypoxic medium.

Development of drug resistant mutants. The frequency of development of spontaneous drug resistant mutants against SQ641 and RIF was determined as described previously (David App. Microbiol., 1970, 20(5), 810-814). In the first experiment, 10 μg/ml concentration of SQ641 and 1 μg/ml of RIF were used to select resistant mutants. Concentrations of drugs were equivalent to 10× MIC SQ641 and 16× MIC RIF. From 39 cultures inoculated on RIF plates there were 7178 colonies with a frequency of mutation of 3.96×10⁻¹¹. From 37 cultures inoculated on SQ641 plates no colonies grew, indicating: zero frequency of mutation. In a second experiment, 49 cultures of MTB were plated on 7H11 agar plates containing 8 μg/ml SQ641 and 45 cultures on plates containing 1 μg/ml RIF. Again, no colonies were detected in SQ641 plates, indicating zero frequency of mutation. With RIF, 6646 colonies grew out, a frequency of mutation of 2.4×10⁻¹². Even at lower concentrations (8× MIC) of SQ641 no mutants were observed, indicating its high efficacy in preventing development of drug resistance.

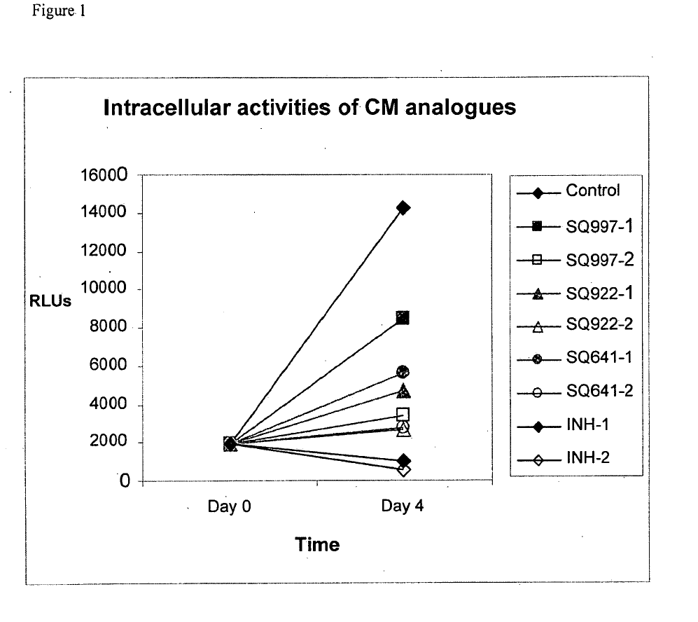

Intracellular activity of DMSO-solubilized SQ641. To determine the intracellular activities of drugs, MTB infected macrophages were incubated with capuramycin analogues SQ641, SQ922 and SQ997 at 1× and 2× MIC for 4 days, the cells were replaced with fresh medium and incubated for 3 more days. Infected macrophages were lysed at day 0 and 7 and the relative light units (RLU) were determined. In some experiments the cells were lysed and RLU determined on day 4. Despite high in vitro activity against MTB in culture, CM analogues were less efficacious in killing MTB inside macrophages. SQ922 and SQ641 showed intracellular activities at 1× and 2× MIC (FIG. 1). Neither analogue showed toxicity to macrophages even at 64 and 32 μg/ml concentrations, respectively. A dose response for SQ922 and SQ641 against MTB inside macrophages was determined (Table. 12). However, even at highest concentrations, activities were far below that of 2× MIC (0.25 μg/ml) INH.

TABLE 12
Intracellular activities of CM analogues: Dose
response.

| | |
|---|---|
| Conc. | Mean RLU at |

| Group | μg/ml | X MIC | Day 0 | Day 7 |
|---|---|---|---|---|
| SQ641-32 | 32 | 32 | 443 | 190 |
| SQ641-16 | 16 | 16 | 443 | 253 |
| SQ641-8 | 8 | 8 | 443 | 441 |
| SQ641-4 | 4 | 4 | 443 | 1116 |
| SQ641-2 | 2 | 2 | 443 | 1162 |
| SQ641-1 | 1 | 1 | 443 | 1208 |
| SQ922-64 | 64 | 32 | 443 | 134 |
| SQ922-32 | 32 | 16 | 443 | 311 |
| SQ922-16 | 16 | 8 | 443 | 496 |
| SQ922-8 | 8 | 4 | 443 | 1111 |
| SQ922-4 | 4 | 2 | 443 | 1080 |
| SQ922-2 | 2 | 1 | 443 | 1164 |
| INH-0.25 | 0.25 | 2 | 443 | 11 |
| Control | 0 | — | 443 | 4251 |

P-gp efflux pump blockers enhanced intracellular activity of SQ641. To determine whether the reduced intracellular activity of SQ641 was due to extrusion of the drug by efflux pumps, the activity of SQ641 was determined in the presence of the MDR1 efflux pump blockers verapamil (VE) and cyclosporine A (CsA), and multidrug resistance associated protein 1 (MRP1) blockers probenecid (PB) and gemfibrozil (GF). Both VE and CsA at 10 μM significantly enhanced intracellular activity of SQ641 against MTB in J774A.1 cells (Tables 13 and 14), whereas PB and GF had only moderate effects (data not shown). Neither VE nor CsA alone have antibacterial activity against MTB, nor do they enhance the activity of SQ641 in vitro. The effect of the P-gp blockers on the activity of SQ641 is shown in Tables 13 and 14. It was concluded that increased intracellular activity of SQ641 was due to the increased accumulation of the drug within macrophages.

TABLE 13
Enhancement of activity by verapamil (VE): Exposure
for 4 days to drugs.

| | |
|---|---|
| Conc. | Mean RLU at |

| Group | μg/ml | Day 0 | Day 7 |
|---|---|---|---|
| SQ641-32 | 32 | 425 | 141 |
| SQ641-32 + VE10 | 32 + 5 | 425 | 12 |
| SQ641-16 | 16 | 425 | 654 |
| SQ641-16 + VE10 | 16 + 5 | 425 | 18 |
| SQ641-8 | 8 | 425 | 1392 |
| SQ641-8 + VE10 | 8 + 5 | 425 | 44 |
| SQ641-4 | 4 | 425 | 2329 |
| SQ641-4 + VE10 | 4 + 5 | 425 | 368 |
| SQ641-2 | 2 | 425 | 2362 |
| SQ641-2 + VE10 | 2 + 5 | 425 | 509 |
| SQ641-1 | 1 | 425 | 3866 |
| SQ641-1 + VE10 | 1 + 5 | 425 | 686 |
| INH-0.25 | 0.25 | 425 | 127 |
| VE 10 | 5 | 425 | 5313 |
| Control | 0 | 425 | 6283 |

TABLE 14
Enhancement of activity by cyclosporine A: Exposure
to drugs for 4 days

| | |
|---|---|
| Conc. | Mean RLU at |

| Group | μg/ml | Day 0 | Day 7 |
|---|---|---|---|
| SQ641-32 | 32 | 811 | 113 |
| SQ641-32 + CsA10 | 32 + 12 | 811 | 11 |
| SQ641-16 | 16 | 811 | 346 |
| SQ641-16 + CsA10 | 16 + 12 | 811 | 14 |
| SQ641-8 | 8 | 811 | 850 |
| SQ641-8 + CsA10 | 8 + 12 | 811 | 15 |
| SQ641-4 | 4 | 811 | 1476 |
| SQ641-4 + CsA10 | 4 + 12 | 811 | 28 |
| SQ641-2 | 2 | 811 | 1361 |
| SQ641-2 + CsA10 | 2 + 12 | 811 | 43 |
| SQ641-1 | 1 | 811 | 2233 |
| SQ641-1 + CsA10 | 1 + 12 | 811 | 342 |
| INH-0.25 | 0.25 | 811 | 20 |
| CsA10 | 12 | 811 | 2023 |
| Control | 0 | 811 | 3004 |

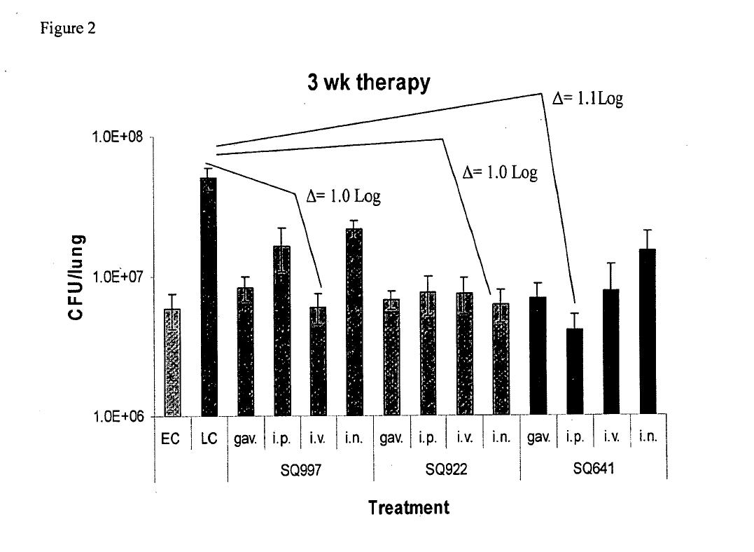

In vivo activity against MTB. In vivo efficacy of CM analogues was investigated in a mouse model of chronic TB disease (FIG. 2). Three wk after infection, mice were treated for 3 wk with the drugs by gavage (gav), intraperitoneal injection (ip), intravenous injection (iv), or intranasal (in) routes. Efficacy was analyzed by determining Colony-Forming Units (CFU) in lungs. The three compounds were able to prevent replication of MTB (compared with the early control [EC]: measurement of CFU in lungs at the beginning of treatment) by gav and iv for SQ997; most routes for SQ922, and all but intranasal for SQ641. Each achieved a decrease of 1-log₁₀ lung CFU compared to the late control [LC] CFU by at least one route, which varied for each drug.

### Example 2

### Amino Acid Derivatives of Capuramycin Analogues

As shown in Schemes 1 and 2, amino acid derivatives of capuramycin analogues at were prepared from the compounds SQ997 and SQ641. Starting from SQ-997, diol functionality was protected as the ketal using acetone dimethylacetal under acidic conditions, and the resulting protected compound was treated with dicycohexylcarbodiimide (DCC) and reacted with a Boc-protected amino acid in the presence of 4-dimethylaminopyridine (DMAP) as catalyst. The product was deprotected with 5% trifluoroacetic acid in dichloromethane to form the desired amino acid derivative.

The 2′- and 3′-hydroxy groups in SQ-641 are blocked and therefore, derivitization of the hydroxy groups on the dihydropyran ring was necessary to form the amino acid derivatives. Coupling conditions similar to those used for SQ-997 were used to form the SQ-641 amino acid derivatives. Briefly, to a solution of SQ641 in THF 11-(Boc-amino)undecanoic acid was added followed by an addition of dimethylaminopyridine (DMAP). The reaction mixture was cooled to 0° C. in an ice bath, and dicyclohexylcarbodiimide (DCC) was added. Then the ice bath was removed, and the reaction mixture was stirred at room temperature over night. The reaction mixture was worked up (standard aqueous work-up), and the product was purified by flash column chromatography. Mass spectrometry was used to confirm formation of the desired compound.

The SQ997 amino acid derivatives shown in Table 2 were tested for in vitro activity against MTB, cytotoxicity for uninfected J774 cells to determine IC₅₀, and for intracellular activity in the macrophage assay (Table 15). All amino acid derivatives showed MIC equal to or better than the parent compound SQ997. Cell viability remained similar to SQ997, or slightly better as cell morphologic changes were reduced.

TABLE 15
Mol. weights, chemical yields, MIC and cytotoxicity data for SQ997-
amino acid conjugates.

| | | | MIC, | |
|---|---|---|---|---|
| Compound name | FW | Yield, % | μg/ml¹ | IC₅₀, μg/ml² |
| SQ997 | 583.55 | | 8 | >32 |
| 997-isonipecotic acid | 694.69 | 38 | 8 | >32 |
| 997-aminooctanoic acid | 724.76 | 35 | 8 | >32 |
| 997-aminoundecanoic acid | 766.85 | 65 | 2 | 32 |
| 997-D-Val | 682.68 | 28 | 12.5 | >32 |
| 997-L-Val | 682.68 | 32 | 12.5 | >32 |
| 997-isonicotinic acid | 688.65 | 30 | 4 | >32 |

¹Determined against MTB H37Rv by broth microdilution.
²Determined using J774 macrophage cell line.

Table 16 below shows the intracellular activity of the SQ641 amino acid conjugates shown in Table 2. The amino acid conjugates were tested at concentrations of 4 mg/mL and 8 mg/mL compared with a control and isoniazid (INH).

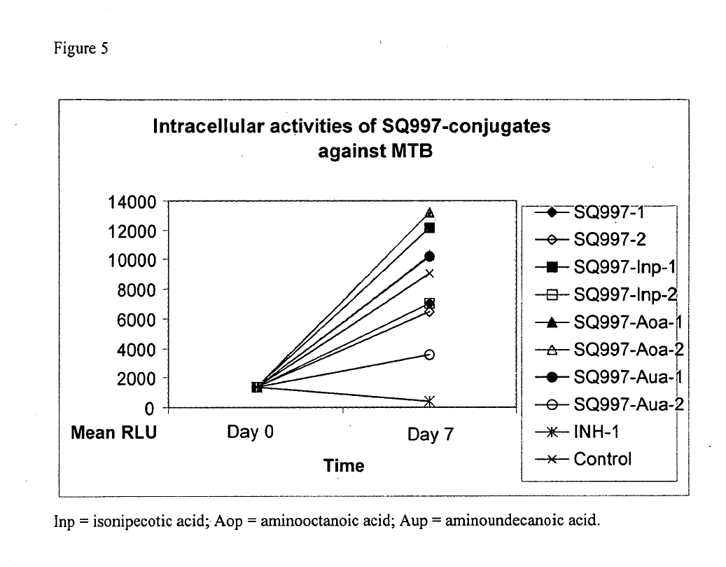

The intracellular activity against MTB by capuramycin amino acid derivatives was tested. Cells J774 were infected with MTB and treated for 7 days with the amino acid derivative. The mean Relative Light Unites (RLU), an estimation of bacterial viability, was measured as described in Snewin et al., Infect. Immun., 1999, 67(9), 4586-4593). FIG. 5 shows the intracellular activity of the amino acid derivatives against MTB.

TABLE 16
Intracellular activities of SQ641-
amino acid conjugates against MTB

| | |
|---|---|
| | Mean RLU |
| Conc. | on day |

| Drug | μg/ml | 0 | 7 |
|---|---|---|---|
| SQ641-4 | 4 | 775 | 4707 |
| SQ641-8 | 8 | 775 | 2755 |
| SQ641-aua-4 | 4 | 775 | 3163 |
| SQ641-aua-8 | 8 | 775 | 372 |
| SQ641-topdec-4 | 4 | 775 | 3368 |
| SQ641-topdec-8 | 8 | 775 | 2143 |
| SQ641-2topdec-4 | 4 | 775 | 4354 |
| SQ641-2topdec-8 | 8 | 775 | 2903 |
| INH-1 | 0.125 | 775 | 131 |
| Control | 0 | 775 | 31700 |

-aua = aminoundecanoic acid;
-topdec = 15-(amino)-4,7,10,13-tetraoxapentadecanoic acid.
-2topdec = two molecules of -topdec.

### Example 3

### Polyethylene Glycol Derivatives of Capuramycin Analogues

Compound SQ997 (shown in Scheme 1), was reacted with two PEG reagents comprising a terminal carboxylate functional group using DCC and DMAP as catalyst, as described for the preparation of amino acid derivatives in Example 2. Conjugates of SQ997 with PEG groups of 3 kDa and 5 kDa molecular weights were formed. The structure of the resulting conjugates was confirmed by NMR (600 MHz, CDCl₃), and the content of SQ997 in the conjugate was determined by HPLC. PEG has no chromophores and is not detectable by Diode Array Detector (DAD), so SQ997 and the PEG-SQ997 derivate could be quantitatively analyzed by HPLC using DAD with maximum absorption at 260 nm and additional absorption at 280 nm. A calibration curve was prepared using SQ997 in the concentration range 0.5-0.01 mg/ml. Then 4 weighted samples from the same batch of SQ997-PEG conjugates were analyzed, DAD signals were recorded and compared to the calibration curve. The average loading of SQ997 on PEG was estimated to be in the range 0.02-0.03 mg per 1 g of conjugate, which corresponds to 20-33% loading.

The SQ997 PEG conjugates were evaluated for intracellular activity against Mycobacterium tuberculosis compared to unconjugated SQ997 at 8 and 16 μg/mL. Table 17 below shows the activity of the PEG-conjugated compounds as compared with unconjugated SQ997 and INH.

TABLE 17
Intracellular activity of SQ997-
PEG conjugate against MTB.

Mean RLU

| Group | Day 0 | Day 7 |
|---|---|---|
| SQ997-8 | 255 | 1080 |
| SQ997-16 | 255 | 410 |
| SQ997-PEG-DMSO-8 | 255 | 396 |
| SQ997-PEG-DMSO-16 | 255 | 127 |
| SQ997-PEG-H20-8 | 255 | 451 |
| SQ997-PEG-H20-16 | 255 | 292 |
| INH-0.125 | 255 | 4 |
| Control | 255 | 1856 |

### Example 4

### Formulations in D-Tocopheryl Polyethylene Glycol 1000 Succinate

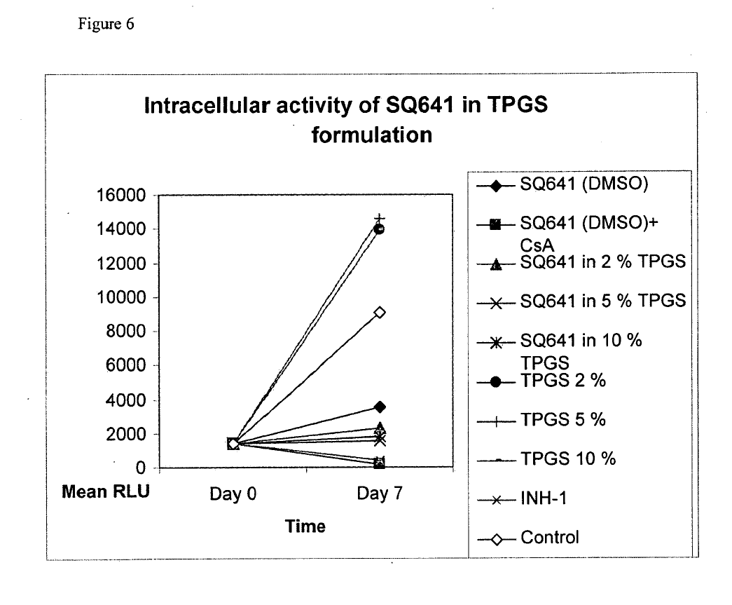

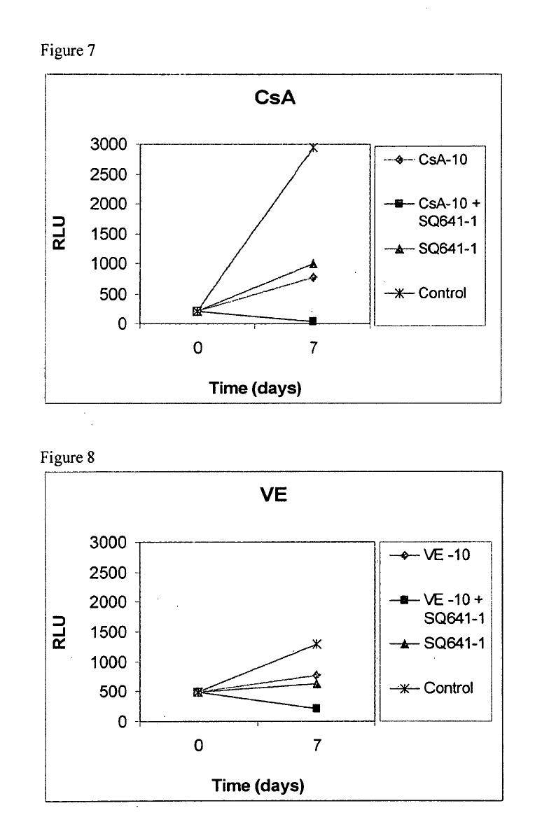

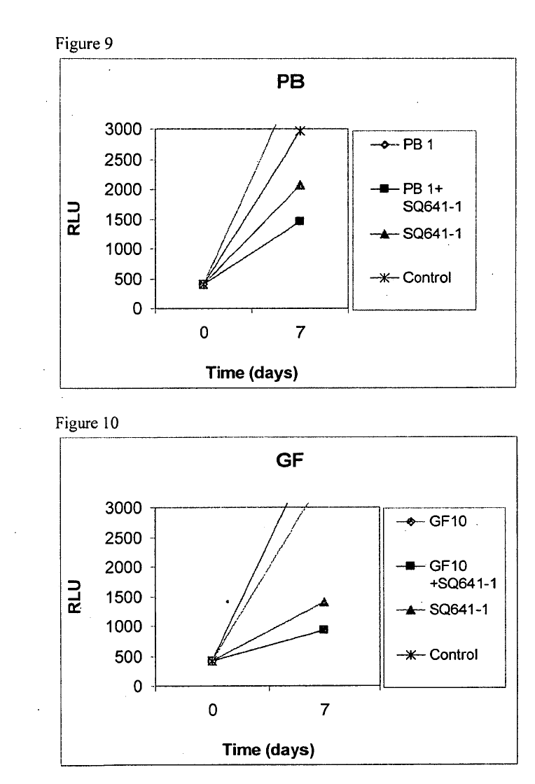

Formulations in D-Tocopheryl polyethylene glycol 1000 Succinate (TTPGS) is a water soluble vitamin E analogue used to improve solubility and absorption of many pharmaceutical agents (EASTMAN and Company 2005). SQ641 was found to be completely soluble in ≧2% TPGS. Moreover, as FIG. 6 demonstrates, SQ641 dissolved in TPGS showed improved intracellular activity as compared to drug dissolved in DMSO. TPGS is also a P-gp blocker. Whether TPGS-facilitated SQ641 activity is due to increased membrane permeability or blocking of P-gp mediated drug efflux is not clear. Interestingly, TPGS alone increased MTB growth in macrophages.

Intracellular activity of SQ641 in TPGS. The efficacy of SQ641 solubilized in TPGS was investigated, and it was found TPGS solutions of SQ641 exhibited improved intracellular activity compared to drug dissolved in DMSO (Table 18). The RLU in presence of SQ641 dissolved in 5 or 10% TPGS was about 50% lower than the drug dissolved in DMSO.

TABLE 18
Intracellular efficacy of SQ641 dissolved in TPGS

| | |
|---|---|
| | Mean RLU |
| Conc. | on day |

| Formulation | μg/ml | 0 | 7 |
|---|---|---|---|
| SQ641 | 2 | 1397 | 3535 |
| SQ641 + TPGS | 2 + 4 | 1397 | 2290 |
| SQ641 + TPGS | 2 + 10 | 1397 | 1556 |
| SQ641 + TPGS | 2 + 20 | 1397 | 1845 |
| TPGS | 4 | 1397 | 13903 |
| TPGS | 10 | 1397 | 14595 |
| TPGS | 20 | 1397 | 13942 |
| INH-1 | 0.125 | 1397 | 434 |
| Control | 0 | 1397 | 9075 |

Activity in vivo of soluble SQ641 in TPGS. The in vivo activity of TPGS-solubilized SQ641 by oral and IP routes against MTB in a chronic infection mouse model was investigated. Three weeks following infection, mice were treated for 2 wk with SQ641-TPGS at 2.5 mg/kg, IP; SQ641-TPGS 25 mg, PO; TPGS 250 mg/kg, IP; or INH 25 mg/kg, PO. The lung CFU data (Table 19) suggested that TPGS-SQ641 was not absorbed orally, but soluble SQ641had excellent activity delivered systemically by IP injection, and was equivalent to or better than INH.

TABLE 19
In vivo activity of TPGS-solubilized SQ641 in MTB chronic infection
model: Lung CFU following 2 weeks treatment.

| | | Dose | | |
|---|---|---|---|---|
| Group | Route | (mg/kg) | CFU ± SD | Log CFU |
| Control | | — | 3.8 ± 1.9 × 10⁸ | 8.58 |
| TPGS 2.5% | IP | 250 | 3.0 ± 1.3 × 10⁸ | 8.48 |
| SQ641 in TPGS | IP | 2.5 | 7.7 ± 2.0 × 10⁶ | 6.89* |
| SQ641 in TPGS | PO | 25 | 1.5 ± 0.7 × 10⁸ | 8.18 |
| INH | PO | 25 | 1.9 ± 0.8 × 10⁷ | 7.28 |

*P = 0.49

The data suggests that SQ641 in TPGS given parenterally was systemically available and potent.

SQ641-TPGS micelle formulation. SQ641 was poorly soluble in water, but very soluble in TPGS. While titrating to determine the lowest amount of TPGS required to solubilized SQ641, it was discovered that TPGS at 1:1 (wt:wt) concentration with sonication forms stable micelles without need for an emulsifier. Particle size and drug loading was controlled by varying the intensity of sonication, pH, molarity, temperature, and volume of the suspension. A 40% sucrose suspension and bath sonicator produced small rod shaped particles (FIG. 3). Smaller particles are shorter in length with the same diameter, which may be dictated by the length of two opposing molecules of TPGS).

Preparation and characterization of SQ641-TPGS micelles. Ten mg SQ641 was suspended in 10% TPGS prepared in 40% sucrose in a 4 ml polystyrene tube. The suspension was subjected to sonication in a water bath sonicator until formation of a white suspension. Sonication was extended for 1 more min. The suspension was centrifuged at 10000 rpm for 5 min. The supernatant was discarded and the pellet was washed twice with PBS. Bigger particles were separated by centrifugation at 1000 rpm for 5 minutes and supernatant fine particles were transferred to a fresh tube.

To determine the amount of drug entrapped in the micelles, 50 μl of the suspension was centrifuged at 10000 rpm for 5 min. The pellet was dissolved in 50 μl of DMSO and the drug was quantified by a microbiologic assay using M. smegmatis (a known concentration of free drug was used for comparison). To evaluate uptake of micelles by macrophages, J774A.1 macrophages were incubated with different concentrations of the small-particle and large-particle suspensions (FIG. 4). Cells were observed at 24 hr for phagocytosis.

To determine efficacy of SQ641-TPGS micelles against intracellular MTB, J774A.1 macrophages were cultured in 24-well tissue culture plates. Macrophages were infected with MTBp-SMT1 and incubated with different concentrations of SQ641-TPGS micelles or free drug. After 24 hr, wells were washed and tissue culture medium free of drug was added. Macrophages were lysed at day 7 and RLU determined. The change in mean RLU of infected macrophages treated with different preparations is compared in Table 20 (Expt. 1). In another experiment, infected macrophages were exposed to drugs for 4 days and RLU determined on day 7 post-infection (Table 20, Expt. 2). In both the experiments, SQ641-TPGS micelles showed 5-10 fold increase in activity compared to free drug and activity was comparable to INH. Significantly, this experiment demonstrated that SQ641 formed stable micellar emulsions with TPGS, and the drug was released inside the macrophages from endocytosed particles to kill MTB.

TABLE 20
Intracellular activity of SQ641-TPGS emulsion (E)

| | |
|---|---|
| Conc. | Mean RLU at |

| | | | |
|---|---|---|---|
| Group | μg/ml | Day 0 | Day 7 |

Expt 1. One day drug exposure

| | | | |
|---|---|---|---|
| SQ641-E20 | 20 | 104 | 4 |
| SQ641-20 | 20 | 104 | 47 |
| SQ641-E10 | 10 | 104 | 10 |
| SQ641-10 | 10 | 104 | 64 |
| SQ641-E5 | 5 | 104 | 23 |
| SQ641-5 | 5 | 104 | 95 |
| INH-4 | 0.5 | 104 | 8 |
| RIF-4 | 0.5 | 104 | 22 |
| CONTROL | 0 | 104 | 906 |

Expt 2. Four days drug exposure

| | | | |
|---|---|---|---|
| SQ641-E32 | 32 | 205 | 2 |
| SQ641-32 | 32 | 205 | 8 |
| SQ641-E16 | 16 | 205 | 2 |
| SQ641-16 | 16 | 205 | 17 |
| SQ641-E8 | 8 | 205 | 4 |
| SQ641-8 | 8 | 205 | 32 |
| SQ641-E4 | 4 | 205 | 11 |
| SQ641-4 | 4 | 205 | 62 |
| INH-2 | 0.25 | 205 | 1 |
| CONTROL | 0 | 205 | 491 |

SQ641-E = SQ641-TPGS micelle formulation

### Example 5

Safety profile. A series of IND-enabling cell- and mouse-based toxicology studies with the CM analogues in solution as solubility permitted, and in particles. No toxicity was observed with 4-day IV injections at doses of 1000 mg SQ997/kg/day and 500 mg SQ922/kg/day. Further toxicity studies of SQ641-TPGS solution in macrophages were conducted and observed no toxicity at 32 μg/ml of the drug. Toxicity studies with TPGS in mice found that 5 doses at ≦25 mg/mouse given IP daily were not toxic. Three doses of SQ641-TPGS micelles given at 1, 5, and 25 mg/kg IV; 10 and 50 mg/kg given IP; 50 and 100 mg/kg given PO were found to be non toxic. The spleens from these mice remained normal.

We claim:

1. An amino acid derivative of a capuramycin analogue of Formula Ia, or a pharmaceutically acceptable ether or ester thereof: 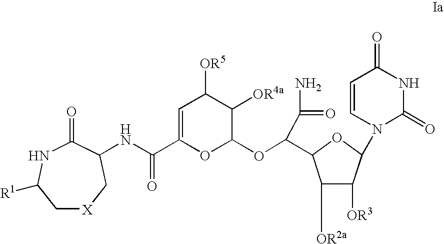
wherein R¹ is H or methyl;
X is CH₂ or S;
R^{4a} is H, a protecting group for a hydroxy group, or a residue of an amino acid;
R⁵, R^{2a} and R³ are independently H, methyl, alkanoyl, a protecting group for a hydroxy group, or a residue of an amino acid; and wherein at least one of R⁵, R^{2a}, R³ or R^{4a} is a residue of an amino acid.

2. The amino acid derivative of claim 1, wherein the derivative has the formula:

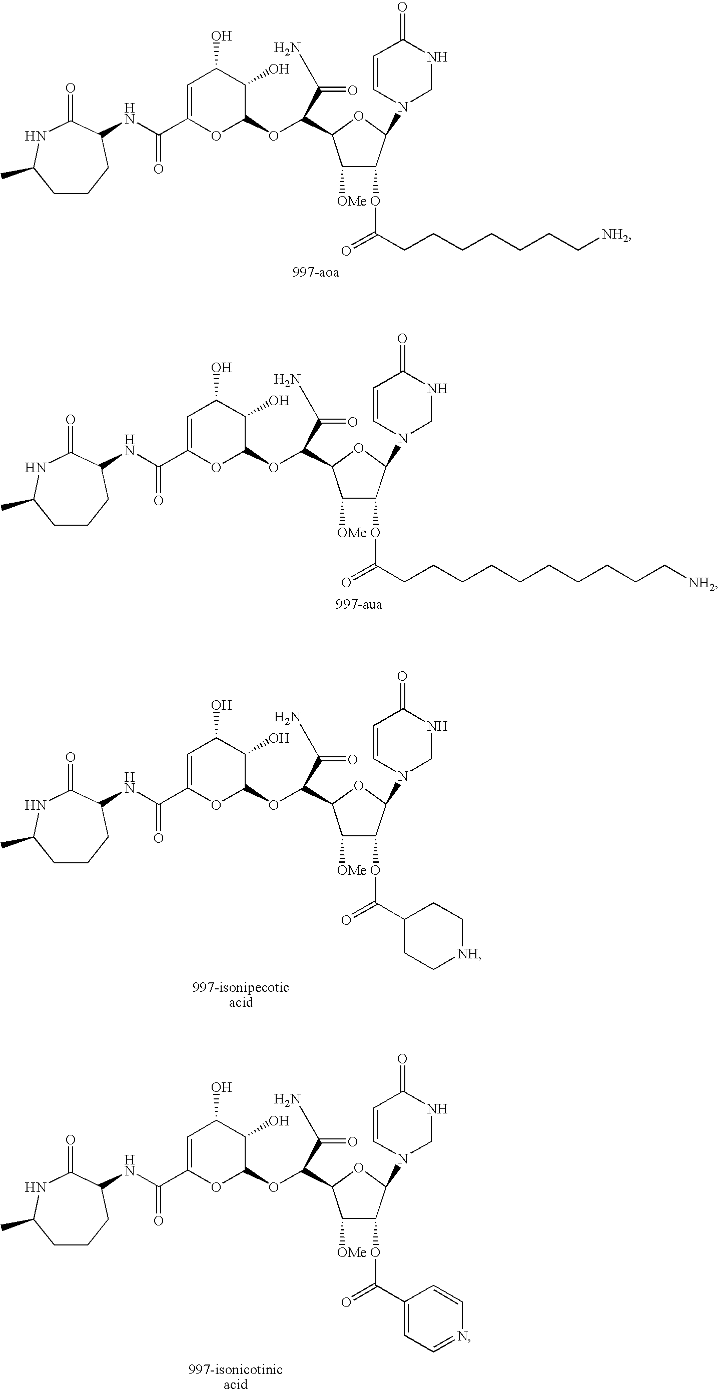

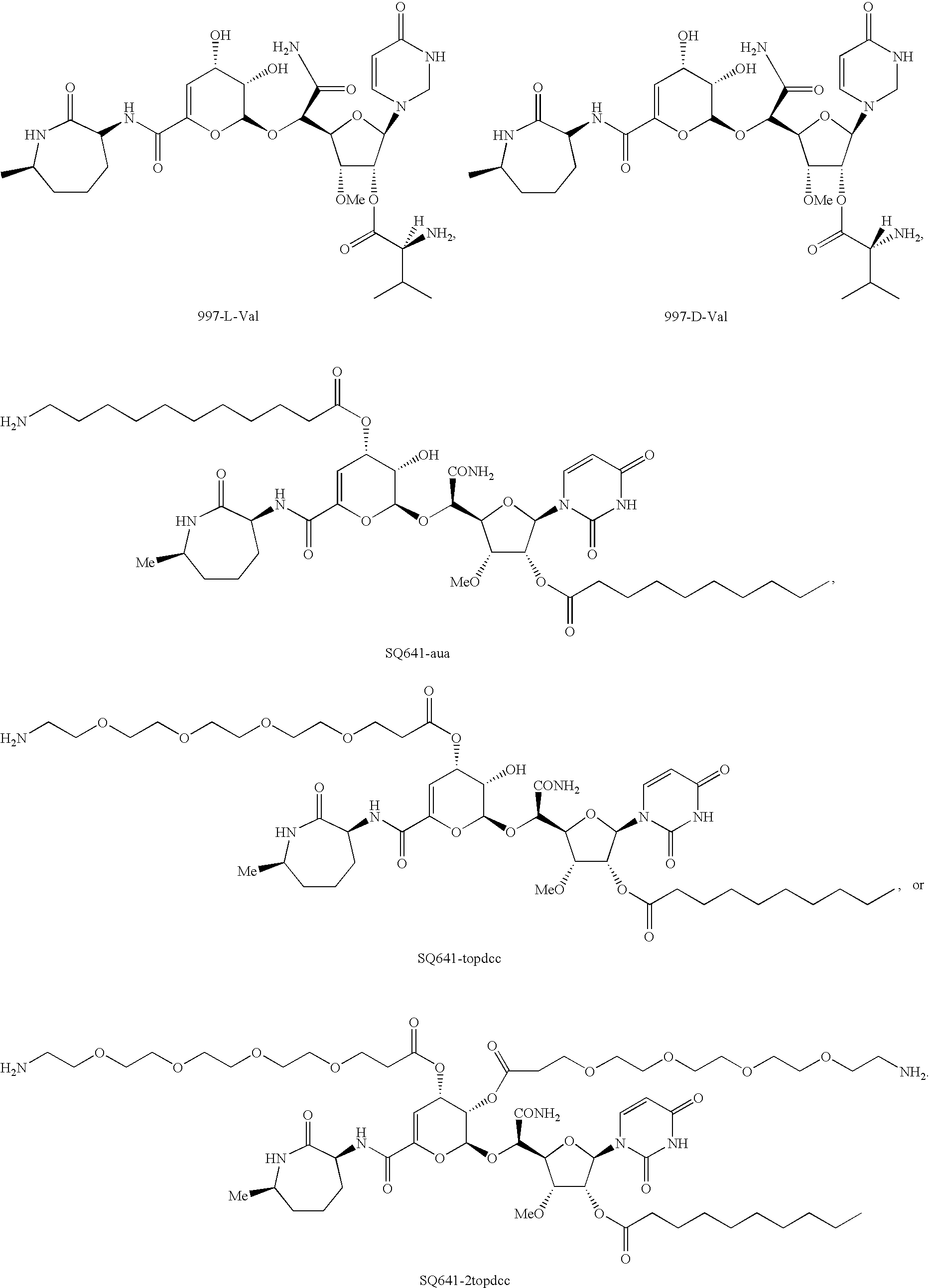

3. A PEGylated derivative of a capuramycin analogue of Formula Ib, or a pharmaceutically acceptable ether or ester thereof: 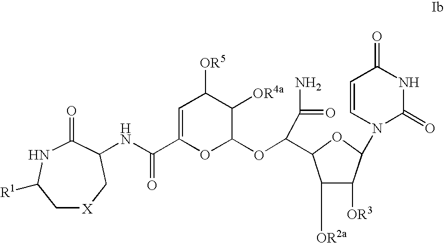
wherein R¹ is H or methyl;
X is CH₂ or S;
R^{4a} is H, a protecting group for a hydroxy group, or a group comprising a PEG residue; and
R⁵, R^{2a} and R³ are independently H, methyl, alkanoyl, a protecting group for a hydroxy group, or a group that comprises a PEG residue; where at least one of R⁵, R^{4a}, R^{2a} or R³ comprises a PEG residue.

4. The PEGylated derivative of claim 3, wherein the derivative has the structure 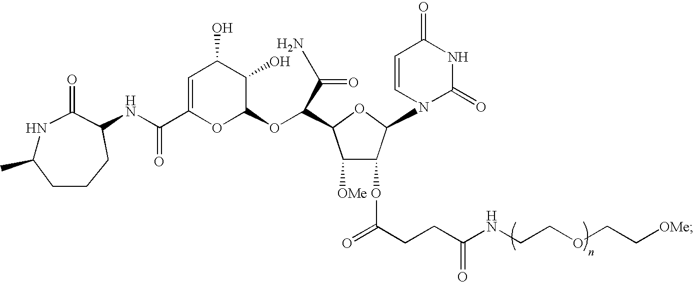
wherein n is 1-1000.

5. A formulation comprising: a capuramycin analogue of formula I-A or II, or pharmaceutically acceptable esters, ethers or N-alkylcarbamoyl derivatives thereof, 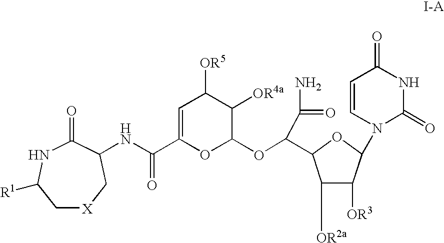
wherein R¹ is H or a methyl group,
R^{2a}, R³ or R⁵ are independently H, a protecting group for a hydroxy group, a methyl group, or an alkanoyl group;
R^{4a} is a H, or a protecting group for a hydroxy group; and
X is a methylene group or a sulfur atom; or a pharmaceutically acceptable salt or prodrug thereof; 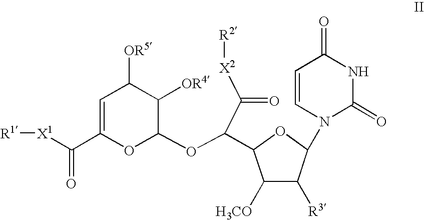
wherein X¹ represents an oxygen atom, a sulfur atom or —N(R⁶);
X² represents an oxygen atom, a sulfur atom or a —N(R⁷);
R^{1′} and R^{2′} are independently hydrogen; a methyl group; an optionally substituted aryl or heteroaryl group; an optionally substituted heterocyclic group; optionally substituted alkyl; an alkyl group which is substituted with one to three optionally substituted C₆-C₁₀ aryl groups, which may be the same or different; an alkyl group which is substituted with one to three optionally substituted heterocyclic groups, which may be the same or different; an optionally substituted alkenyl group; an optionally substituted alkynyl group; or a group of formula (a), 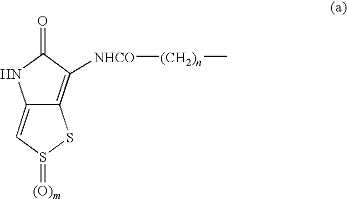
in which n is an integer from 1 to 20 and m is 0, 1 or 2; wherein each heterocyclic group has 1-4 nitrogen, sulfur or oxygen atoms;
R^{3′}, R^{4′} and R^{5′} are independently H, OH, alkanoyl, —O-alkanoyl, or a protecting group for a hydroxy group;
R⁶ is a hydrogen atom, a C₁-C₃ alkyl group, or R⁶, together with R¹ and the nitrogen atom to which they are attached, forms a 3- to 7-membered cyclic amine which may have a ring oxygen or sulfur atom; and
R⁷ is a hydrogen atom, a C₁-C₃ alkyl group, or R⁷, together with R^{2′} and the nitrogen atom to which they are attached, forms a 3- to 7-membered cyclic amine which may have a ring oxygen or sulfur atom;
wherein the aryl, heteroaryl, alkyl, alkenyl, or alkynyl groups are optionally substituted with acyl, alkyl, aryl, heterocyclic, halogen, hydroxyl, alkoxy, thiol, alkylthio, amino, alkyl or dialkylamino, nitro, cyano, carboxyl, carbamoyl, alkylenedioxy, aralkyloxy, CF₃ or —OCF₃;
and a hydrophobic organic compound that is derivatized with one or more PEG residues.

6. The formulation of claim 5, wherein the capuramycin analogue has the formula I-A.

7. The formulation of claim 5, wherein the capuramycin analogue has the formula II.

8. The formulation of claim 5, wherein the hydrophobic organic compound that is derivatized with one or more a PEG residues is D-alpha-tocopheryl poly(ethylene glycol) 1000 succinate.

9. A formulation comprising: a capuramycin analogue of formula I-A or II, or pharmaceutically acceptable esters, ethers or N-alkylcarbamoyl derivatives thereof, 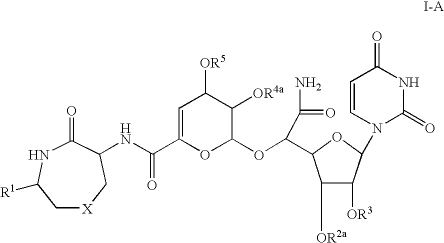
wherein R¹ is a hydrogen atom or a methyl group, R^{2a}, R³ or R⁵ are independently H, a protecting group for a hydroxy group, a methyl group, or an alkanoyl group; R^{4a} is a H, or a protecting group for a hydroxy group, and X is a methylene group or a sulfur atom, or a pharmaceutically acceptable salt or prodrug thereof; 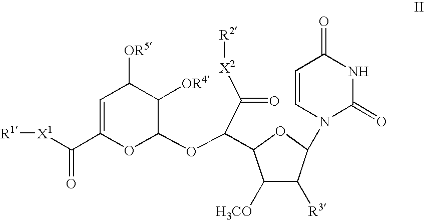
wherein X¹ represents an oxygen atom, a sulfur atom or —N(R⁶);
X² represents an oxygen atom, a sulfur atom or a —N(R⁷);
R^{1′} and R² are independently hydrogen; a methyl group; an optionally substituted aryl or heteroaryl group; an optionally substituted heterocyclic group; optionally substituted alkyl; an alkyl group which is substituted with one to three optionally substituted C₆-C₁₀ aryl groups, which may be the same or different; an alkyl group which is substituted with one to three optionally substituted heterocyclic groups, which may be the same or different; an optionally substituted alkenyl group; an optionally substituted alkynyl group; or a group of formula (a), 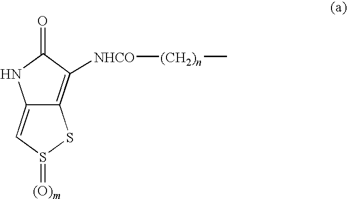
in which n is an integer from 1 to 20 and m is 0, 1 or 2; wherein each heterocyclic group has 1-4 nitrogen, sulfur or oxygen atoms;
R^{3′}, R^{4′} and R^{5′} are independently H, OH, alkanoyl, —O-alkanoyl, or a protecting group for a hydroxy group;
R⁶ is a hydrogen atom, a C₁-C₃ alkyl group, or R⁶, together with R^{1′} and the nitrogen atom to which they are attached, forms a 3- to 7-membered cyclic amine which may have a ring oxygen or sulfur atom; and
R⁷ is a hydrogen atom, a C₁-C₃ alkyl group, or R⁷, together with R^{2′} and the nitrogen atom to which they are attached, forms a 3- to 7-membered cyclic amine which may have a ring oxygen or sulfur atom;
wherein the aryl, heteroaryl, alkyl, alkenyl, or alkynyl groups are optionally substituted with acyl, alkyl, aryl, heterocyclic, halogen, hydroxyl, alkoxy, thiol, alkylthio, amino, alkyl or dialkylamino, nitro, cyano, carboxyl, carbamoyl, alkylenedioxy, aralkyloxy, CF₃ or —OCF₃;
and a pharmaceutically acceptable carrier; wherein the formulation is in the form of insoluble particulates.

10. The formulation of claim 9, wherein the capuramycin analogue has the formula I-A.

11. The formulation of claim 9, wherein the capuramycin analogue has the formula II.

12. The formulation of claim 9, wherein the insoluble particulates are micelles.

13. The formulation of claim 12, wherein the micelles comprise D-alpha-tocopheryl poly(ethylene glycol) 1000 succinate.

14. The formulation of claim 9, wherein the insoluble particulates are liposomes.

15. The formulation of claim 9, wherein the insoluble particulates comprise nanoparticle biocompatible polymers.

16. The formulation of claim 5, wherein the capuramycin analogue has the formula:

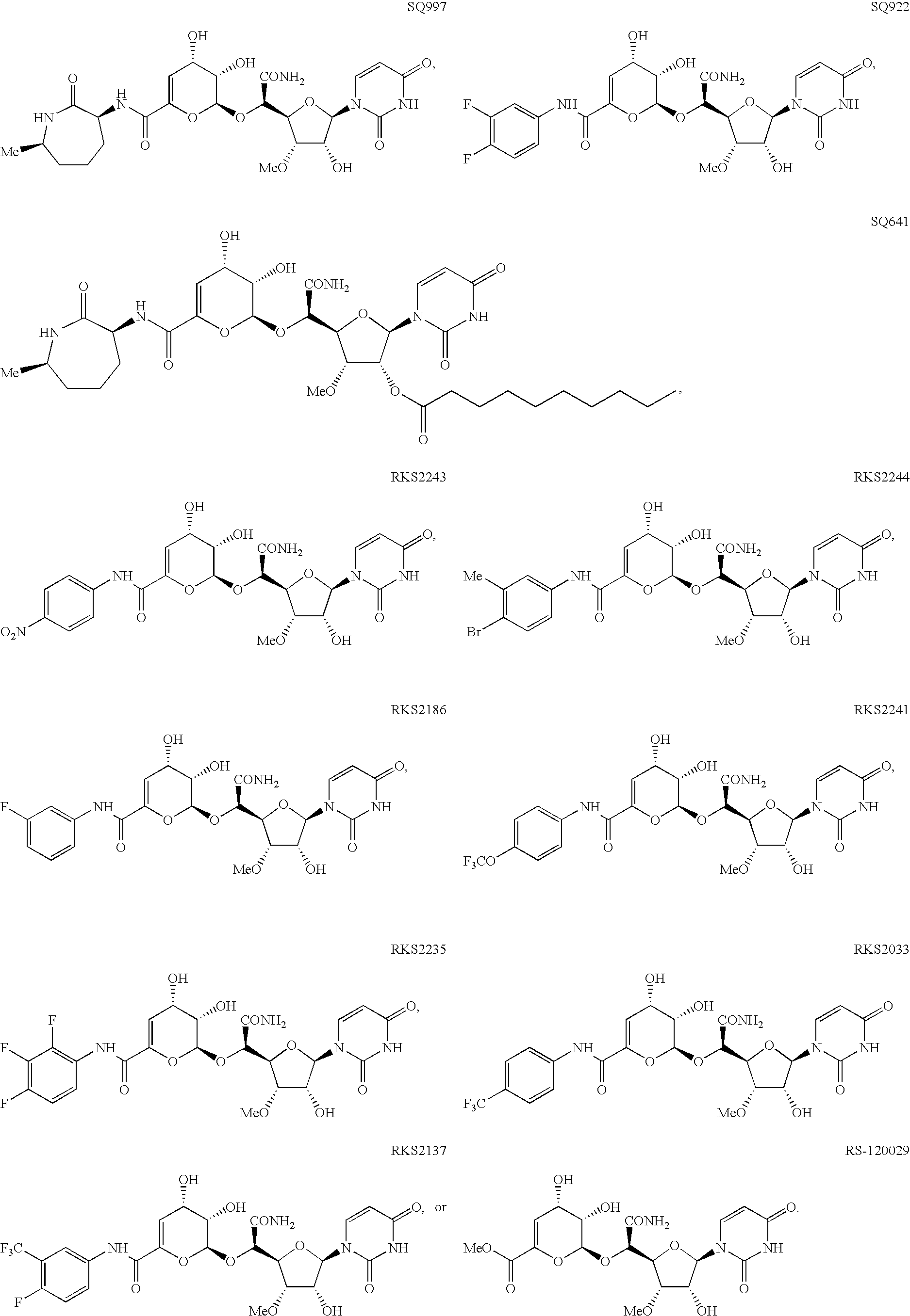

17. The formulation of claim 9, wherein the capuramycin analogue has the formula:

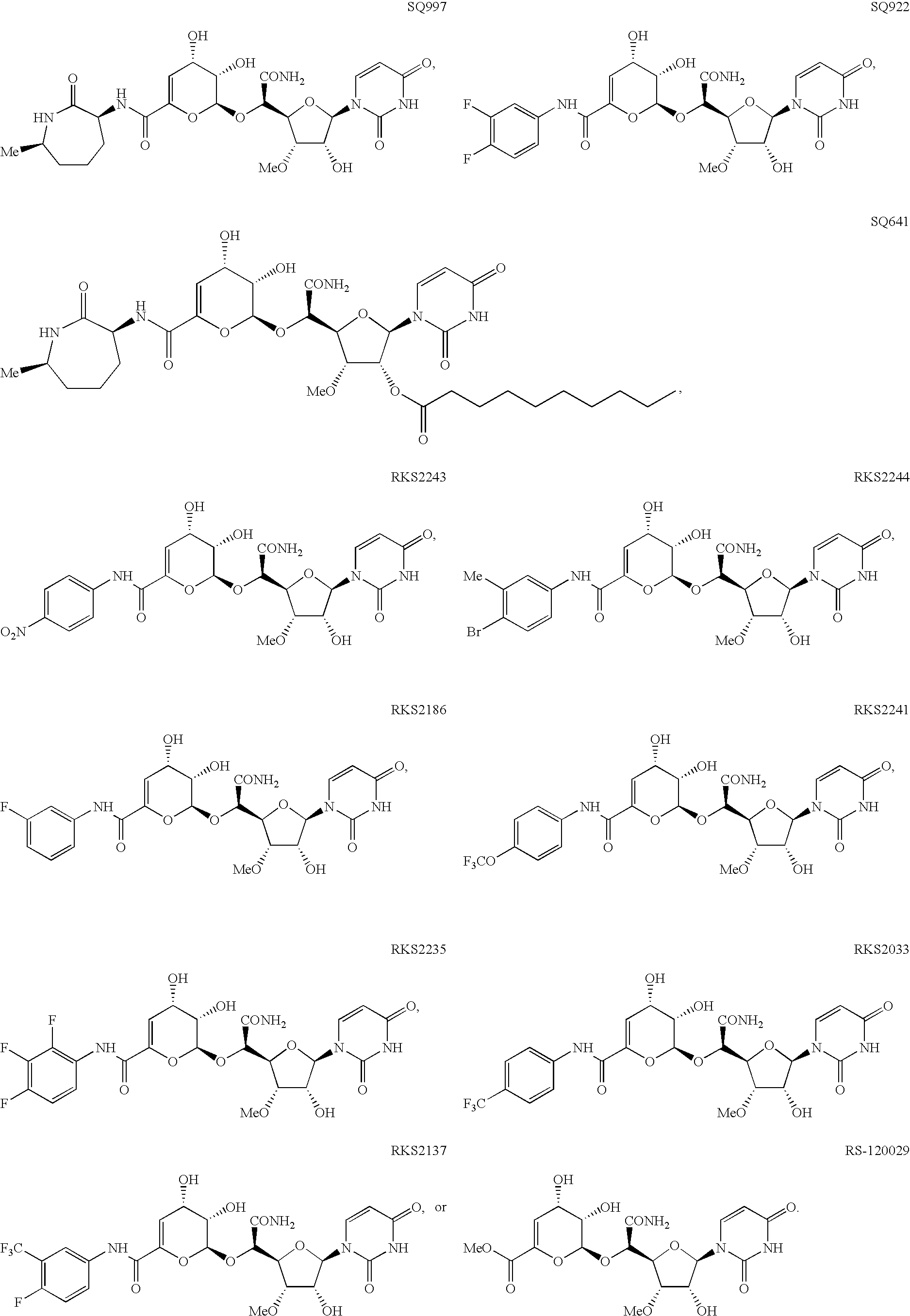

18. A method for the treatment of a mycobacterial disease comprising administering to a host with a mycobacterial disease the compound of claim 1.

19. A method for the treatment of a mycobacterial disease comprising administering to a host with a mycobacterial disease the compound of claim 3.

20. A method for the treatment of a mycobacterial disease comprising administering to a host with a mycobacterial disease the formulation of claim 5.

21. A method for the treatment of a mycobacterial disease comprising administering to a host with a mycobacterial disease the formulation of claim 9.

22. A method for the treatment of a mycobacterial disease comprising administering to a host with a mycobacterial disease a capuramycin analogue of formula I-A or II, or pharmaceutically acceptable esters, ethers or N-alkylcarbamoyl derivatives thereof: 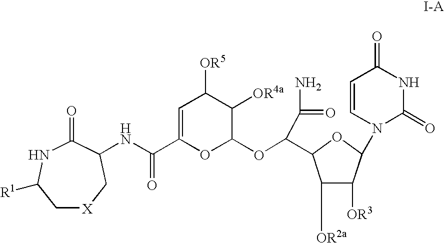
wherein R¹ is a hydrogen atom or a methyl group;
R^{2a}, R³ or R⁵ are independently H, a protecting group for a hydroxy group, a methyl group, or an alkanoyl group;
R^{4a} is H, or a protected hydroxy group; and
X is a methylene group or a sulfur atom; or a pharmaceutically acceptable salt or prodrug thereof; 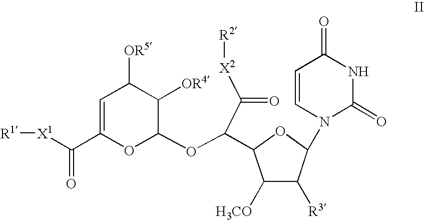
wherein X¹ represents an oxygen atom, a sulfur atom or —N(R⁶);
X² represents an oxygen atom, a sulfur atom or a —N(R⁷);
R^{1′} and R^{2′} are independently hydrogen; a methyl group; an optionally substituted aryl or heteroaryl group; an optionally substituted heterocyclic group; optionally substituted alkyl; an alkyl group which is substituted with one to three optionally substituted C₆-C₁₀ aryl groups; an alkyl group which is substituted with one to three optionally substituted heterocyclic groups, which may be the same or different; an optionally substituted alkenyl group; an optionally substituted alkynyl group; or a group of formula (a), 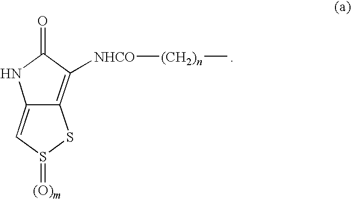
in which n is an integer from 1 to 20 and m is 0, 1 or 2; wherein each heterocyclic group has 1-4 nitrogen, sulfur or oxygen atoms; and
R^{3′}, R^{4′} and R^{5′} are independently H, OH, alkanoyl, —O-alkanoyl, or a protecting group for a hydroxy group;
wherein the aryl, heteroaryl, alkyl, alkenyl, or alkynyl groups are optionally substituted with acyl, alkyl, aryl, heterocyclic, halogen, hydroxyl, alkoxy, thiol, alkylthio, amino, alkyl or dialkylamino, nitro, cyano, carboxyl, carbamoyl, alkylenedioxy, aralkyloxy, CF₃ or —OCF₃; in combination with another antimycobacterial active agent.

23. The method of claim 22, wherein the capuramycin analogue has the formula I-A.

24. The method of claim 22, wherein the capuramycin analogue has the formula II.

25. The method of claim 22, wherein the capuramycin analogue has the structure: [chemical structure]
